(19) United States
(12) Patent Application Publication
(10) Pub. No.: US 2026/0078090 A1
(43) Pub. Date: Mar. 19, 2026

(54) N-SUBSTITUTED INDOLES AS 5-HYDROXYTRYPTAMINE (5-HT) SEROTONIN RECEPTOR AGONISTS AND ANTAGONISTS

(71) Applicant: Terran Biosciences Inc., Miami Beach, FL (US)

(72) Inventors: Samuel Clark, Miami Beach, FL (US); Matthew Duncton, Las Vegas, NV (US)

(21) Appl. No.: 19/402,111

(22) Filed: Nov. 26, 2025

Related U.S. Application Data
(63) Continuation of application No. 19/183,730, filed on Apr. 18, 2025, now abandoned.
Application No. 19/183,730 is a continuation of application No. 18/899,468, filed on Sep. 27, 2024, now abandoned.
Application No. 18/899,468 is a continuation of application No. 18/051,439, filed on Oct. 31, 2022, now abandoned.
(60) Provisional application No. 63/273,697, filed on Oct. 29, 2021.
(60) Provisional application No. 63/278,419, filed on Nov. 11, 2021.
(60) Provisional application No. 63/306,935, filed on Feb. 4, 2022.
(60) Provisional application No. 63/390,834, filed on Jul. 20, 2022.
(60) Provisional application No. 63/407,521, filed on Sep. 16, 2022.

(51) Int. Cl. C07D 209/32; C07D 209/30; C07D 471/04; C07D 487/04; C07D 491/048; C07D 491/052; C07D 495/04; C07D 513/04

(52) U.S. Cl. CPC C07D 209/32; C07D 209/30; C07D 471/04; C07D 487/04; C07D 491/048; C07D 491/052; C07D 495/04; C07D 513/04; C07B 2200/05

(57) ABSTRACT

Disclosed herein are compounds of the formula

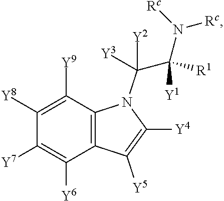

as well as methods for their use in treating neurologic and brain disorders.

### CROSS REFERENCE TO RELATED APPLICATIONS

The present application is a continuation of U.S. patent application Ser. No. 19/183,730, filed Apr. 18, 2025, which is a continuation of U.S. patent application Ser. No. 18/899,468, filed Sep. 27, 2024, which is a continuation of U.S. patent application Ser. No. 18/051,439, filed Oct. 31, 2022, now Abandoned, which claims priority to, and the benefit of, U.S. Provisional Application Nos. 63/273,697 filed Oct. 29, 2021, 63/278,419 filed Nov. 11, 2021, 63/306,935 filed Feb. 4, 2022, 63/390,834 filed Jul. 20, 2022, and 63/407,521 filed Sep. 16, 2022. The contents of the aforementioned patent applications are incorporated herein by reference in their entirety.

### FIELD

The present disclosure relates to N-substituted indole compounds and their use to treat brain and neurological disorders. The disclosure further relates to the provision of isotopically enriched compounds with improved characteristics.

### BACKGROUND

Major depressive disorder and related neuropsychiatric diseases are among the leading causes of disability worldwide. Despite recent advances, there remains a need for new therapeutics to support treatment of debilitating neuropsychiatric diseases.

Recently, psychedelic compounds have received renewed interest for the treatment of depression and other disorders. For example, the Food and Drug Administration (FDA) recently approved the dissociative anesthetic ketamine for treatment-resistant depression, making it the first mechanistically distinct medicine to be introduced to psychiatry in nearly thirty years. Ketamine is a member of a class of compounds known as psychoplastogens. Psychoplastogens promote neuronal growth through a mechanism involving the activation of AMPA receptors, the tropomyosin receptor kinase B (TrkB), and the mammalian target of rapamycin (mTOR). As pyramidal neurons in the PFC exhibit top-down control over areas of the brain controlling motivation, fear, and reward, these effects support clinical development of psychoplastogenic compounds for their antidepressant, anxiolytic, and anti-addictive effects properties.

A common pharmacophore in psychoactive compounds, particularly psychedelic compounds appears to be the N,N-dimethyltryptamine (DMT) skeleton. Recently, DMT was used as the starting point for identifying psychoplastogenic compounds (WO 2020/176597). However, known DMT derivatives, like many current medicines exhibit pharmacokinetic properties that undermine their use in clinical treatment. For example, such compounds may have undesirable absorption, distribution, metabolism and/or excretion (ADME) properties that prevent their wider use or limit their use in certain indications. While these compounds are useful in a variety of in vitro and in vivo contexts, there remains a need for compounds with improved effects and increased duration of actions. Compounds with such improved characteristics are disclosed herein.

### SUMMARY

The present disclosure relates to N-substituted indole compounds for the treatment of neurological and psychiatric disorders. In one embodiment the compounds have improved efficacy, improved pharmacokinetic properties or both. In one embodiment the disclosed compounds are isotopically enriched at one or more position.

In one aspect of the disclosed embodiments, the compounds are represented by Formula I

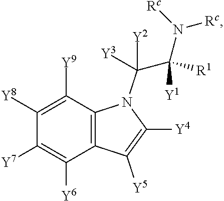

or
an enantiomer or diastereomer thereof wherein
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁷, Y⁸ and Y⁹ are each independently R^{b}, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{a}, —OR², —NO₂, —CN, —C(O)R^{b}, —C(O)OR^{b}, —OC(O)R^{b}, —OC(O)OR^{b}, —N(R^{yc}R^{yc}), —N(R^{b})C(O)R^{b}, —C(O)N(R^{yc}R^{yc}), —N(R^{b})C(O)OR^{b}, —OC(O)N(R^{c}R^{c}), —N(R^{b})C(O)N(R^{yc}R^{yc}), —C(O)C(O)N(R^{yc}R^{yc}), —SF₅, —S—R^{a}, —S—R^{b}, —S(O)R^{a}, —S(O)R^{b}, —S(O₂)R^{a}, —S(O₂)R^{b}, —S(O)₂N(R^{yc}R^{yc}), S(O)(N(R^{d})R^{b}, C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{a} is C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₆₋₁₀ aryl, C₅₋₁₀ heteroaryl, or C₄₋₁₆ alkyl-heteroaryl;
R^{b} is, for each occurrence, independently hydrogen or C₁₋₆ alkyl;
R^{d} is, for each occurrence, independently, R^{b} or C₃₋₈ cycloalkyl;
R^{e} is, for each occurrence, independently, —C(O)R^{b}, —C(O)OR^{b}, or —C(O)N(R^{c}R^{c});
R^{yc} is, for each occurrence, independently selected from hydrogen, C₁₋₆ alkyl, C₃₋₈ cycloalkyl, and C₄₋₁₄ alkyl-cycloalkyl, or two R^{yc} together with the nitrogen to which they are attached form a C₂₋₁₂ heterocycloalkyl; and
R^{c} is, for each occurrence, selected from hydrogen, C₁₋₆ alkyl, C₃₋₈ cycloalkyl, and C₄₋₁₄ alkyl-cycloalkyl, or two of R^{c} and R¹ together with the atoms to which they are attached to form a C₂₋₁₂ heterocycloalkyl;
alternatively, one of R^{c} and R¹ is combined with Y⁴ to form a C₅₋₁₂ heterocycloalkyl; alternatively, Y⁴ and Y⁵ are combined with the atoms to which they are each attached to form a C₄₋₈ cycloalkyl, C₄₋₁₀ heterocycloalkyl, or C₆₋₁₂ aryl; alternatively, Y⁶ and Y⁷, or Y⁷ and Y⁸ are combined with the atoms to which they are each attached to form a C₄₋₆ cycloalkyl, C₄₋₆ heterocycloalkyl, C₆₋₁₂ aryl, or C₄₋₁₀ heteroaryl;
wherein each cycloalkyl, heterocycloalkyl, aryl and heteroaryl is optionally substituted by one or more fluoro, R^{d} and R^{e}.

In one aspect of the disclosed embodiments, the compounds are represented by Formula IA:

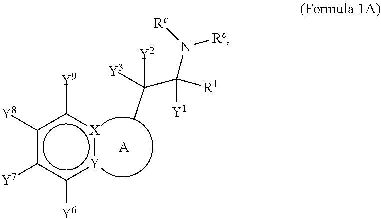

or
an enantiomer or diastereomer thereof, wherein:
Ring A is selected from:

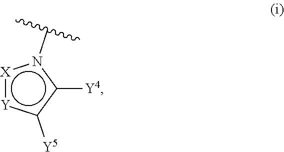

wherein X is C and Y is C;

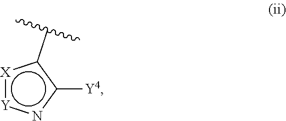

wherein X is N and Y is C;

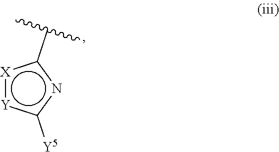

wherein X is N and Y is C;

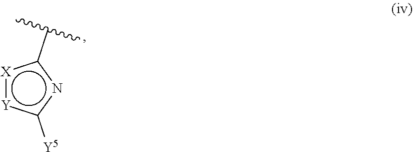

wherein X is C and Y is N;

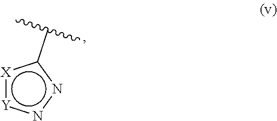

wherein X is N and Y is C; or

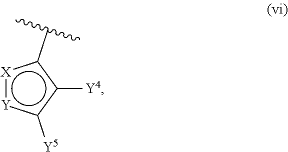

wherein X is N and Y is C;
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁷, Y⁸ and Y⁹ are each independently R^{b}, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{a}, —OR², —NO₂, —CN, —C(O)R^{b}, —C(O)OR^{b}, —OC(O)R^{b}, —OC(O)OR^{b}, —N(R^{yc}R^{yc}), —N(R^{b})C(O)R^{b}, —C(O)N(R^{yc}R^{yc}), —N(R^{b})C(O)OR^{b}, —OC(O)N(R^{c}R^{c}), —N(R^{b})C(O)N(R^{yc}R^{yc}), —C(O)C(O)N(R^{yc}R^{yc}), —SF₅, —S—R^{a}, —S—R^{b}, —S(O)R^{a}, —S(O)R^{b}, —S(O₂)R^{a}, —S(O₂)R^{b}, —S(O)₂N(R^{yc}R^{yc}), S(O)(N(R^{d})R^{b}, C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R² is selected from C₁₋₆ alkyl, C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl and C₄₋₁₆ alkyl-heteroaryl; or Y⁶ and R² are combined with the atoms to which they are each attached to form a C₄₋₆ heterocycloalkyl or C₄₋₁₀ heteroaryl;
R^{a} is C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₆₋₁₀ aryl, C₅₋₁₀ heteroaryl, or C₄₋₁₆ alkyl-heteroaryl;
R^{b} is, for each occurrence, independently hydrogen, deuterium, or C₁₋₆ alkyl;
R^{d} is, for each occurrence, independently, R^{b} or C₃₋₈ cycloalkyl;
R^{e} is, for each occurrence, independently, —C(O)R^{b}, —C(O)OR^{b}, or —C(O)N(R^{c}R^{c});
R^{yc} is, for each occurrence, independently selected from hydrogen, C₁₋₆ alkyl, C₃₋₈ cycloalkyl, and C₄₋₁₄ alkyl-cycloalkyl, or two R^{yc} together with the nitrogen to which they are attached form a C₂₋₁₂ heterocycloalkyl; and
R^{c} is, for each occurrence, selected from hydrogen, deuterium, C₁₋₆ alkyl, C₃₋₈ cycloalkyl, and C₄₋₁₄ alkyl-cycloalkyl, or two of R^{c} and R¹ together with the atoms to which they are attached to form a C₂₋₁₂ heterocycloalkyl;
alternatively, one of R^{c} and R¹ is combined with Y⁴ to form a C₅₋₁₂ heterocycloalkyl; alternatively, Y⁴ and Y⁵ are combined with the atoms to which they are each attached to form a C₄₋₈ cycloalkyl, C₄₋₁₀ heterocycloalkyl, or C₆₋₁₂ aryl;
alternatively, Y⁶ and Y⁷, or Y⁷ and Y⁸ are combined with the atoms to which they are each attached to form a C₄₋₆ cycloalkyl, C₄₋₆ heterocycloalkyl, C₆₋₁₂ aryl, or C₄₋₁₀ heteroaryl;
wherein each cycloalkyl, heterocycloalkyl, aryl and heteroaryl is optionally substituted by one or more fluoro, R^{d} and R^{e};
with the proviso that (1) when Y⁹, Y⁸, Y⁷, or Y⁶ is —OMe, methyl, or fluoro, and (2) Ring A is

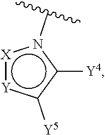

wherein X is C and Y is C, then at least one of Y⁹, Y⁸, Y⁷, Y⁶, Y⁸, Y⁴, Y³, Y², Y¹, R¹, or R^{c} is deuterium or is substituted with deuterium;
or a pharmaceutically acceptable salt thereof.

In some embodiments the compounds are represented by Formula IB

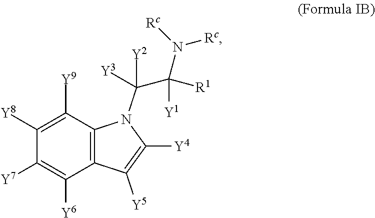

or
an enantiomer or diastereomer thereof wherein
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁷, Y⁸ and Y⁹ are each independently R^{b}, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{a}, —OR², —NO₂, —CN, —C(O)R^{b}, —C(O)OR^{b}, —OC(O)R^{b}, —OC(O)OR^{b}, —N(R^{yc}R^{yc}), —N(R^{b})C(O)R^{b}, —C(O)N(R^{yc}R^{yc}), —N(R^{b})C(O)OR^{b}, —OC(O)N(R^{c}R^{c}), —N(R^{b})C(O)N(R^{yc}R^{yc}), —C(O)C(O)N(R^{yc}R^{yc}), —SF₅, —S—R^{a}, —S—R^{b}, —S(O)R^{a}, —S(O)R^{b}, —S(O₂)R^{a}, —S(O₂)R^{b}, —S(O)₂N(R^{yc}R^{yc}), S(O)(N(R^{d})R^{b}, C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R² is selected from C₁₋₆ alkyl, C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl and C₄₋₁₆ alkyl-heteroaryl; or Y⁶ and R² are combined with the atoms to which they are each attached to form a C₄₋₆ heterocycloalkyl or C₄₋₁₀ heteroaryl;
R^{a} is C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₆₋₁₀ aryl, C₅₋₁₀ heteroaryl, or C₄₋₁₆ alkyl-heteroaryl;
R^{b} is, for each occurrence, independently hydrogen, deuterium, or C₁₋₆ alkyl;
R^{d} is, for each occurrence, independently, R^{b} or C₃₋₈ cycloalkyl;
R^{e} is, for each occurrence, independently, —C(O)R^{b}, —C(O)OR^{b}, or —C(O)N(R^{c}R^{c});
R^{yc} is, for each occurrence, independently selected from hydrogen, deuterium, C₁₋₆ alkyl, C₃₋₈ cycloalkyl, and C₄₋₁₄ alkyl-cycloalkyl, or two R^{yc} together with the nitrogen to which they are attached form a C₂₋₁₂ heterocycloalkyl; and
R^{c} is, for each occurrence, selected from hydrogen, deuterium, C₁₋₆ alkyl, C₃₋₈ cycloalkyl, and C₄₋₁₄ alkyl-cycloalkyl, or two of R^{c} and R¹ together with the atoms to which they are attached to form a C₂₋₁₂ heterocycloalkyl;
alternatively, one of R^{c} and R¹ is combined with Y⁴ to form a C₅₋₁₂ heterocycloalkyl;
alternatively, Y⁴ and Y⁵ are combined with the atoms to which they are each attached to form a C₄₋₈ cycloalkyl, C₄₋₁₀ heterocycloalkyl, or C₆₋₁₂ aryl; alternatively, Y⁶ and Y⁷, or Y⁷ and Y⁸ are combined with the atoms to which they are each attached to form a C₄₋₆ cycloalkyl, C₄₋₆ heterocycloalkyl, C₆₋₁₂ aryl, or C₄₋₁₀ heteroaryl;
   wherein each cycloalkyl, heterocycloalkyl, aryl and heteroaryl is optionally substituted by one or more fluoro, R^{d} and R^{e};
with the proviso that (1) when Y⁹, Y⁸, Y⁷, or Y⁶ is —OMe, methyl, or fluoro, then at least one of Y⁹, Y⁸, Y⁷, Y⁶, Y⁵, Y⁴, Y³, Y², Y¹, R¹, or R^{c} is deuterium or is substituted with deuterium.

In some embodiments, the compound is of Formula IV:

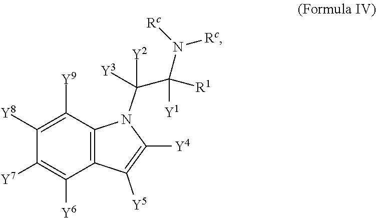

wherein Y¹ is hydrogen, deuterium, —CH₃, or —CD₃;
Y², Y³, Y⁴, Y⁵, Y⁸, and Y⁹ are each, independently, hydrogen or deuterium;
Y⁷ is:
   (i) —O—R², —S—R^{a}, —S(O)₂—R^{a}, —CN, -or S(F)₅;
      wherein R² is a C₃₋₈ cycloalkyl, CH₃, CD₃, or combines with Y⁶ to form a C₄₋₅ heterocycloalkyl; and
      R^{a} is a C₃₋₈ cycloalkyl or CH₃; or
   (ii) Y⁷ and Y⁶, together with the atoms to which they are attached, combine to form a C₆₋₁₀ aryl or a C₂₋₅ heteroaryl ring;
each R^{c} is, independently, CH₃ or CD₃;
R¹ is CH₃ or CD₃; and
Y⁶ is hydrogen, deuterium, or combines with R² to form a C₄₋₅ heterocycloalkyl or C₅₋₆ cycloalkyl;
   with the proviso that when R² is CH₃, then at least one of Y¹, Y², Y³, Y⁴, Y⁵, Y⁸, and Y⁹ are deuterium, or at least one R_{c} is CD₃, or R₁ is CD₃;
   or a pharmaceutically acceptable salt thereof.

In some embodiments, Y⁶ and Y⁷, together with the atoms to which they are attached, form a C₄₋₆ cycloalkyl, C₄₋₆ heterocycloalkyl, C₆₋₁₀ aryl, or C₄₋₁₀ heteroaryl. In some embodiments, Y⁷ is —O—R², —S—R^{a}, —S(O)₂—R^{a}, or —S(F)₅. In some embodiments, Y⁷ is —OCH₃, —OCD₃, —O-cyclopropyl, —S-cyclopropyl, or —S(O)₂-cyclopropyl.

In some embodiments, the compound is of Formula II′

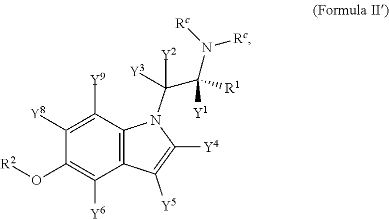

or
an enantiomer or diastereomer thereof, wherein
R² is selected from C₁₋₆ alkyl, C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl and C₄₋₁₆ alkyl-heteroaryl;
or a pharmaceutically acceptable salt thereof.

In some embodiments, R² is —CH₃, —CD₃, or cyclopropyl.

In some embodiments, the compound is of Formula Ix

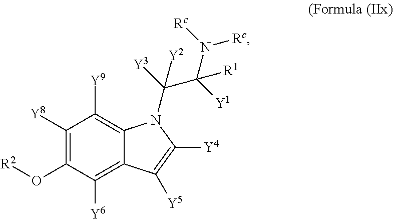

or
an enantiomer or diastereomer thereof wherein
R¹ is selected from C₁₋₆ alkyl, C₃₋₈ cycloalkyl, or C₄₋₁₄ alkyl-cycloalkyl;
Y¹, Y², Y², Y³, Y⁴, Y⁵, Y⁶, Y⁸ and Y⁹ are each independently selected from hydrogen, deuterium, halogen and C₁₋₆ alkyl,
R² is selected from haloalkyl and C₃₋₈ cycloalkyl, or R² and Y⁶ together form a C₄₋₁₀ heterocycloalkyl, or C₄₋₁₂ heteroaryl; and
R^{c} is, for each occurrence, selected from C₁₋₆ alkyl, C₃₋₈ cycloalkyl, or C₄₋₁₄ alkyl-cycloalkyl, or two of R^{c} and R¹ together with the atoms to which they are attached to form a C₂₋₁₂ heterocycloalkyl;
or a pharmaceutically acceptable salt thereof.

In some embodiments, the compound is of Formula XIV:

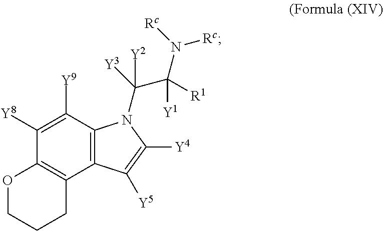

or the compound is of Formula XV:

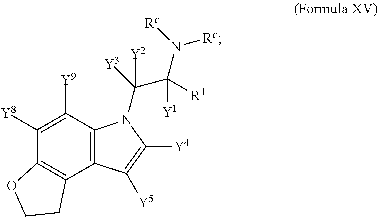

or a pharmaceutically acceptable salt thereof.

In some embodiments, each R^{c} is, independently, CH₃ or CD₃. In some embodiments, Y² and Y³ are each, independently, H or D. In some embodiments, R¹ is CH₃ or CD₃. In some embodiments, Y¹ is H, D, CH₃, or CD₃. In some embodiments, Y⁸, Y⁹, Y⁵, and Y⁴ are hydrogen.

In some embodiments, the compound is of Formula III′:

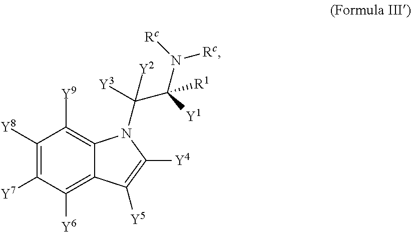

or
an enantiomer or diastereomer thereof wherein
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁸ and Y⁹ are each independently selected from hydrogen, deuterium, halogen and C₁₋₆ alkyl,
Y⁷ is selected from —S(F)₅ or —S—R²;
R² is selected from CH₃ or C₃₋₈ cycloalkyl, or R² and Y⁶ together form a C₄₋₁₀ heterocycloalkyl, or C₄₋₁₂ heteroaryl; and
R^{c} is, for each occurrence, selected from C₁₋₆ alkyl, C₃₋₈ cycloalkyl, or C₄₋₁₄ alkyl-cycloalkyl, or two of R^{c} and R¹ together with the atoms to which they are attached to form a C₂₋₁₂ heterocycloalkyl;
or a pharmaceutically acceptable salt thereof.

In some embodiments, R² is cyclopropyl.

In some embodiments, the compound is of Formula V:

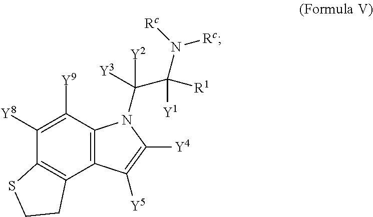

or wherein the compound is of Formula VI:

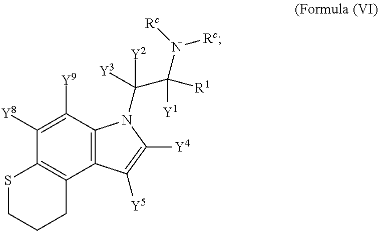

or a pharmaceutically acceptable salt thereof, wherein
each R^{c} is methyl;
Y¹ is H or methyl;
R¹ is methyl; and
   Y², Y³, Y⁴, Y⁵, Y⁸, and Y⁹ are hydrogen.

In some embodiments, the compound is of Formula VII:

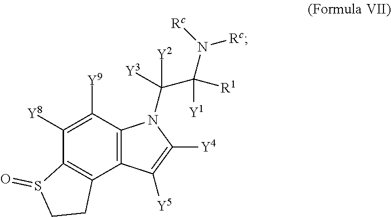

or the compound is of Formula VIII:

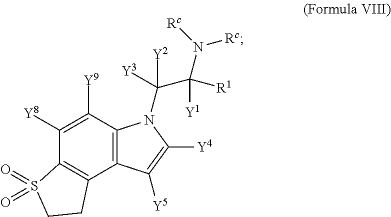

or the compound is of Formula IX:

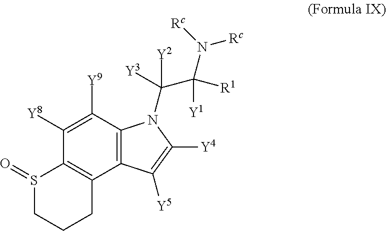

or the compound is of Formula X:

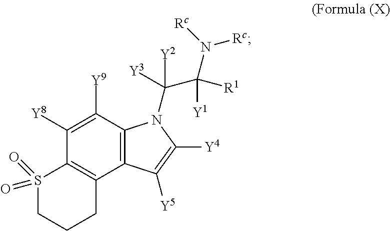

or a pharmaceutically acceptable salt thereof;
wherein each R^{c} is methyl;
   Y¹ is H or methyl;
   R¹ is methyl; and
      Y², Y³, Y⁴, Y⁵, Y⁸, and Y⁹ are hydrogen.

In some embodiments, the compound is of Formula XI:

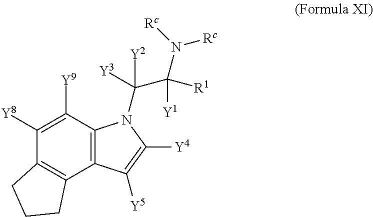

or the compound is of Formula XII:

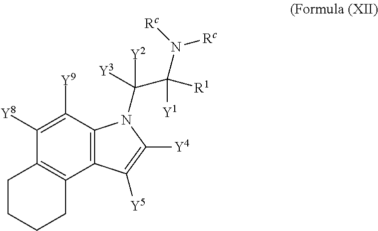

or the compound is of Formula XIII:

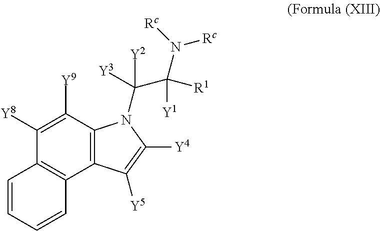

or a pharmaceutically acceptable salt thereof;
   wherein each R^{c} is methyl;
   Y¹ is H or methyl;
   R¹ is methyl; and
      Y², Y³, Y⁴, Y⁵, Y⁸, and Y⁹ are hydrogen.

In some embodiments of the proceeding compounds, at least one of Y¹, Y², Y³, Y⁴, Y⁵, Y⁶, Y⁷, Y⁸ and Y⁹ is deuterium. In some embodiments, at least one R^{c} is deuterium. In some embodiments, at least one of R¹, R² and R^{c} is deuterium.

In some embodiments, the compound is:

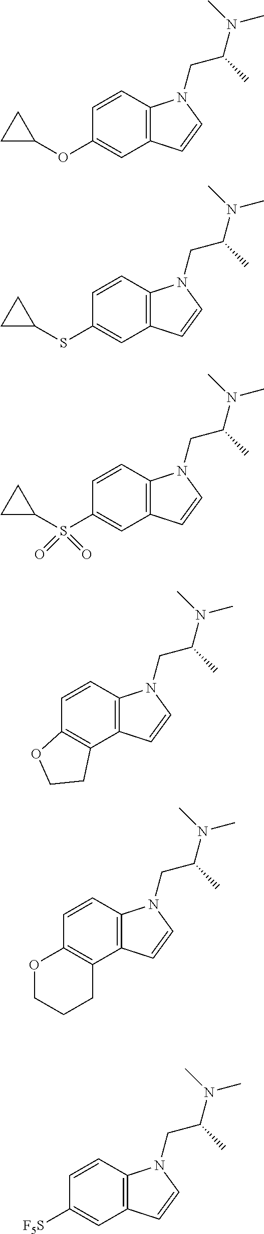

or an enantiomer or diastereomer thereof, or a pharmaceutically acceptable salt thereof.

In some embodiments, the compound is:

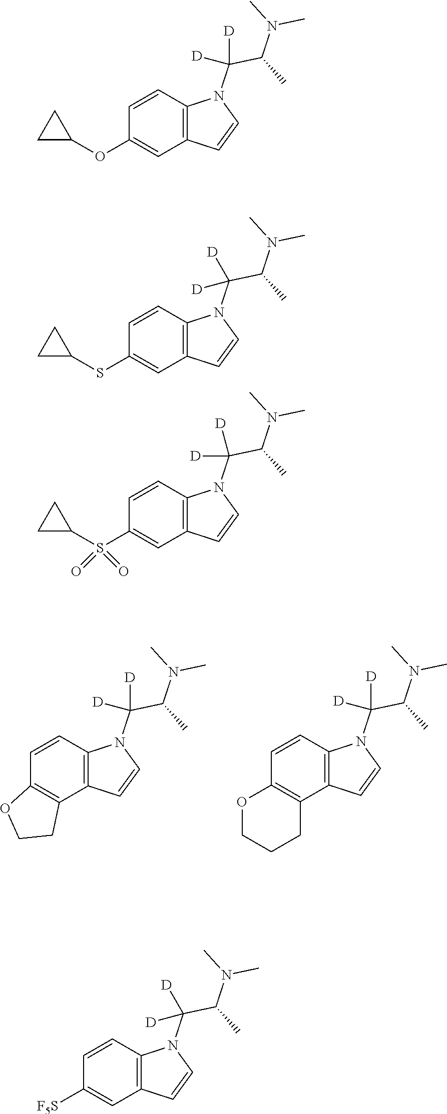

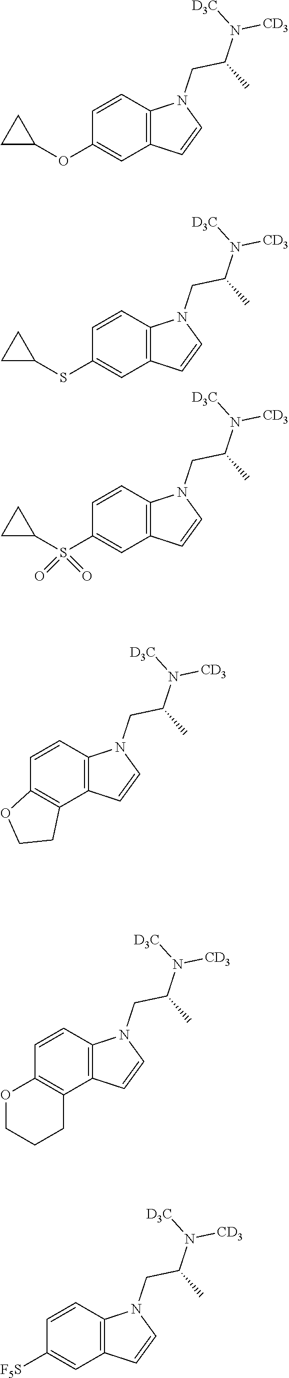

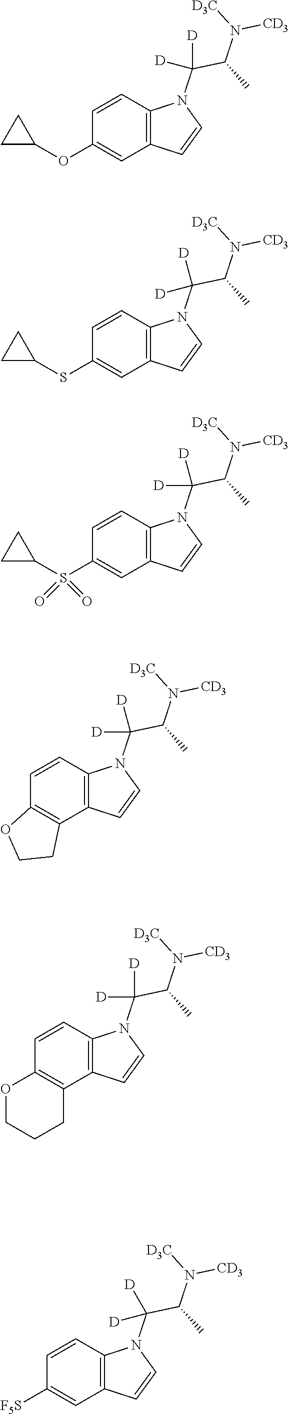

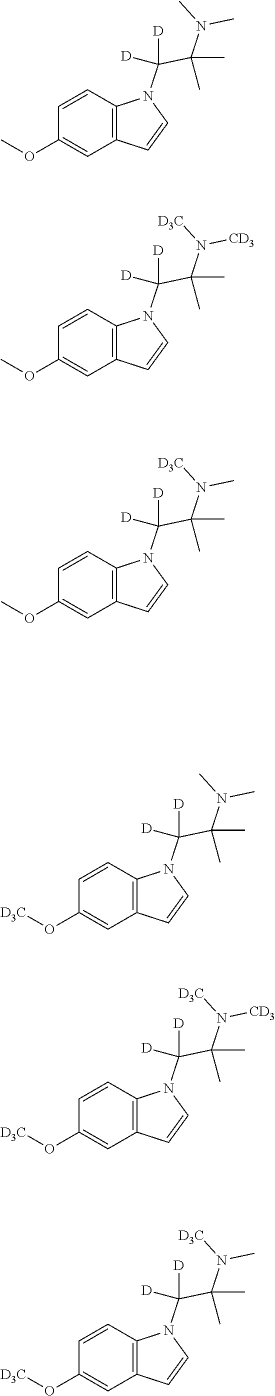

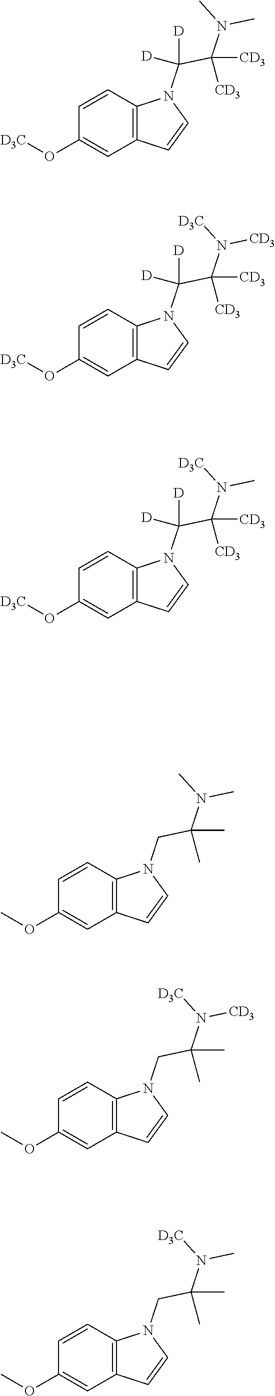

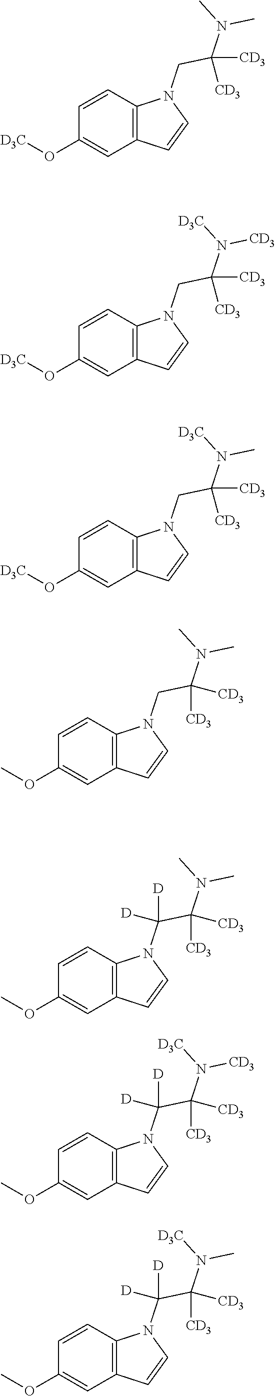

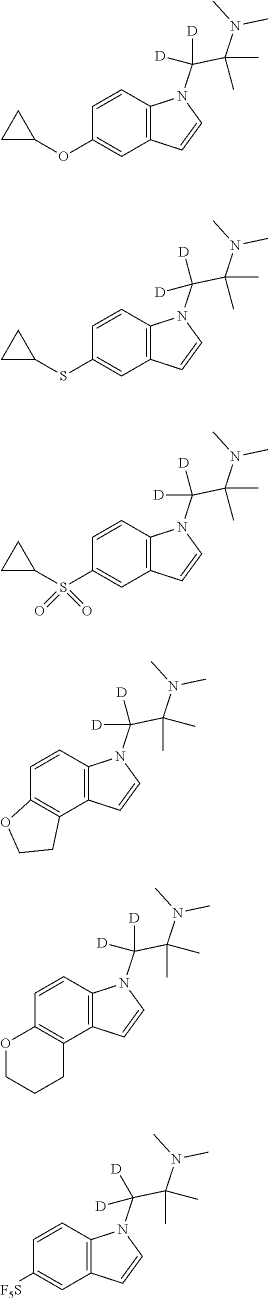

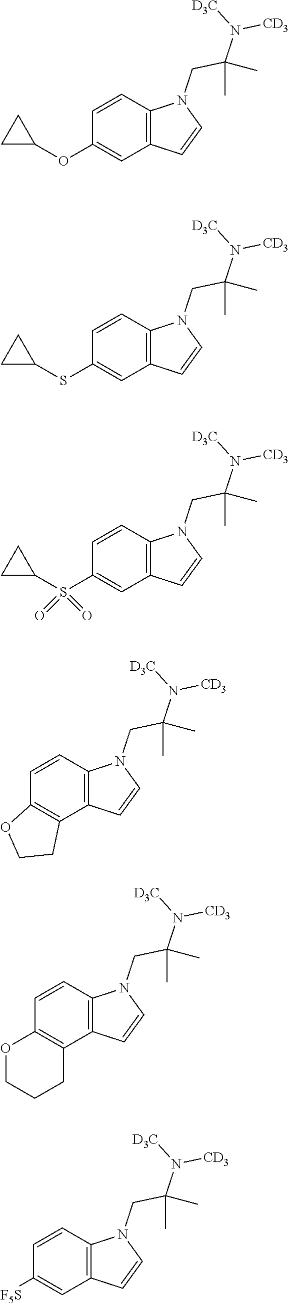

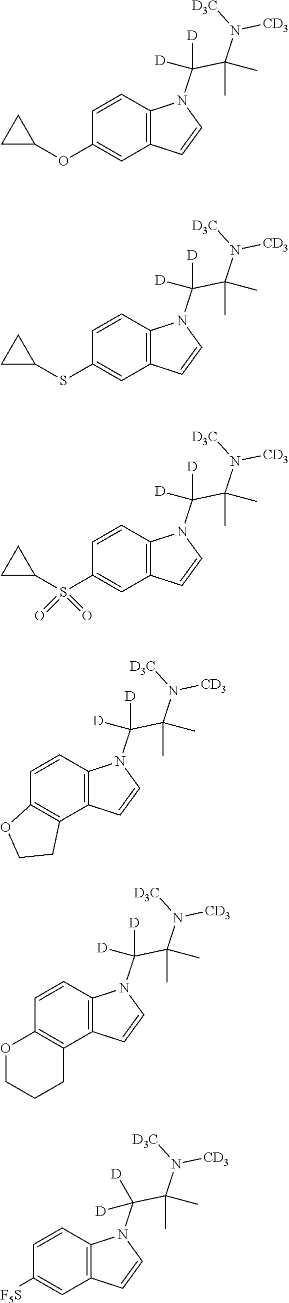

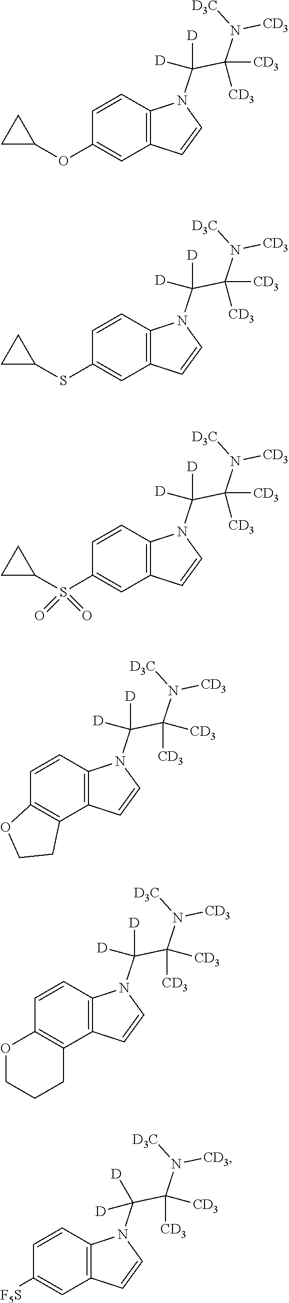

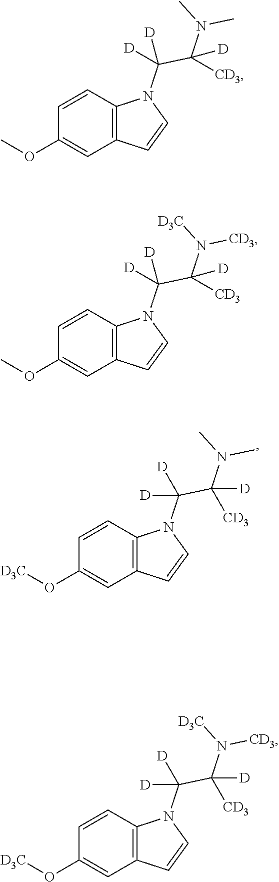

or an enantiomer or diastereomer thereof, or a pharmaceutically acceptable salt thereof.

In some embodiments, the compound is:

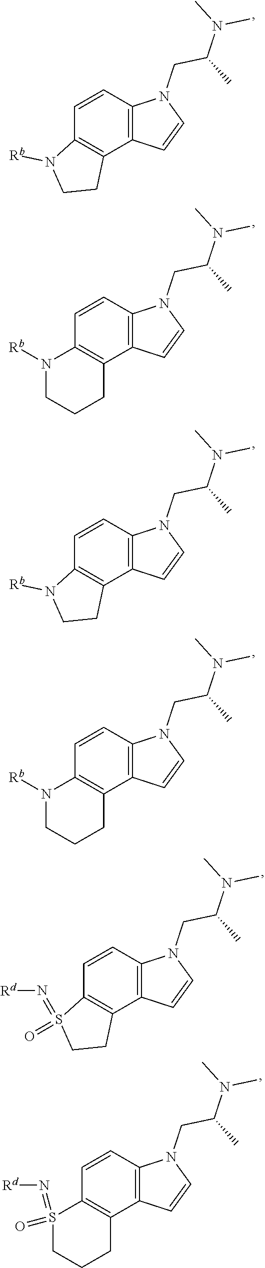

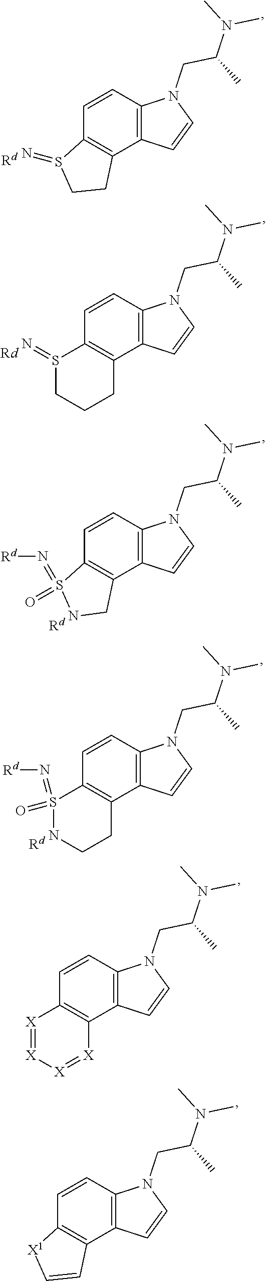

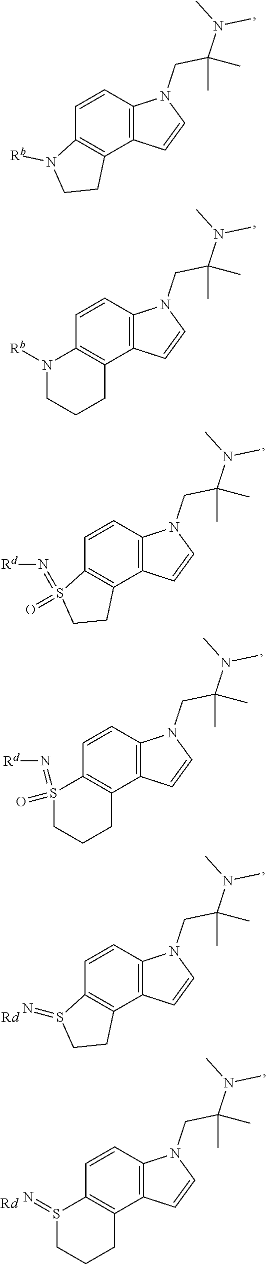

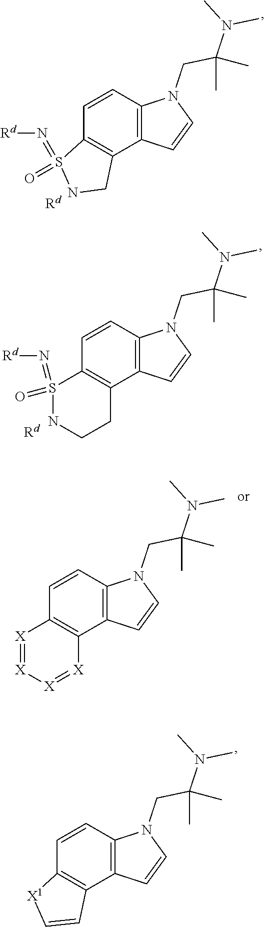

wherein
X is, independently for each occurrence, CH or N;
X¹ is selected from O, S, NR and NR^{e}; or
an enantiomer or diastereomer thereof, or a pharmaceutically acceptable salt thereof.

In some embodiments, the compound is:

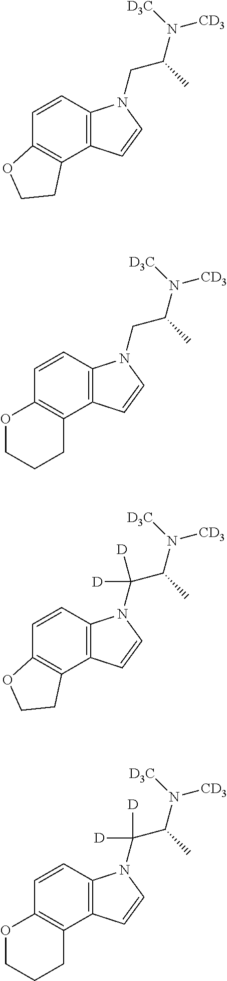 [SMILES: [2H]C([2H])([2H])N([C@H](C)Cn1ccc2c3c(ccc21)OCC3)C([2H])([2H])[2H] ; [2H]C([2H])([2H])N([C@H](C)Cn1ccc2c3c(ccc21)OCCC3)C([2H])([2H])[2H] ; [2H]C([2H])([2H])N([C@H](C)C([2H])([2H])n1ccc2c3c(ccc21)OCC3)C([2H])([2H])[2H] ; [2H]C([2H])([2H])N([C@H](C)C([2H])([2H])n1ccc2c3c(ccc21)OCCC3)C([2H])([2H])[2H]]

or an enantiomer or diastereomer thereof, or a pharmaceutically acceptable salt thereof.

In some embodiments, the compound is:

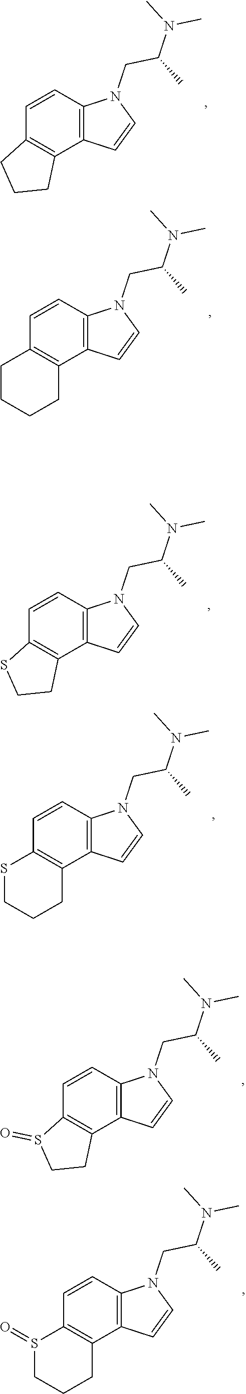 [SMILES: C[C@H](Cn1ccc2c3c(ccc21)CCC3)N(C)C ; C[C@H](Cn1ccc2c3c(ccc21)CCCC3)N(C)C ; C[C@H](Cn1ccc2c3c(ccc21)SCC3)N(C)C ; C[C@H](Cn1ccc2c3c(ccc21)SCCC3)N(C)C ; C[C@H](Cn1ccc2c3c(ccc21)S(=O)CC3)N(C)C ; C[C@H](Cn1ccc2c3c(ccc21)S(=O)CCC3)N(C)C]

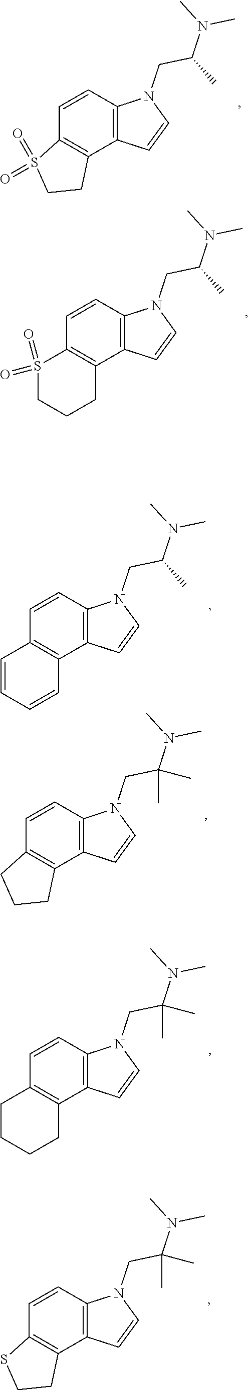 [SMILES: C[C@H](Cn1ccc2c3c(ccc21)S(=O)(=O)CC3)N(C)C ; C[C@H](Cn1ccc2c3c(ccc21)S(=O)(=O)CCC3)N(C)C ; C[C@H](Cn1ccc2c3ccccc3ccc21)N(C)C ; CN(C)C(C)(C)Cn1ccc2c3c(ccc21)CCC3 ; CN(C)C(C)(C)Cn1ccc2c3c(ccc21)CCCC3 ; CN(C)C(C)(C)Cn1ccc2c3c(ccc21)SCC3]

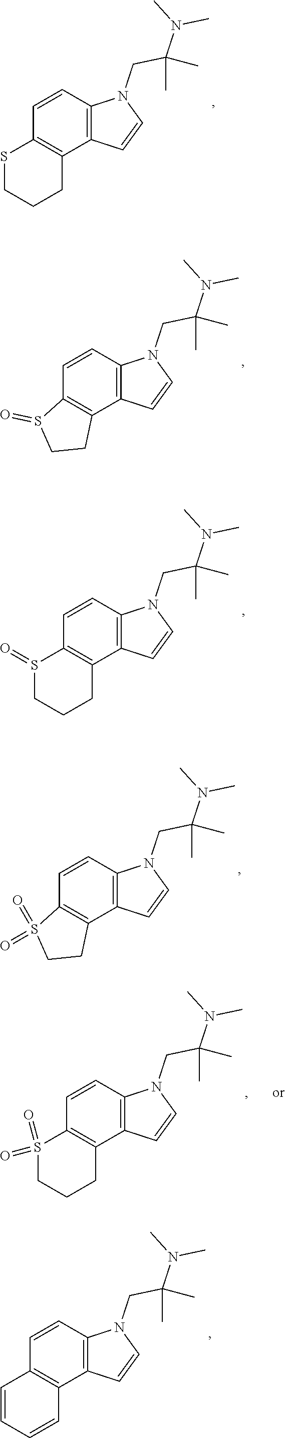 [SMILES: CN(C)C(C)(C)Cn1ccc2c3c(ccc21)SCCC3 ; CN(C)C(C)(C)Cn1ccc2c3c(ccc21)S(=O)CC3 ; CN(C)C(C)(C)Cn1ccc2c3c(ccc21)S(=O)CCC3 ; CN(C)C(C)(C)Cn1ccc2c3c(ccc21)S(=O)(=O)CC3 ; CN(C)C(C)(C)Cn1ccc2c3c(ccc21)S(=O)(=O)CCC3 ; CN(C)C(C)(C)Cn1ccc2c3ccccc3ccc21]

or a pharmaceutically acceptable salt thereof.

In some embodiments, the structure of any one of the compounds in Table 1.

In some embodiments, the present disclosure provides a pharmaceutical composition comprising a compound, or pharmaceutically acceptable salt thereof, of any one of claims 1-27.

In some embodiments, the present disclosure provides a method for increasing neuronal plasticity, comprising contacting a neuron with an effective amount of any compound or pharmaceutical composition disclosed herein. In some embodiments, contacting comprises administering the compound to a subject.

In some embodiments, the present disclosure provides a method for treating a neurological disorder or a psychiatric disorder, or both, comprising contacting a subject having the neurological disorder, psychiatric disorder or both with any compound or pharmaceutical composition disclosed herein. In some embodiments, the neurological disorder is a neurodegenerative disorder. In some embodiments, the neurological disorder or psychiatric disorder, or both, comprises depression, addiction, anxiety, or a post-traumatic stress disorder.

In some embodiments, the neurological disorder or psychiatric disorder, or both, comprises treatment resistant depression, suicidal ideation, major depressive disorder, bipolar disorder, schizophrenia, or substance use disorder. In some embodiments, the neurological disorder or psychiatric disorder, or both, comprises stroke, traumatic brain injury, or a combination thereof.

In some embodiments, the method further comprises administering to the subject an effective amount of an empathogenic agent. In some embodiments, the empathogenic agent is MDMA.

In some embodiments, the method further comprises administering a 5-HT_{2A} antagonist to the subject. In some embodiments, the 5-HT_{2A} antagonist is selected from MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, MDL-100,907, pimavanserin, nelotanserin and lorcaserin.

The foregoing and other objects, features, and advantages of the invention will become more apparent from the following detailed description.

### BRIEF DESCRIPTION OF THE FIGURES

FIG. 1. shows control dose response curves for the selected GPCR Biosensor Assays.

FIG. 2. illustrates the effect of AAZ, five representative compounds of the application, and 5-MeO-DMT on average cumulative head twitches in mice.

FIG. 3. provides a bar chart of average total head twitches induced after administration of AAZ, five representative compounds of the application, and 5-MeO-DMT.

### DETAILED DESCRIPTION

### I. General

Disclosed herein are N-substituted indoles, related N-containing heteroaryls, and isotopically labeled N-substituted indoles and related N-containing heteroaryls, or isotopologues. The presently disclosed isotopologues, e.g., the presently disclosed deuterated N-substituted indoles, are useful for the treatment of a variety of brain disorders and other conditions. Without limitation to any particular theory, it is believed that the present compounds increase neuronal plasticity, and increase at least one of translation, transcription, or secretion of neurotrophic factors. Moreover, the presently disclosed compounds have improved pharmacokinetic and pharmacodynamic properties as compared to previously disclosed molecules. In some embodiments, the improved pharmacokinetic and pharmacodynamic properties are due to the isotopic enrichment. In certain embodiments the isotopic labels of the present compounds allow monitoring of its pharmacodynamic and ADME behavior following in vivo administration. In some embodiments, the isotopically enriched compounds described herein provide better therapeutic potential for neurological diseases than known compounds.

### Terms and Abbreviations:

The term “isotopic enrichment factor” as used herein means the ratio between the isotopic abundance and the natural abundance of a specified isotope. It will be recognized that some variation of natural isotopic abundance occurs in a synthesized compound depending upon the origin of chemical materials used in the synthesis. Thus, a preparation of any compound will inherently contain small amounts of isotopologues, including deuterated isotopologues. The concentration of naturally abundant stable hydrogen isotopes, notwithstanding this variation, is small and immaterial as compared to the degree of stable isotopic substitution of compounds of this disclosure. In a compound of this disclosure, when a particular position is designated as having a particular isotope, such as deuterium, it is understood that the abundance of deuterium at that position is substantially greater than the natural abundance of deuterium, which is about 0.015% (on a mol/mol basis). A position designated as a particular isotope will have a minimum isotopic enrichment factor of at least 3000 (45% incorporation of the indicated isotope). Thus, isotopically enriched compounds disclosed herein having deuterium will have a minimum isotopic enrichment factor of at least 3000 (45% deuterium incorporation) at each atom designated as deuterium in the compound. Such compounds may be referred to herein as “deuterated” compounds.

In other embodiments, disclosed compounds have an isotopic enrichment factor for each designated atom of at least 3500 (52.5%). For example, for such disclosed compounds that are deuterium isotopologues, the compounds have an isotopic enrichment factor for each designated hydrogen atom of at least 3500 (520.5% deuterium incorporation at each designated atom), at least 4000 (60% deuterium incorporation), at least 4500 (670.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (990.5% deuterium incorporation). Compounds with a deuterium enrichment factor of at least 3500 are referred to herein as “deuterated” compounds.

In the compounds of this disclosure any atom not specifically designated as a particular isotope is meant to represent any stable isotope of that atom. Unless otherwise stated, when a position is designated specifically as “H” or “protium”, the position is understood to have hydrogen at about its natural abundance isotopic composition.

The term “isotopologue” refers to a species that has the same chemical structure and formula as another compound, with the exception of the isotopic composition at one or more positions, e.g., H vs. D. Thus, isotopologues differ in their isotopic composition.

Abbreviations used: DMT, A,A-dimethyltryptamine; PFC, prefrontal cortex; 5-HT2A, serotonin 2A; MPO, multiparameter optimization; LSD, lysergic acid diethylamide: TPSA, total polar surface area; MAP2, microtubule-associated protein 2; Nₘₐₓ, maximum number of crossings; 5-HT2B, serotonin 2B; DJV, days in vitro; VEH, vehicle; KET, ketamine; SEM, standard error of the mean; ANOVA, analysis of variance; DOM, 2,5-dimethoxy-4-methylamphetamine; OMe, methoxy; OBn, benzyloxy; F, fluoro; μM, micromolar; nM, nanomolar; pM, picomolar; V, vehicle; K, ketamine; ATR, attenuated total reflectance; FT-IR, Fourier transform infrared spectroscopy; UHPLC, ultra-high performance liquid chromatography; LRMS, low-resolution mass spectrometry; BSA, bovine serum albumin; DPBS, Dulbecco's phosphate-buffered saline; mTOR, mammalian target of rapamycin; AMP A, α-amino-3-hydroxy-5-methyl-4-isoxazolepropionic acid; TrkB, tropomyosin receptor kinase B; HTR, head-twitch response.

“Alkyl” refers to a straight saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, and the like. Alkyl can also refer to alkyl groups having up to 20 carbons atoms, such as, but not limited to heptyl, octyl, nonyl, decyl and the like. Alkyl groups can be substituted or unsubstituted.

“Branched alkyl” refers to a branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, and the like. Alkyl can also refer to alkyl groups having up to 20 carbons atom, such as, but not limited to heptyl, octyl, nonyl, decyl and the like. Alkyl groups can be substituted or unsubstituted.

“Alkylene” refers to a straight, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)n— where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted. “Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl and the like.

Alkynyl groups can be substituted or unsubstituted.

“Cycloalkyl” refers to a saturated or partially unsaturated, monocyclic, bicyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Bicyclic compounds include spirocyclic compounds, fused bicyclic compounds and bridged bicyclic compounds. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. The alkylene component can include any number of carbons, such as C₁₋₆, C₁₋₂, C₁₋₃, C₁₋₄, C₁₋₅, C₃₋₆, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycloalkyl” refers to a cycloalkyl as defined above, having from 3 to 12 ring members wherein at least one carbon is replaced by a heteroatom selected from N, O and S. Heterocycloalkyl groups contain between 1 and 4 heteroatoms, unless otherwise specified. Heterocycloalkyl includes bicyclic compounds which include a heteroatom. The term “bicyclic” includes spirocyclic compounds, fused bicyclic compounds, and bridged bicyclic compounds. The heteroatoms can also be oxidized, such as, but not limited to, —S(O)— and —S(O)₂—. Unless otherwise specified, heterocycloalkyl groups can include 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

“Alkyl-heterocycloalkyl” refers to a radical having an alkylene component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. Unless otherwise specified, the alkyl component can include any number of carbons, such as C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. The heterocycloalkyl component is as defined above. Alkyl-heterocycloalkyl groups can be substituted or unsubstituted.

“Halogen” or “halo” refers to fluorine, chlorine, bromine and iodine or the corresponding fluoro, chloro, bromo and iodo radicals.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, and the like. In some instances, the term “perfluoro” or “perhalo” can be used to define a compound or radical where all the hydrogens are replaced with fluorine or another halogen. For example, perfluoromethyl refers to 1,1,1-trifluoromethyl or —CF₃.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy and the like. The alkoxy groups can be further substituted with a variety of substituents described within. Alkoxy groups can be substituted or unsubstituted.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, and the like.

“Amine” refers to an —N(R)₂ group where the R groups can be hydrogen, deuterium, alkyl, alkenyl, alkynyl, cycloalkyl, heterocycloalkyl, aryl, or heteroaryl, among others. The R groups can be the same or different. The amino groups can be primary (each R is hydrogen), secondary (one R is hydrogen) or tertiary (each R is other than hydrogen).

“Alkyl amine” refers to an alkyl group as defined within, having one or more amino groups. The amino groups can be primary, secondary or tertiary. The alkyl amine can be further substituted with a hydroxy group to form an amino-hydroxy group. Alkyl amines useful in the present invention include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present invention.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₁₋₄, C₃₋₅, C₃₋₆, C₄₋₅, C₄₋₆ and C₅₋₆. The aryl component is as defined above. Examples of alkyl-aryl groups include, but are not limited to, benzyl and ethyl-benzene. Alkyl-aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Heteroaryl groups can include any number of ring atoms, such as, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₄, C₂₋₃, C₂₋₄, C₂₋₅, C₂₋₆, C₃₋₄, C₃₋₅, C₃₋₆, C₄₋₅, C₄₋₆ and C₅₋₆. The heteroaryl component is as defined within. Alkyl-heteroaryl groups can be substituted or unsubstituted.

“Salt” refers to acid or base salts of the disclosed herein, e.g., pharmaceutically acceptable salts. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid, tartaric acid and the like) salts, quaternary ammonium (methyl iodide, ethyl iodide, and the like) salts. It is understood that the pharmaceutically acceptable salts are non-toxic. Additional suitable pharmaceutically acceptable salts are known to those of skill in the art. See, e.g., Remington: The Science and Practice of Pharmacy, volume I and volume II. (22^{nd} Ed., University of the Sciences, Philadelphia)., which is incorporated herein by reference.

The neutral forms of the compounds may be regenerated by contacting the salt with a base or acid and isolating the parent compound. The parent form of the compound differs from the various salt forms in certain physical properties, such as solubility in polar solvents, but otherwise the salts are equivalent to the parent form of the compound for the purposes of the present invention.

“Pharmaceutically acceptable salt” refers to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc. In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt. [0084] Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3. It is to be understood that all references to pharmaceutically acceptable salts include solvent addition forms (solvates) or crystal forms (polymorphs) as defined herein, of the same salt. It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc. As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention.

“Composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation.

“Isomers” refers to compounds with same chemical formula but different connectivity between the atoms in the molecule, leading to distinct chemical structures. Isomers include structural isomers and stereoisomers. Examples of structural isomers include, but are not limited to, tautomers, and regioisomers. Examples of stereoisomers include but are not limited to diastereomers and enantiomers.

“Administering” refers to any suitable mode of administration, including, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Subject” refers to an animal, such as a mammal, including, but not limited to, primates (e.g., humans), cows, sheep, goats, horses, dogs, cats, rabbits, rats, mice and the like. In certain embodiments, the subject is a human subject.

“Therapeutically effective amount” or “therapeutically sufficient amount” or “effective or sufficient amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses.

“Brain disorder” refers to a neurological disorder which affects the brain's structure and function. Brain disorders can include, but are not limited to, Alzheimer's, Parkinson's disease, psychological disorder, depression, treatment resistant depression, addiction, anxiety, post-traumatic stress disorder, suicidal ideation, major depressive disorder, bipolar disorder, schizophrenia, stroke, traumatic brain injury, and substance use disorder.

“Combination therapy” refers to a method of treating a disease or disorder, wherein two or more different pharmaceutical agents are administered in overlapping regimens so that the subject is simultaneously exposed to both agents. For example, the compounds of the invention can be used in combination with other pharmaceutically active compounds. The compounds of the invention can be administered simultaneously (as a single preparation or separate preparation) or sequentially to the other drug therapy. In general, a combination therapy envisions administration of two or more drugs during a single cycle or course of therapy.

“Neurotrophic factors” refers to a family of soluble peptides or proteins which support the survival, growth, and differentiation of developing and mature neurons.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT_{2A}) are modulators of the receptor.

“Agonism” refers to the activation of a receptor or enzyme by a modulator, or agonist, to produce a biological response.

“Agonist” refers to a modulator that binds to a receptor or enzyme and activates the receptor to produce a biological response. By way of example only, “5HT_{2A} agonist” can be used to refer to a compound that exhibits an EC₅₀ with respect to 5HT_{2A} activity of no more than about 100 mM. In some embodiments, the term “agonist” includes full agonists or partial agonists. “Full agonist” refers to a modulator that binds to and activates a receptor with the maximum response that any agonist can elicit at the receptor. “Partial agonist” refers to a modulator that binds to and activates a given receptor, but has partial efficacy, that is, less than the maximal response, at the receptor relative to a full agonist.

“Positive allosteric modulator” refers to a modulator that binds to a site distinct from the orthosteric binding site and enhances or amplifies the effect of an agonist.

“Antagonism” refers to the inactivation of a receptor or enzyme by a modulator, or antagonist. Antagonism of a receptor, for example, is when a molecule binds to the receptor and does not allow activity to occur.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

### Compounds:

Disclosed herein are N-substituted indoles and related compounds useful for the treatment of a variety of brain disorders and other conditions. In some embodiments, the N-substituted indoles and other heterocyclic compounds provided herein are 5-HT_{2A} modulators and promote neural plasticity (e.g., cortical structural plasticity). In one embodiment, the present compounds adopt a pharmacophore found in psychoactive compounds, particularly psychedelic compounds, namely the N,N-dimethyltryptamine (DMT) skeleton. However, previously-disclosed DMT analogs, like many medicines, exhibit undesirable pharmacokinetic properties that undermine their use in clinical treatment.

The present inventors observed that the metabolic properties of previously disclosed N-substituted indoles could be improved by isotopic enrichment, in particular, deuterium or tritium enrichment. In this approach, one attempts to slow the cytochrome p450 (CYP) mediated metabolism of a drug or to reduce the formation of undesirable metabolites by replacing one or more protium (1H) atoms with deuterium atoms. Deuterium is a safe, stable, non-radioactive isotope of hydrogen. Compared to protium, deuterium forms stronger bonds with carbon. In select cases, the increased bond strength imparted by deuterium can positively affect the pharmacokinetic properties of a drug, creating the potential for improved drug efficacy, safety, and/or tolerability. At the same time, because the size and shape of deuterium are essentially identical to those of protium, replacement of protium by deuterium would not be expected to affect the biochemical potency and selectivity of the drug as compared to the original chemical entity that contains only hydrogen. Tritium, ³H, forms still stronger bonds with carbon than deuterium. Thus, replacement of protium with tritium also can affect the pharmacokinetic properties of a molecule. Moreover, tritium is a beta emitter, meaning that enriching a molecule with tritium allows determination of pharmacokinetic and pharmacodynamic properties of the molecule to better understand its activity and ADME properties.

Accordingly, in certain embodiments, the present invention provides a compound of Formula I

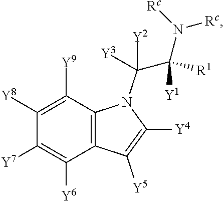

or
an enantiomer or diastereomer thereof wherein
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁷, Y⁸ and Y⁹ are each independently R^{b}, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{a}, —OR², —NO₂, —CN, —C(O)R^{b}, —C(O)OR^{b}, —OC(O)R^{b}, —OC(O)OR^{b}, —N(R^{yc}R^{yc}), —N(R^{b})C(O)R^{b}, —C(O)N(R^{yc}R^{yc}), —N(R^{b})C(O)OR^{b}, —OC(O)N(R^{c}R^{c}), —N(R^{b})C(O)N(R^{yc}R^{yc}), —C(O)C(O)N(R^{yc}R^{yc}), —SF⁵, —S—R^{a}, —S—R^{b}, —S(O)R^{a}, —S(O)R^{b}, —S(O₂)R^{a}, —S(O₂)R^{b}, —S(O)₂N(R^{yc}R^{yc}), S(O)(N(R^{d})R^{b}, C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R² is selected from C₁₋₆ alkyl, C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl and C₄₋₁₆ alkyl-heteroaryl; or Y⁶ and R² are combined with the atoms to which they are each attached to form a C₄₋₆ heterocycloalkyl or C₄₋₁₀ heteroaryl;
R^{a} is C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₆₋₁₀ aryl, C₅₋₁₀ heteroaryl, or C₄₋₁₆ alkyl-heteroaryl;
R^{b} is, for each occurrence, independently deuterium, hydrogen or C₁₋₆ alkyl;
R^{d} is, for each occurrence, independently, R^{b} or C₃₋₈ cycloalkyl;
R^{e} is, for each occurrence, independently, oxo, —N═R^{d}; —C(O)R^{b}, —C(O)OR^{b}, or —C(O)N(R^{c}R^{c});
R^{yc} is, for each occurrence, independently selected from hydrogen, deuterium, C₁₋₆ alkyl, C₃₋₈ cycloalkyl, and C₄₋₁₄ alkyl-cycloalkyl, or two R^{yc} together with the nitrogen to which they are attached form a C₂₋₁₂ heterocycloalkyl; and
R^{c} is, for each occurrence, selected from hydrogen, deuterium, C₁₋₆ alkyl, C₃₋₈ cycloalkyl, and C₄₋₁₄ alkyl-cycloalkyl, or two of R^{c} and R¹ together with the atoms to which they are attached to form a C₂₋₁₂ heterocycloalkyl;
alternatively, one of R^{c} and R¹ is combined with Y⁴ to form a C₅₋₁₂ heterocycloalkyl;
alternatively, Y⁴ and Y⁵ are combined with the atoms to which they are each attached to form a C₄₋₈ cycloalkyl, C₄₋₁₀ heterocycloalkyl, or C₆₋₁₂ aryl; alternatively, Y⁶ and Y⁷, or Y⁷ and Y⁸ are combined with the atoms to which they are each attached to form a C₄₋₆ cycloalkyl, C₄₋₆ heterocycloalkyl, C₆₋₁₂ aryl, or C₄₋₁₀ heteroaryl;
wherein each cycloalkyl, heterocycloalkyl, aryl and heteroaryl is optionally substituted by one or more fluoro, R^{d} and R^{e}.

In certain embodiments Y⁶ and Y⁷, together with the atoms to which they are attached, form a C₄₋₆ cycloalkyl, C₄₋₆ heterocycloalkyl, C₆₋₁₂ aryl, or C₄₋₁₀ heteroaryl.

In one aspect of the disclosed embodiments, the compounds are represented by Formula IA:

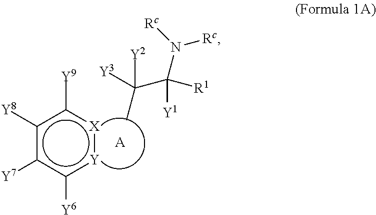

or
an enantiomer or diastereomer thereof, wherein:
Ring A is selected from:

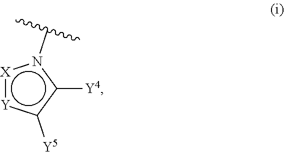

wherein X is C and Y is C;

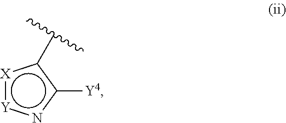

wherein X is N and Y is C;

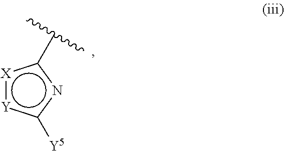

wherein X is N and Y is C;

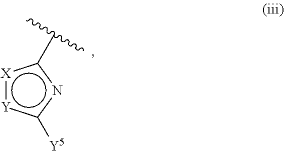

wherein X is C and Y is N;

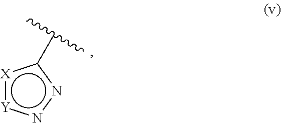

wherein X is N and Y is C; or

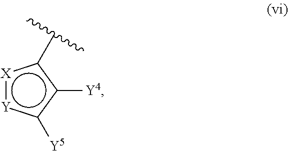

wherein X is N and Y is C;
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁷, Y⁸ and Y⁹ are each independently R^{b}, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{a}, —OR², —NO₂, —CN, —C(O)R^{b}, —C(O)OR^{b}, —OC(O)R^{b}, —OC(O)OR^{b}, —N(R^{yc}R^{yc}), —N(R^{b})C(O)R^{b}, —C(O)N(R^{yc}R^{yc}), —N(R^{b})C(O)OR^{b}, —OC(O)N(R^{c}R^{c}), —N(R^{b})C(O)N(R^{yc}R^{yc}), —C(O)C(O)N(R^{yc}R^{yc}), —SF₅, —S—R^{a}, —S—R^{b}, —S(O)R^{a}, —S(O)R^{b}, —S(O₂)R^{a}, —S(O₂)R^{b}, — S(O)₂N(R^{yc}R^{yc}), S(O)(N(R^{d})R^{b}, C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R² is selected from C₁₋₆ alkyl, C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl and C₄₋₁₆ alkyl-heteroaryl; or Y⁶ and R² are combined with the atoms to which they are each attached to form a C₄₋₆ heterocycloalkyl or C₄₋₁₀ heteroaryl;
R^{a} is C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₆₋₁₀ aryl, C₅₋₁₀ heteroaryl, or C₄₋₁₆ alkyl-heteroaryl;
R^{b} is, for each occurrence, independently hydrogen, deuterium, or C₁₋₆ alkyl;
R^{d} is, for each occurrence, independently, R^{b} or C₃₋₈ cycloalkyl;
R^{e} is, for each occurrence, independently, —C(O)R^{b}, —C(O)OR^{b}, or —C(O)N(R^{c}R^{c});
R^{yc} is, for each occurrence, independently selected from hydrogen, C₁₋₆ alkyl, C₃₋₈ cycloalkyl, and C₄₋₁₄ alkyl-cycloalkyl, or two R^{yc} together with the nitrogen to which they are attached form a C₂₋₁₂ heterocycloalkyl; and
R^{c} is, for each occurrence, selected from hydrogen, deuterium, C₁₋₆ alkyl, C₃₋₈ cycloalkyl, and C₄₋₁₄ alkyl-cycloalkyl, or two of R^{c} and R¹ together with the atoms to which they are attached to form a C₂₋₁₂ heterocycloalkyl;
alternatively, one of R^{c} and R¹ is combined with Y⁴ to form a C₅₋₁₂ heterocycloalkyl;
alternatively, Y⁴ and Y⁵ are combined with the atoms to which they are each attached to form a C₄₋₈ cycloalkyl, C₄₋₁₀ heterocycloalkyl, or C₆₋₁₂ aryl;
alternatively, Y⁶ and Y⁷, or Y⁷ and Y⁸ are combined with the atoms to which they are each attached to form a C₄₋₆ cycloalkyl, C₄₋₆ heterocycloalkyl, C₆₋₁₂ aryl, or C₄₋₁₀ heteroaryl;
wherein each cycloalkyl, heterocycloalkyl, aryl and heteroaryl is optionally substituted by one or more fluoro, R^{d} and R^{e};
with the proviso that (1) when Y⁹, Y⁸, Y⁷, or Y⁶ is —OMe, methyl, or fluoro, and (2) Ring A is

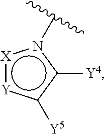

wherein X is C and Y is C, then at least one of Y⁹, Y⁸, Y⁷, Y⁶, Y⁵, Y⁴, Y³, Y², Y¹, R¹, or R^{c} is deuterium or is substituted with deuterium;
or a pharmaceutically acceptable salt thereof.

In some embodiments the compounds are represented by Formula IB

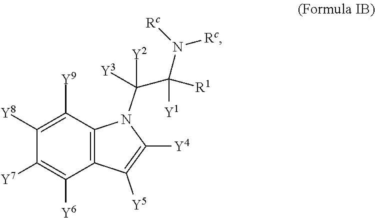

or
an enantiomer or diastereomer thereof wherein
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁷, Y⁸ and Y⁹ are each independently R^{b}, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{a}, —OR², —NO₂, —CN, —C(O)R^{b}, —C(O)OR^{b}, —OC(O)R^{b}, —OC(O)OR^{b}, —N(R^{yc}R^{yc}), —N(R^{b})C(O)R^{b}, —C(O)N(R^{yc}R^{yc}), —N(R^{b})C(O)OR^{b}, —OC(O)N(R^{c}R^{c}), —N(R^{b})C(O)N(R^{yc}R^{yc}), —C(O)C(O)N(R^{yc}R^{yc}), —SF₅, —S—R^{a}, —S—R^{b}, —S(O)R^{a}, —S(O)R^{b}, —S(O₂)R^{a}, —S(O₂)R^{b}, — S(O)₂N(R^{yc}R^{yc}), S(O)(N(R^{d})R^{b}, C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R² is selected from C₁₋₆ alkyl, C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl and C₄₋₁₆ alkyl-heteroaryl; or Y⁶ and R² are combined with the atoms to which they are each attached to form a C₄₋₆ heterocycloalkyl or C₄₋₁₀ heteroaryl;
R^{a} is C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₆₋₁₀ aryl, C₅₋₁₀ heteroaryl, or C₄₋₁₆ alkyl-heteroaryl;
R^{b} is, for each occurrence, independently hydrogen, deuterium, or C₁₋₆ alkyl;
R^{d} is, for each occurrence, independently, R^{b} or C₃₋₈ cycloalkyl;
R^{e} is, for each occurrence, independently, —C(O)R^{b}, —C(O)OR^{b}, or —C(O)N(R^{c}R^{c});
R^{yc} is, for each occurrence, independently selected from hydrogen, deuterium, C₁₋₆ alkyl, C₃₋₈ cycloalkyl, and C₄₋₁₄ alkyl-cycloalkyl, or two R^{yc} together with the nitrogen to which they are attached form a C₂₋₁₂ heterocycloalkyl; and
R^{c} is, for each occurrence, selected from hydrogen, deuterium, C₁₋₆ alkyl, C₃₋₈ cycloalkyl, and C₄₋₁₄ alkyl-cycloalkyl, or two of R^{c} and R¹ together with the atoms to which they are attached to form a C₂₋₁₂ heterocycloalkyl;
alternatively, one of R^{c} and R¹ is combined with Y⁴ to form a C₅₋₁₂ heterocycloalkyl;
alternatively, Y⁴ and Y⁵ are combined with the atoms to which they are each attached to form a C₄₋₈ cycloalkyl, C₄₋₁₀ heterocycloalkyl, or C₆₋₁₂ aryl; alternatively, Y⁶ and Y⁷, or Y⁷ and Y⁸ are combined with the atoms to which they are each attached to form a C₄₋₆ cycloalkyl, C₄₋₆ heterocycloalkyl, C₆₋₁₂ aryl, or C₄₋₁₀ heteroaryl;
   wherein each cycloalkyl, heterocycloalkyl, aryl and heteroaryl is optionally substituted by one or more fluoro, R^{d} and R^{e};
with the proviso that (1) when Y⁹, Y⁸, Y⁷, or Y⁶ is —OMe, methyl, or fluoro, then at least one of Y⁹, Y⁸, Y⁷, Y⁶, Y⁵, Y⁴, Y³, Y², Y¹, R¹, or R^{c} is deuterium or is substituted with deuterium.

In certain embodiments of Formula I, Y⁷ is OR². Such compounds have formula II

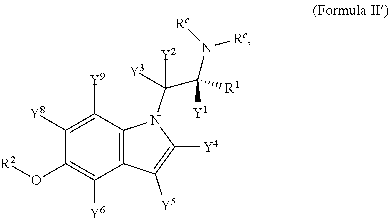

an enantiomer or diastereomer thereof, wherein
R² is selected from C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl and C₄₋₁₆ alkyl-heteroaryl; or Y⁶ and R² are combined with the atoms to which they are each attached to form a C₄₋₆ heterocycloalkyl or C₄₋₁₀ heteroaryl;
wherein each heterocycloalkyl and heteroaryl is optionally substituted by one or more fluoro, R^{d} and R^{e}.

In some embodiments of compounds of Formula II, or an enantiomer or diastereomer thereof are of Formula Ix, wherein
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁸ and Y⁹ are each independently selected from deuterium, hydrogen, halogen and C₁₋₆ alkyl,
R² is selected from haloalkyl and C₃₋₈ cycloalkyl, or R² and Y⁶ together form a C₄₋₁₀ heterocycloalkyl, or C₄₋₁₂ heteroaryl; and
R^{c} is, for each occurrence, selected from C₁₋₆ alkyl, C₃₋₈ cycloalkyl, or C₄₋₁₄ alkyl-cycloalkyl, or two of R^{c} and R¹ together with the atoms to which they are attached to form a C₂₋₁₂ heterocycloalkyl.

In other certain embodiments of Formula I, Y⁷ is SR², such compounds having formula III

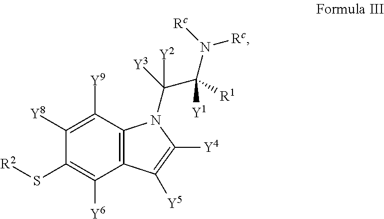

or
an enantiomer or diastereomer thereof wherein
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁸ and Y⁹ are each independently selected from deuterium, hydrogen, halogen and C₁₋₆ alkyl,
R² is selected from haloalkyl and C₃₋₈ cycloalkyl, or R² and Y⁶ together with the atoms to which they are attached form a C₄₋₆ heterocycloalkyl or C₄₋₁₀ heteroaryl;
wherein each heterocycloalkyl and heteroaryl is optionally substituted by one or more fluoro, R^{d} and R^{e}.

In some embodiments, the compound is of Formula IV:

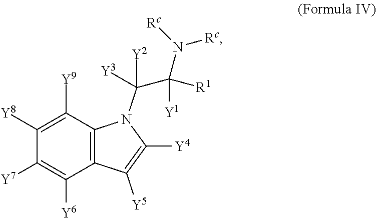

wherein Y¹ is hydrogen, deuterium, —CH₃, or —CD₃;
Y², Y³, Y⁴, Y⁵, Y⁸, and Y⁹ are each, independently, hydrogen or deuterium;
Y⁷ is:
   (i) —O—R², —S—R^{a}, —S(O)₂—R^{a}, —CN, -or S(F)₅;
      wherein R² is a C₃₋₈ cycloalkyl, CH₃, CD₃, or combines with Y⁶ to form a C₄₋₅ heterocycloalkyl; and
      R^{a} is a C₃₋₈ cycloalkyl or CH₃; or
   (ii) Y⁷ and Y⁶, together with the atoms to which they are attached, combine to form a C₆₋₁₀ aryl or a C₂₋₈ heteroaryl ring;
each R^{c} is, independently, CH₃ or CD₃;
R¹ is CH₃ or CD₃; and
Y⁶ is hydrogen, deuterium, or combines with R² to form a C₄₋₅ heterocycloalkyl or C₅₋₆ cycloalkyl;
with the proviso that when R² is CH₃, then at least one of Y¹, Y², Y³, Y⁴, Y⁵, Y⁸, and Y⁹ are deuterium, or at least one R_{c} is CD₃, or R₁ is CD₃;
or a pharmaceutically acceptable salt thereof.

In some embodiments, Y⁶ and Y⁷, together with the atoms to which they are attached, form a C₄₋₆ cycloalkyl, C₄₋₆ heterocycloalkyl, C₆₋₁₀ aryl, or C₄₋₁₀ heteroaryl. In some embodiments, Y⁷ is —O—R², —S—R^{a}, —S(O)₂—R^{a}, or —S(F)₅. In some embodiments, Y⁷ is —OCH₃, —OCD₃, —O-cyclopropyl, —S-cyclopropyl, or —S(O)₂-cyclopropyl.

In some embodiments, the compound is of Formula II′

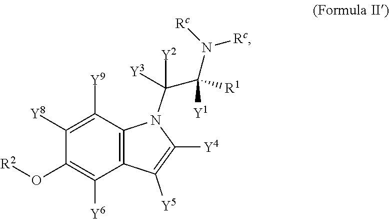

or
an enantiomer or diastereomer thereof, wherein
R² is selected from C₁₋₆ alkyl, C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl and C₄₋₁₆ alkyl-heteroaryl;
or a pharmaceutically acceptable salt thereof.

In some embodiments, R² is —CH₃, —CD₃, or cyclopropyl.

In some embodiments, the compound is of Formula Ix

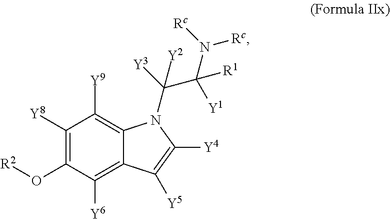

or
an enantiomer or diastereomer thereof wherein
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁸ and Y⁹ are each independently selected from hydrogen, deuterium, halogen and C₁₋₆ alkyl,
R² is selected from haloalkyl and C₃₋₈ cycloalkyl, or R² and Y⁶ together form a C₄₋₁₀ heterocycloalkyl, or C₄₋₁₂ heteroaryl; and
R^{c} is, for each occurrence, selected from C₁₋₆ alkyl, C₃₋₈ cycloalkyl, or C₄₋₁₄ alkyl-cycloalkyl, or two of R^{c} and R¹ together with the atoms to which they are attached to form a C₂₋₁₂ heterocycloalkyl;
or a pharmaceutically acceptable salt thereof.

In some embodiments, the compound is of Formula XIV:

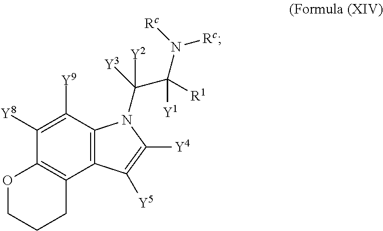

or the compound is of Formula XV:

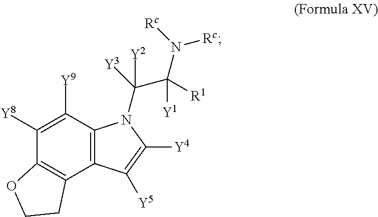

or a pharmaceutically acceptable salt thereof.

In some embodiments, each R^{c} is, independently, CH₃ or CD₃. In some embodiments, Y² and Y³ are each, independently, H or D. In some embodiments, R¹ is CH₃ or CD₃. In some embodiments, Y¹ is H, D, CH₃, or CD₃. In some embodiments, Y⁸, Y⁹, Y⁵, and Y⁴ are hydrogen. In some embodiments, the compound is of Formula III′:

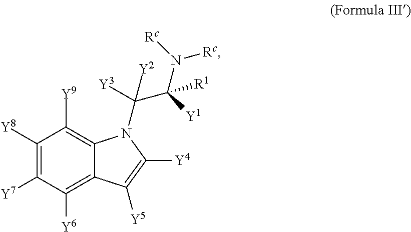

or
an enantiomer or diastereomer thereof wherein
R¹ is selected from C₁₋₆ alkyl, C₃₋₈ cycloalkyl, or C₄₋₁₄ alkyl-cycloalkyl;
Y¹, Y Y³, Y⁴, Y⁵, Y⁶, Y⁸ and Y⁹ are each independently selected from hydrogen, deuterium, halogen and C₁₋₆ alkyl,
Y⁷ is selected from —S(F)₅ or —S—R².
R² is selected from CH₃ or C₃₋₈ cycloalkyl, or R² and Y⁶ together form a C₄₋₁₀ heterocycloalkyl, or C₄₋₁₂ heteroaryl; and
R^{c} is, for each occurrence, selected from C₁₋₆ alkyl, C₃₋₈ cycloalkyl, or C₄₋₁₄ alkyl-cycloalkyl, or two of R^{c} and R¹ together with the atoms to which they are attached to form a C₂₋₁₂ heterocycloalkyl;
or a pharmaceutically acceptable salt thereof.

In some embodiments, R² is cyclopropyl.

In some embodiments, the compound is of Formula V:

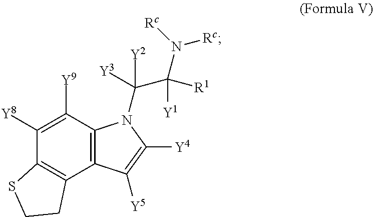

or wherein the compound is of Formula VI:

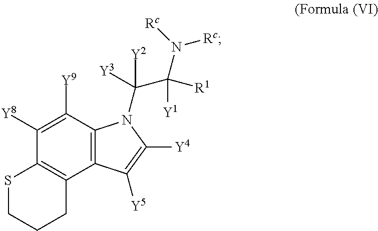

or a pharmaceutically acceptable salt thereof, wherein
each R^{c} is methyl;
Y¹ is H or methyl;
R¹ is methyl; and
Y², Y³, Y⁴, Y⁵, Y⁸, and Y⁹ are hydrogen.

In some embodiments, the compound is of Formula VII:

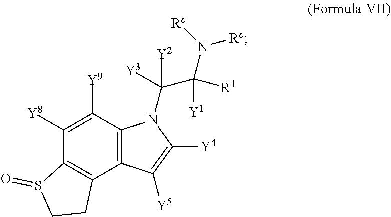

or the compound is of Formula VIII:

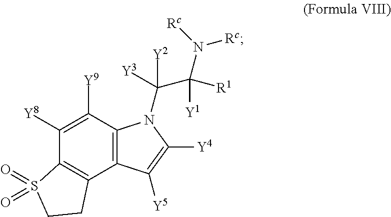

or the compound is of Formula IX:

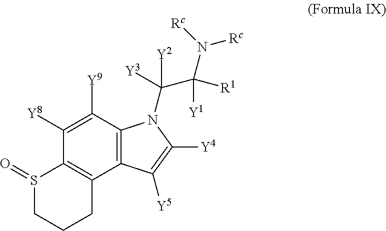

or the compound is of Formula X:

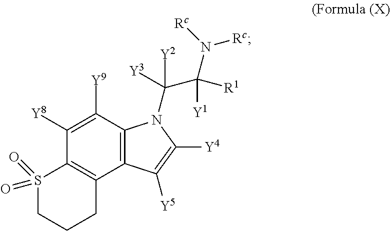

or a pharmaceutically acceptable salt thereof;
wherein each R^{c} is methyl;
   Y¹ is H or methyl;
   R¹ is methyl; and
      Y², Y³, Y⁴, Y⁵, Y⁸, and Y⁹ are hydrogen.

In some embodiments, the compound is of Formula XI:

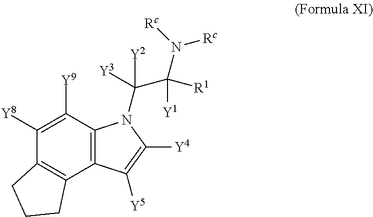

or the compound is of Formula XII:

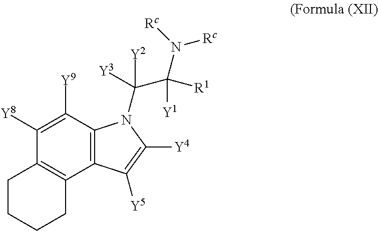

or the compound is of Formula XIII:

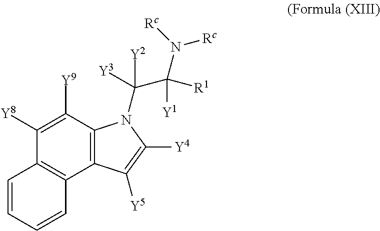

or a pharmaceutically acceptable salt thereof;
wherein each R^{c} is methyl;
Y¹ is H or methyl;
R¹ is methyl; and
   Y², Y³, Y⁴, Y⁵, Y⁸, and Y⁹ are hydrogen.

In some embodiments of the proceeding compounds, at least one of Y¹, Y², Y³, Y⁴, Y⁵, Y⁶, Y⁷, Y⁸ and Y⁹ is deuterium. In some embodiments, at least one R^{c} is deuterium. In some embodiments, at least one of R¹, R² and R^{c} is deuterium.

In some embodiments, the compound of Formula IA is of any one of Formula IA-i, Formula IA-ii, Formula IA-iii, Formula IA-iv, or Formula IA-v:

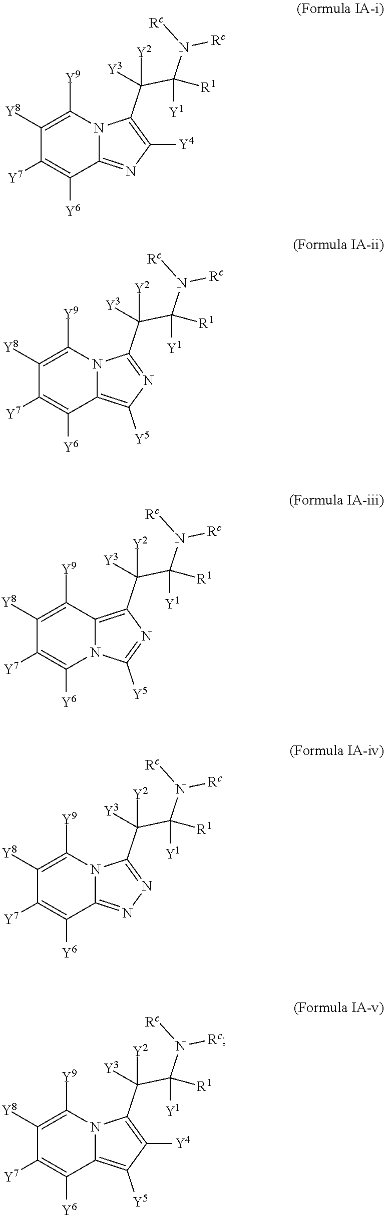

or a pharmaceutically acceptable salt thereof.

In some embodiments of any one of Formulae IA-i, IA-ii, IA-iii, IA-iv, and/or IA-v:
Y¹, Y², Y³, Y⁴, Y⁵, Y⁶, Y⁸, and Y⁹ are each independently hydrogen or deuterium;
Y⁷ is halo, e.g., fluoro, or —O—C₁-C₆ alkyl, e.g., —O—CH₃, wherein the alkyl group is optionally substituted with deuterium, e.g., —CD₃, CHD₂, or CH₂D;
R^{c} is a C₁-C₆ alkyl optionally substituted with deuterium, e.g., a methyl group optionally substituted with deuterium; and
R¹ is a C₁-C₆ alkyl optionally substituted with deuterium, e.g., a methyl group optionally substituted with deuterium.

Particular examples of the compounds of Formulas I, II and III include the following compounds:

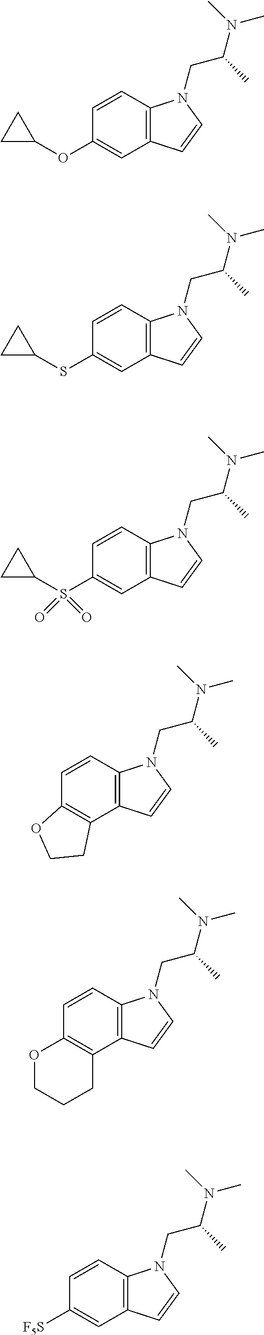

or a stereoisomer, enantiomer or diastereomer thereof.

Other particular examples of the compounds of Formulas I, II and III include those having the formula

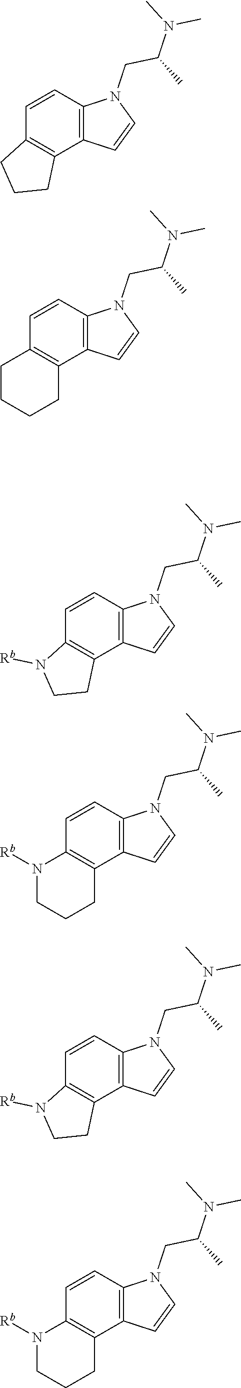

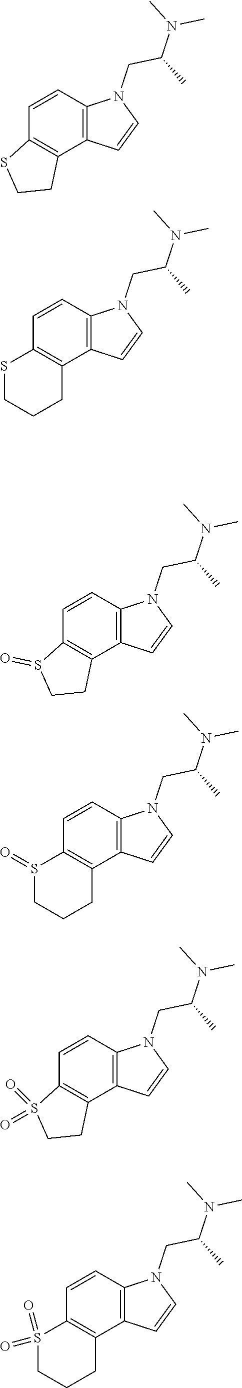 [SMILES: C[C@H](Cn1ccc2c3c(ccc21)SCC3)N(C)C ; C[C@H](Cn1ccc2c3c(ccc21)SCCC3)N(C)C ; C[C@H](Cn1ccc2c3c(ccc21)S(=O)CC3)N(C)C ; C[C@H](Cn1ccc2c3c(ccc21)S(=O)CCC3)N(C)C ; C[C@H](Cn1ccc2c3c(ccc21)S(=O)(=O)CC3)N(C)C ; C[C@H](Cn1ccc2c3c(ccc21)S(=O)(=O)CCC3)N(C)C]

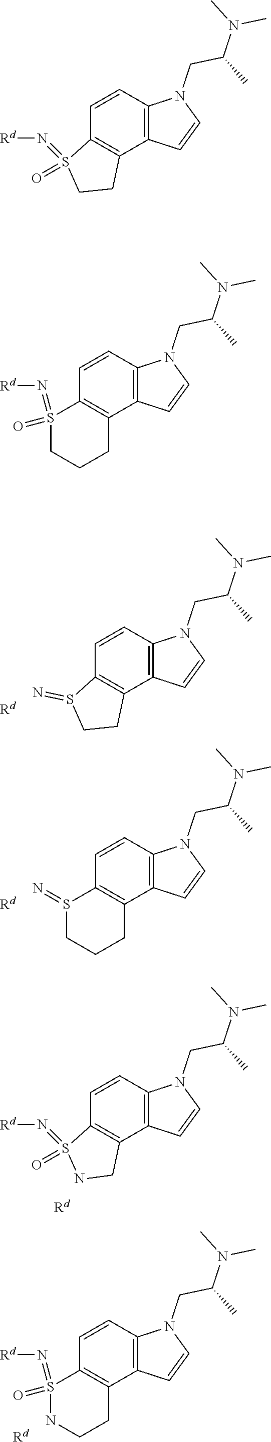

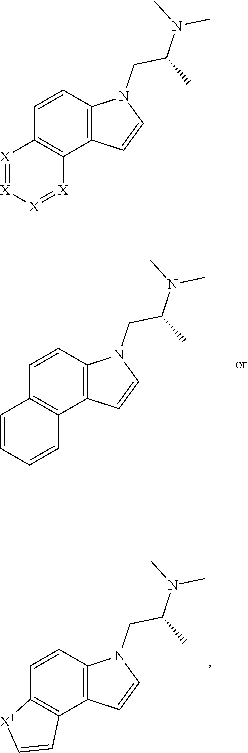

wherein
X is, independently for each occurrence, CH or N;
X¹ is selected from O, S, NR and NR^{c}; or
   an enantiomer or diastereomer thereof.

Particular examples of compounds having Formula I or II include those of the formula

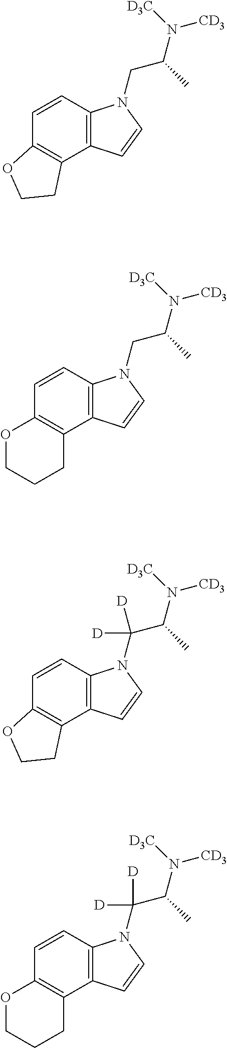 [SMILES: [2H]C([2H])([2H])N([C@H](C)Cn1ccc2c3c(ccc21)OCC3)C([2H])([2H])[2H] ; [2H]C([2H])([2H])N([C@H](C)Cn1ccc2c3c(ccc21)OCCC3)C([2H])([2H])[2H] ; [2H]C([2H])([2H])N([C@H](C)C([2H])([2H])n1ccc2c3c(ccc21)OCC3)C([2H])([2H])[2H] ; [2H]C([2H])([2H])N([C@H](C)C([2H])([2H])n1ccc2c3c(ccc21)OCCC3)C([2H])([2H])[2H]]

an enantiomer or diastereomer thereof.

In some embodiments, the present disclosure provides any one of the compounds in Table 1:

TABLE 1

| Compound | |
|---|---|
| Number | Structure |
| 1 | 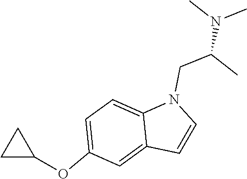 [SMILES: C[C@H](Cn1ccc2cc(OC3CC3)ccc21)N(C)C] |
| 2 | 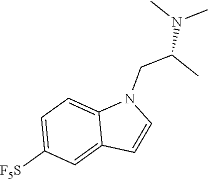 |
| 3 | 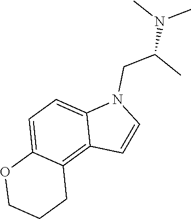 [SMILES: C[C@H](Cn1ccc2c3c(ccc21)OCCC3)N(C)C] |
| 4 | 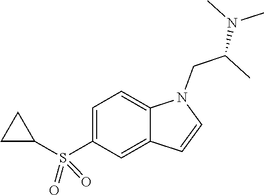 [SMILES: C[C@H](Cn1ccc2cc(S(=O)(=O)C3CC3)ccc21)N(C)C] |
| 5 | 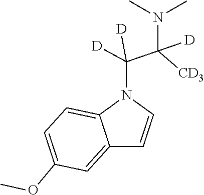 [SMILES: [2H]C([2H])([2H])C([2H])(N(C)C)C([2H])([2H])n1ccc2cc(OC)ccc21] |
| 6 | 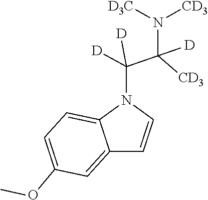 [SMILES: [2H]C([2H])([2H])N(C([2H])([2H])[2H])C([2H])(C([2H])([2H])[2H])C([2H])([2H])n1ccc2cc(OC)ccc21] |
| 7 | 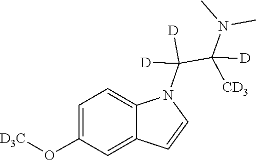 [SMILES: [2H]C([2H])([2H])Oc1ccc2c(ccn2C([2H])([2H])C([2H])(N(C)C)C([2H])([2H])[2H])c1] |
| 8 | 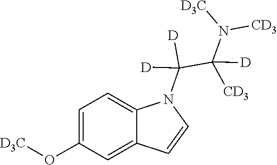 [SMILES: [2H]C([2H])([2H])Oc1ccc2c(ccn2C([2H])([2H])C([2H])(N(C([2H])([2H])[2H])C([2H])([2H])[2H])C([2H])([2H])[2H])c1] |
| 9 | 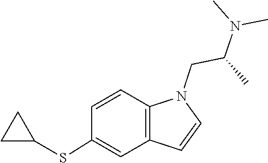 [SMILES: C[C@H](Cn1ccc2cc(SC3CC3)ccc21)N(C)C] |
| 10 | 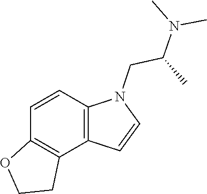 [SMILES: C[C@H](Cn1ccc2c3c(ccc21)OCC3)N(C)C] |
| 11 | 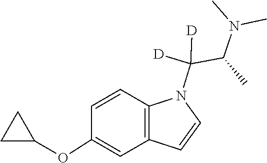 [SMILES: [2H]C([2H])([C@@H](C)N(C)C)n1ccc2cc(OC3CC3)ccc21] |
| 12 | 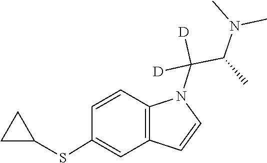 [SMILES: [2H]C([2H])([C@@H](C)N(C)C)n1ccc2cc(SC3CC3)ccc21] |
| 13 | 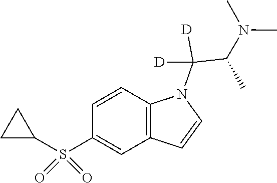 [SMILES: [2H]C([2H])([C@@H](C)N(C)C)n1ccc2cc(S(=O)(=O)C3CC3)ccc21] |
| 14 | 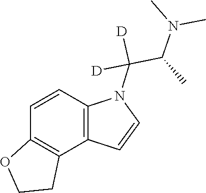 [SMILES: [2H]C([2H])([C@@H](C)N(C)C)n1ccc2c3c(ccc21)OCC3] |
| 15 | 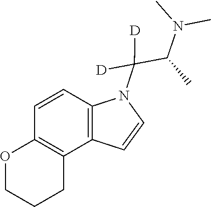 [SMILES: [2H]C([2H])([C@@H](C)N(C)C)n1ccc2c3c(ccc21)OCCC3] |
| 16 | 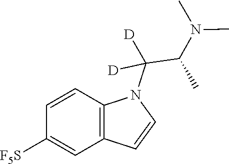 |
| 17 | 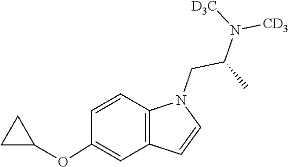 [SMILES: [2H]C([2H])([2H])N([C@H](C)Cn1ccc2cc(OC3CC3)ccc21)C([2H])([2H])[2H]] |
| 18 | 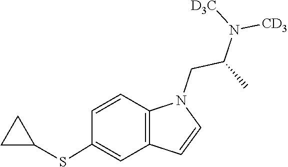 [SMILES: [2H]C([2H])([2H])N([C@H](C)Cn1ccc2cc(SC3CC3)ccc21)C([2H])([2H])[2H]] |
| 19 | 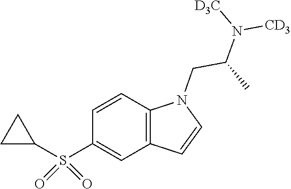 [SMILES: [2H]C([2H])([2H])N([C@H](C)Cn1ccc2cc(S(=O)(=O)C3CC3)ccc21)C([2H])([2H])[2H]] |
| 20 | 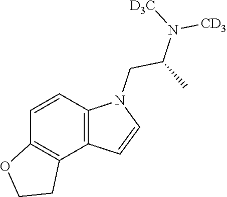 [SMILES: [2H]C([2H])([2H])N([C@H](C)Cn1ccc2c3c(ccc21)OCC3)C([2H])([2H])[2H]] |
| 21 | 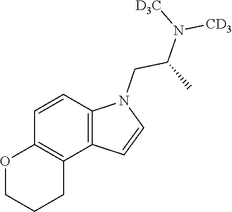 [SMILES: [2H]C([2H])([2H])N([C@H](C)Cn1ccc2c3c(ccc21)OCCC3)C([2H])([2H])[2H]] |
| 22 | 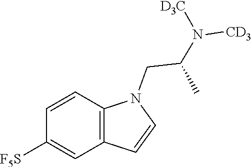 |
| 23 | 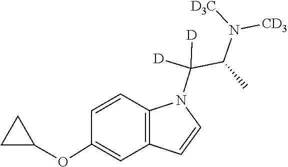 [SMILES: [2H]C([2H])([2H])N([C@H](C)C([2H])([2H])n1ccc2cc(OC3CC3)ccc21)C([2H])([2H])[2H]] |
| 24 | 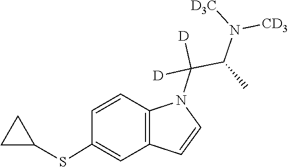 [SMILES: [2H]C([2H])([2H])N([C@H](C)C([2H])([2H])n1ccc2cc(SC3CC3)ccc21)C([2H])([2H])[2H]] |
| 25 | 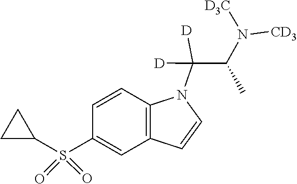 [SMILES: [2H]C([2H])([2H])N([C@H](C)C([2H])([2H])n1ccc2cc(S(=O)(=O)C3CC3)ccc21)C([2H])([2H])[2H]] |
| 26 | 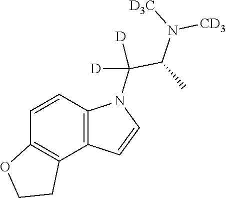 [SMILES: [2H]C([2H])([2H])N([C@H](C)C([2H])([2H])n1ccc2c3c(ccc21)OCC3)C([2H])([2H])[2H]] |
| 27 | 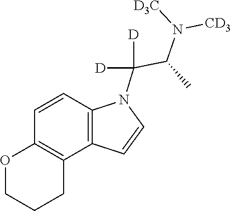 [SMILES: [2H]C([2H])([2H])N([C@H](C)C([2H])([2H])n1ccc2c3c(ccc21)OCCC3)C([2H])([2H])[2H]] |
| 28 | 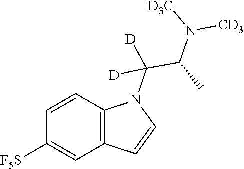 |
| 29 | 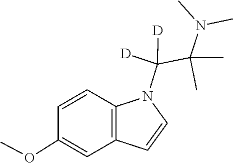 |
| 30 | 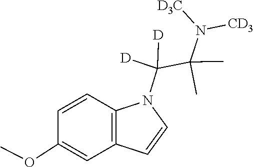 [SMILES: [2H]C([2H])([2H])N(C([2H])([2H])[2H])C(C)(C)C([2H])([2H])n1ccc2cc(OC)ccc21] |
| 31 | 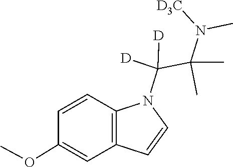 |
| 32 | 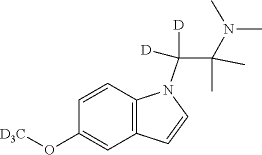 [SMILES: [2H]C([2H])([2H])Oc1ccc2c(ccn2C([2H])([2H])C(C)(C)N(C)C)c1] |
| 33 | 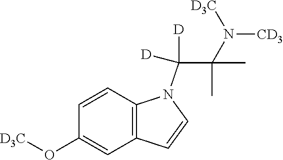 [SMILES: [2H]C([2H])([2H])Oc1ccc2c(ccn2C([2H])([2H])C(C)(C)N(C([2H])([2H])[2H])C([2H])([2H])[2H])c1] |
| 34 | 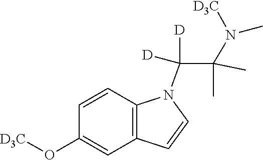 [SMILES: [2H]C([2H])([2H])Oc1ccc2c(ccn2C([2H])([2H])C(C)(C)N(C)C([2H])([2H])[2H])c1] |
| 35 | 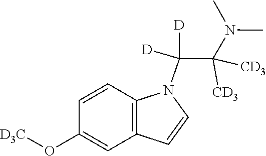 [SMILES: [2H]C([2H])([2H])Oc1ccc2c(ccn2C([2H])([2H])C(N(C)C)(C([2H])([2H])[2H])C([2H])([2H])[2H])c1] |
| 36 | 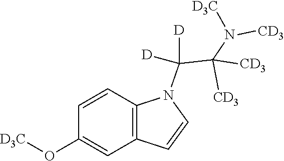 [SMILES: [2H]C([2H])([2H])Oc1ccc2c(ccn2C([2H])([2H])C(N(C([2H])([2H])[2H])C([2H])([2H])[2H])(C([2H])([2H])[2H])C([2H])([2H])[2H])c1] |
| 37 | 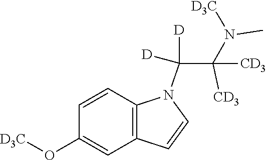 [SMILES: [2H]C([2H])([2H])Oc1ccc2c(ccn2C([2H])([2H])C(N(C)C([2H])([2H])[2H])(C([2H])([2H])[2H])C([2H])([2H])[2H])c1] |
| 38 | 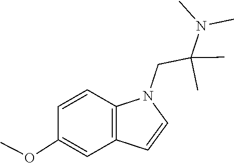 |
| 39 | 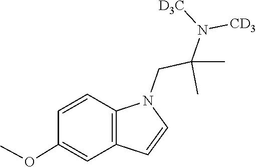 [SMILES: [2H]C([2H])([2H])N(C([2H])([2H])[2H])C(C)(C)Cn1ccc2cc(OC)ccc21] |
| 40 | 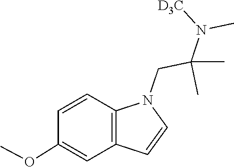 |
| 41 | 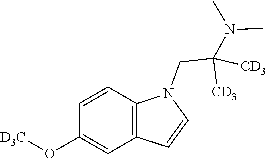 [SMILES: [2H]C([2H])([2H])Oc1ccc2c(ccn2CC(N(C)C)(C([2H])([2H])[2H])C([2H])([2H])[2H])c1] |
| 42 | 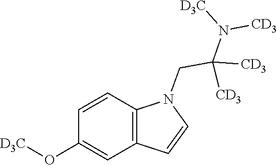 [SMILES: [2H]C([2H])([2H])Oc1ccc2c(ccn2CC(N(C([2H])([2H])[2H])C([2H])([2H])[2H])(C([2H])([2H])[2H])C([2H])([2H])[2H])c1] |
| 43 | 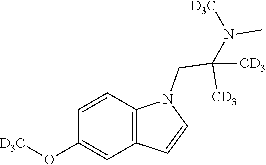 [SMILES: [2H]C([2H])([2H])Oc1ccc2c(ccn2CC(N(C)C([2H])([2H])[2H])(C([2H])([2H])[2H])C([2H])([2H])[2H])c1] |
| 44 | 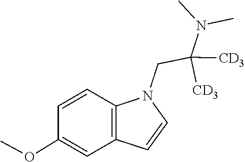 |
| 45 | 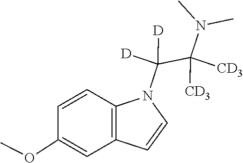 |
| 46 | 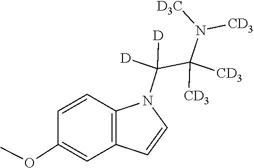 [SMILES: [2H]C([2H])([2H])N(C([2H])([2H])[2H])C(C([2H])([2H])[2H])(C([2H])([2H])[2H])C([2H])([2H])n1ccc2cc(OC)ccc21] |
| 47 | 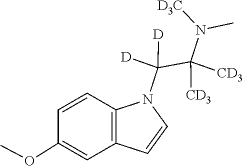 |
| 48 | 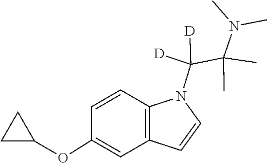 [SMILES: [2H]C([2H])(n1ccc2cc(OC3CC3)ccc21)C(C)(C)N(C)C] |
| 49 | 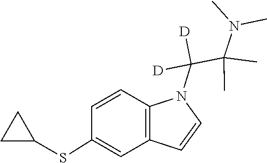 [SMILES: [2H]C([2H])(n1ccc2cc(SC3CC3)ccc21)C(C)(C)N(C)C] |
| 50 | 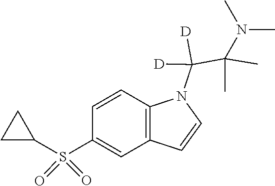 [SMILES: [2H]C([2H])(n1ccc2cc(S(=O)(=O)C3CC3)ccc21)C(C)(C)N(C)C] |
| 51 | 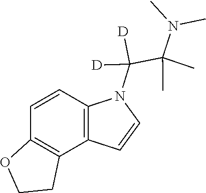 [SMILES: [2H]C([2H])(n1ccc2c3c(ccc21)OCC3)C(C)(C)N(C)C] |
| 52 | 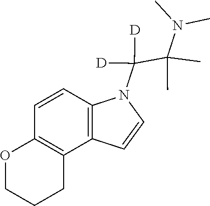 [SMILES: [2H]C([2H])(n1ccc2c3c(ccc21)OCCC3)C(C)(C)N(C)C] |
| 53 | 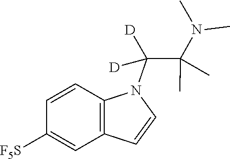 |
| 54 | 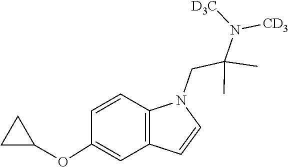 [SMILES: [2H]C([2H])([2H])N(C([2H])([2H])[2H])C(C)(C)Cn1ccc2cc(OC3CC3)ccc21] |
| 55 | 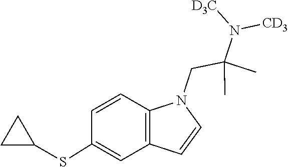 [SMILES: [2H]C([2H])([2H])N(C([2H])([2H])[2H])C(C)(C)Cn1ccc2cc(SC3CC3)ccc21] |
| 56 | 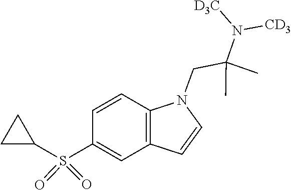 [SMILES: [2H]C([2H])([2H])N(C([2H])([2H])[2H])C(C)(C)Cn1ccc2cc(S(=O)(=O)C3CC3)ccc21] |
| 57 | 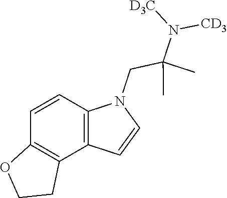 [SMILES: [2H]C([2H])([2H])N(C([2H])([2H])[2H])C(C)(C)Cn1ccc2c3c(ccc21)OCC3] |
| 58 | 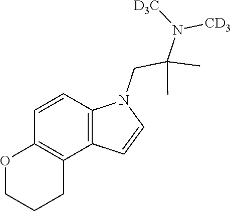 [SMILES: [2H]C([2H])([2H])N(C([2H])([2H])[2H])C(C)(C)Cn1ccc2c3c(ccc21)OCCC3] |
| 59 | 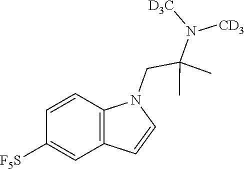 |
| 60 | 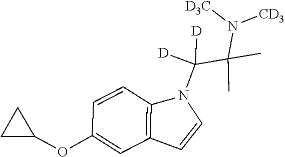 [SMILES: [2H]C([2H])([2H])N(C([2H])([2H])[2H])C(C)(C)C([2H])([2H])n1ccc2cc(OC3CC3)ccc21] |
| 61 | 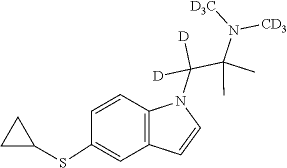 [SMILES: [2H]C([2H])([2H])N(C([2H])([2H])[2H])C(C)(C)C([2H])([2H])n1ccc2cc(SC3CC3)ccc21] |
| 62 | 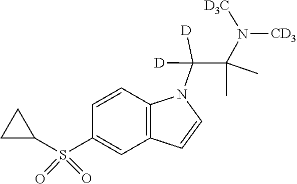 [SMILES: [2H]C([2H])([2H])N(C([2H])([2H])[2H])C(C)(C)C([2H])([2H])n1ccc2cc(S(=O)(=O)C3CC3)ccc21] |
| 63 | 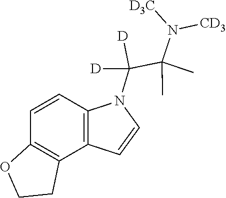 [SMILES: [2H]C([2H])([2H])N(C([2H])([2H])[2H])C(C)(C)C([2H])([2H])n1ccc2c3c(ccc21)OCC3] |
| 64 | 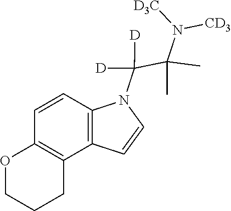 [SMILES: [2H]C([2H])([2H])N(C([2H])([2H])[2H])C(C)(C)C([2H])([2H])n1ccc2c3c(ccc21)OCCC3] |
| 65 | 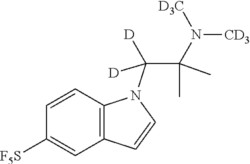 |
| 66 | 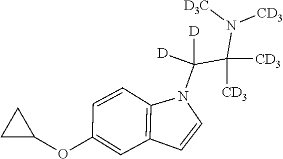 [SMILES: [2H]C([2H])([2H])N(C([2H])([2H])[2H])C(C([2H])([2H])[2H])(C([2H])([2H])[2H])C([2H])([2H])n1ccc2cc(OC3CC3)ccc21] |
| 67 | 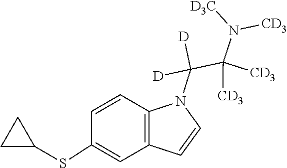 [SMILES: [2H]C([2H])([2H])N(C([2H])([2H])[2H])C(C([2H])([2H])[2H])(C([2H])([2H])[2H])C([2H])([2H])n1ccc2cc(SC3CC3)ccc21] |
| 68 | 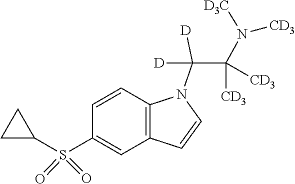 [SMILES: [2H]C([2H])([2H])N(C([2H])([2H])[2H])C(C([2H])([2H])[2H])(C([2H])([2H])[2H])C([2H])([2H])n1ccc2cc(S(=O)(=O)C3CC3)ccc21] |
| 69 | 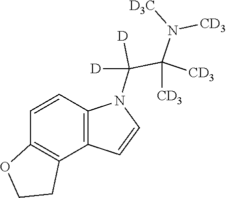 [SMILES: [2H]C([2H])([2H])N(C([2H])([2H])[2H])C(C([2H])([2H])[2H])(C([2H])([2H])[2H])C([2H])([2H])n1ccc2c3c(ccc21)OCC3] |
| 70 | 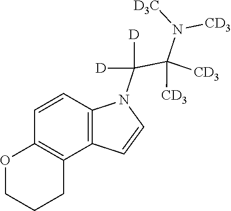 [SMILES: [2H]C([2H])([2H])N(C([2H])([2H])[2H])C(C([2H])([2H])[2H])(C([2H])([2H])[2H])C([2H])([2H])n1ccc2c3c(ccc21)OCCC3] |
| 71 | 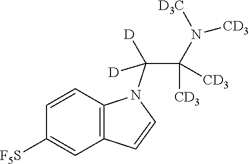 |
| 72 | 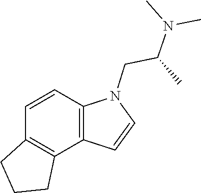 [SMILES: C[C@H](Cn1ccc2c3c(ccc21)CCC3)N(C)C] |
| 73 | 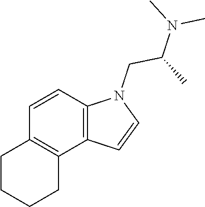 [SMILES: C[C@H](Cn1ccc2c3c(ccc21)CCCC3)N(C)C] |
| 74 | 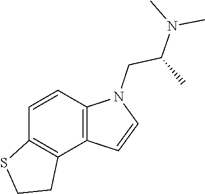 [SMILES: C[C@H](Cn1ccc2c3c(ccc21)SCC3)N(C)C] |
| 75 | 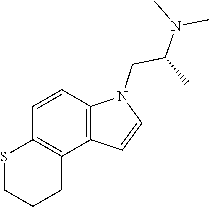 [SMILES: C[C@H](Cn1ccc2c3c(ccc21)SCCC3)N(C)C] |
| 76 | 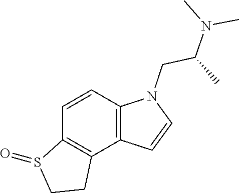 [SMILES: C[C@H](Cn1ccc2c3c(ccc21)S(=O)CC3)N(C)C] |
| 77 | 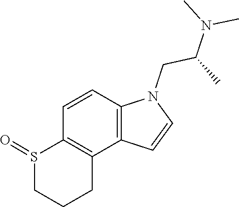 [SMILES: C[C@H](Cn1ccc2c3c(ccc21)S(=O)CCC3)N(C)C] |
| 78 | 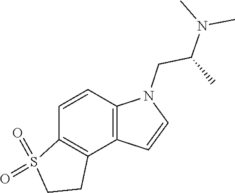 [SMILES: C[C@H](Cn1ccc2c3c(ccc21)S(=O)(=O)CC3)N(C)C] |
| 79 | 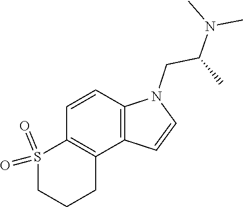 [SMILES: C[C@H](Cn1ccc2c3c(ccc21)S(=O)(=O)CCC3)N(C)C] |
| 80 | 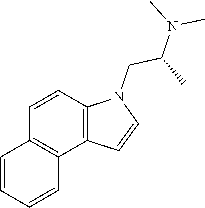 [SMILES: C[C@H](Cn1ccc2c3ccccc3ccc21)N(C)C] |
| 81 | 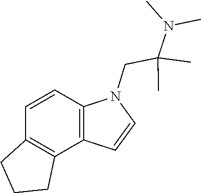 [SMILES: CN(C)C(C)(C)Cn1ccc2c3c(ccc21)CCC3] |
| 82 | 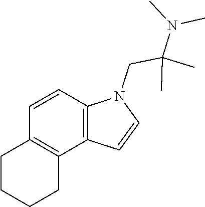 [SMILES: CN(C)C(C)(C)Cn1ccc2c3c(ccc21)CCCC3] |
| 83 | 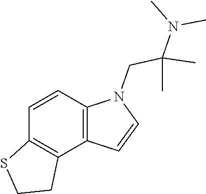 [SMILES: CN(C)C(C)(C)Cn1ccc2c3c(ccc21)SCC3] |
| 84 | 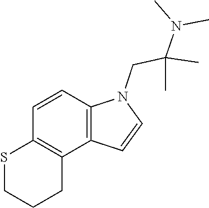 [SMILES: CN(C)C(C)(C)Cn1ccc2c3c(ccc21)SCCC3] |
| 85 | 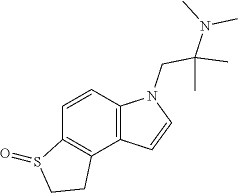 [SMILES: CN(C)C(C)(C)Cn1ccc2c3c(ccc21)S(=O)CC3] |
| 86 | 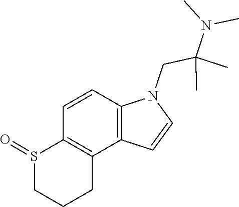 [SMILES: CN(C)C(C)(C)Cn1ccc2c3c(ccc21)S(=O)CCC3] |
| 87 | 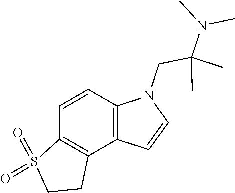 [SMILES: CN(C)C(C)(C)Cn1ccc2c3c(ccc21)S(=O)(=O)CC3] |
| 88 | 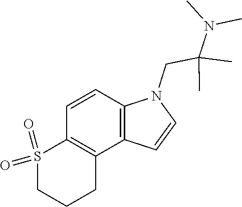 [SMILES: CN(C)C(C)(C)Cn1ccc2c3c(ccc21)S(=O)(=O)CCC3] |
| 89 | 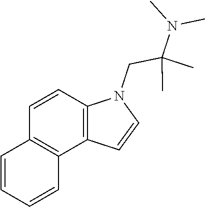 [SMILES: CN(C)C(C)(C)Cn1ccc2c3ccccc3ccc21] |
| 90 | 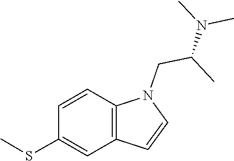 |
| 91 | 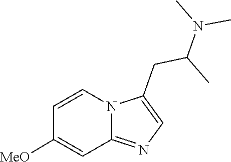 [SMILES: COc1ccn2c(CC(C)N(C)C)cnc2c1] |
| 92 | 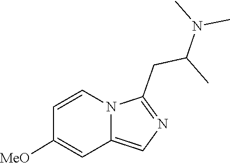 [SMILES: COc1ccn2c(CC(C)N(C)C)ncc2c1] |
| 93 | 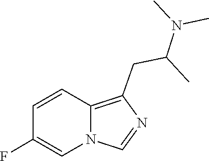 [SMILES: CC(Cc1ncn2cc(F)ccc12)N(C)C] |
| 94 | 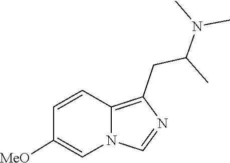 [SMILES: COc1ccc2c(CC(C)N(C)C)ncn2c1] |
| 95 | 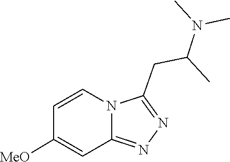 [SMILES: COc1ccn2c(CC(C)N(C)C)nnc2c1] |
| 96 | 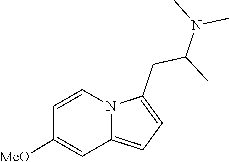 [SMILES: COc1ccn2c(CC(C)N(C)C)ccc2c1] |
| 97 | 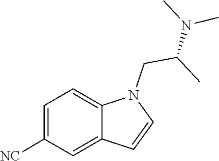 [SMILES: C[C@H](Cn1ccc2cc(C#N)ccc21)N(C)C] |
| 98 | 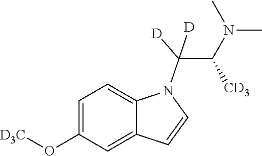 [SMILES: [2H]C([2H])([2H])Oc1ccc2c(ccn2C([2H])([2H])[C@H](N(C)C)C([2H])([2H])[2H])c1] |
| 99 | 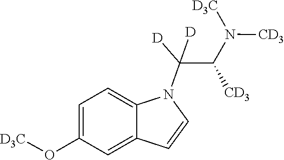 [SMILES: [2H]C([2H])([2H])Oc1ccc2c(ccn2C([2H])([2H])[C@H](N(C([2H])([2H])[2H])C([2H])([2H])[2H])C([2H])([2H])[2H])c1] |
| 100 | 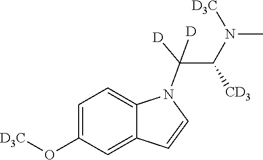 [SMILES: [2H]C([2H])([2H])Oc1ccc2c(ccn2C([2H])([2H])[C@H](N(C)C([2H])([2H])[2H])C([2H])([2H])[2H])c1] |
| 101 | 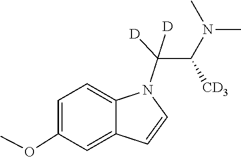 |
| 102 | 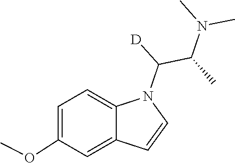 |
| 103 | 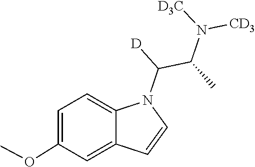 [SMILES: [2H]C([C@@H](C)N(C([2H])([2H])[2H])C([2H])([2H])[2H])n1ccc2cc(OC)ccc21] |
| 104 | 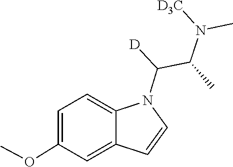 |
| 105 | 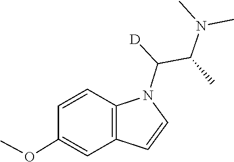 |
| 106 | 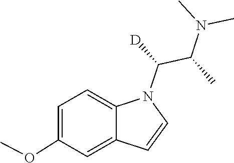 |
| 107 | 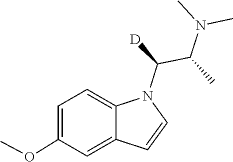 |
| 108 | 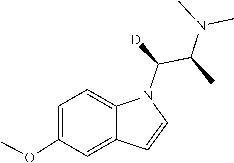 |
| 109 | 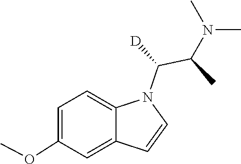 |
| 110 | 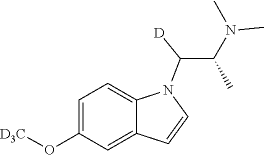 [SMILES: [2H]C([C@@H](C)N(C)C)n1ccc2cc(OC([2H])([2H])[2H])ccc21] |
| 111 | 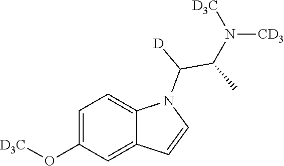 [SMILES: [2H]C([C@@H](C)N(C([2H])([2H])[2H])C([2H])([2H])[2H])n1ccc2cc(OC([2H])([2H])[2H])ccc21] |
| 112 | 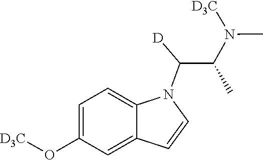 [SMILES: [2H]C([C@@H](C)N(C)C([2H])([2H])[2H])n1ccc2cc(OC([2H])([2H])[2H])ccc21] |
| 113 | 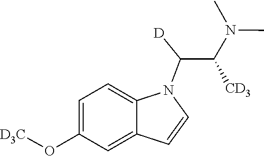 [SMILES: [2H]C([C@H](N(C)C)C([2H])([2H])[2H])n1ccc2cc(OC([2H])([2H])[2H])ccc21] |
| 114 | 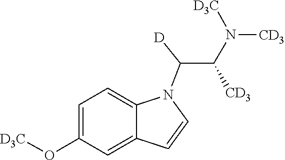 [SMILES: [2H]C([C@H](N(C([2H])([2H])[2H])C([2H])([2H])[2H])C([2H])([2H])[2H])n1ccc2cc(OC([2H])([2H])[2H])ccc21] |
| 115 | 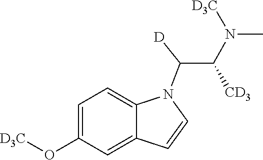 [SMILES: [2H]C([C@H](N(C)C([2H])([2H])[2H])C([2H])([2H])[2H])n1ccc2cc(OC([2H])([2H])[2H])ccc21] |
| 116 | 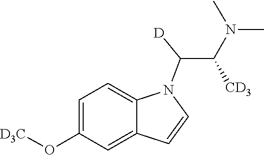 [SMILES: [2H]C([C@H](N(C)C)C([2H])([2H])[2H])n1ccc2cc(OC([2H])([2H])[2H])ccc21] |
| 117 | 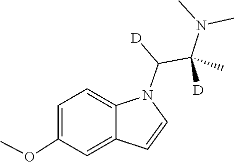 |
| 118 | 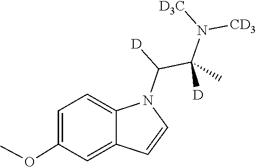 [SMILES: [2H]C(n1ccc2cc(OC)ccc21)[C@@]([2H])(C)N(C([2H])([2H])[2H])C([2H])([2H])[2H]] |
| 119 | 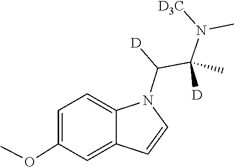 |
| 120 | 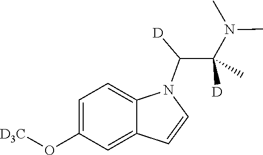 [SMILES: [2H]C(n1ccc2cc(OC([2H])([2H])[2H])ccc21)[C@@]([2H])(C)N(C)C] |
| 121 | 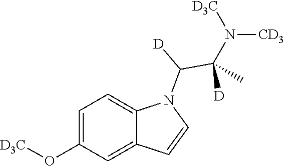 [SMILES: [2H]C(n1ccc2cc(OC([2H])([2H])[2H])ccc21)[C@@]([2H])(C)N(C([2H])([2H])[2H])C([2H])([2H])[2H]] |
| 122 | 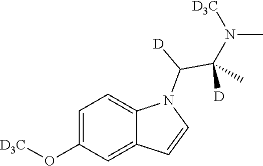 [SMILES: [2H]C(n1ccc2cc(OC([2H])([2H])[2H])ccc21)[C@@]([2H])(C)N(C)C([2H])([2H])[2H]] |
| 123 | 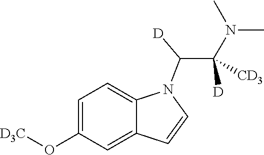 [SMILES: [2H]C(n1ccc2cc(OC([2H])([2H])[2H])ccc21)[C@]([2H])(N(C)C)C([2H])([2H])[2H]] |
| 124 | 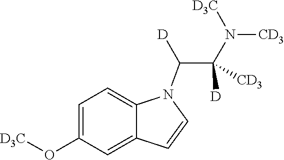 [SMILES: [2H]C(n1ccc2cc(OC([2H])([2H])[2H])ccc21)[C@]([2H])(N(C([2H])([2H])[2H])C([2H])([2H])[2H])C([2H])([2H])[2H]] |
| 125 | 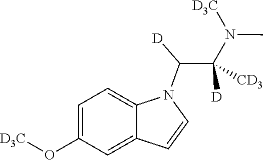 [SMILES: [2H]C(n1ccc2cc(OC([2H])([2H])[2H])ccc21)[C@]([2H])(N(C)C([2H])([2H])[2H])C([2H])([2H])[2H]] |
| 126 | 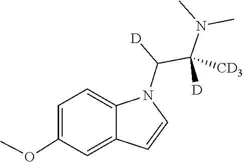 |
| 127 | 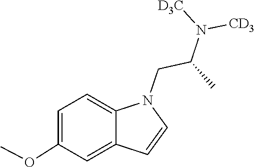 [SMILES: [2H]C([2H])([2H])N([C@H](C)Cn1ccc2cc(OC)ccc21)C([2H])([2H])[2H]] |
| 128 | 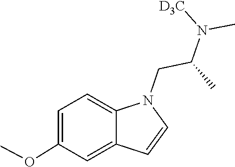 |
| 129 | 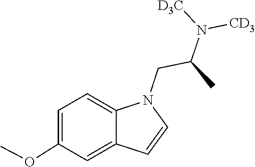 [SMILES: [2H]C([2H])([2H])N([C@@H](C)Cn1ccc2cc(OC)ccc21)C([2H])([2H])[2H]] |
| 130 | 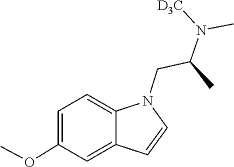 |
| 131 | 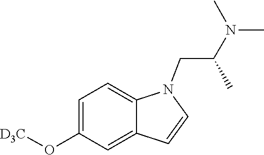 [SMILES: [2H]C([2H])([2H])Oc1ccc2c(ccn2C[C@@H](C)N(C)C)c1] |
| 132 | 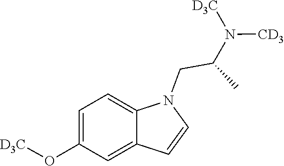 [SMILES: [2H]C([2H])([2H])Oc1ccc2c(ccn2C[C@@H](C)N(C([2H])([2H])[2H])C([2H])([2H])[2H])c1] |
| 133 | 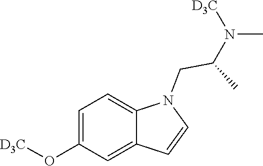 [SMILES: [2H]C([2H])([2H])Oc1ccc2c(ccn2C[C@@H](C)N(C)C([2H])([2H])[2H])c1] |
| 134 | 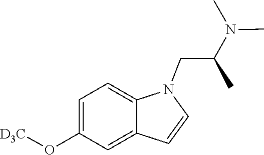 [SMILES: [2H]C([2H])([2H])Oc1ccc2c(ccn2C[C@H](C)N(C)C)c1] |
| 135 | 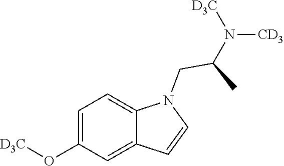 [SMILES: [2H]C([2H])([2H])Oc1ccc2c(ccn2C[C@H](C)N(C([2H])([2H])[2H])C([2H])([2H])[2H])c1] |
| 136 | 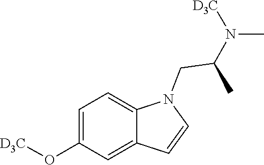 [SMILES: [2H]C([2H])([2H])Oc1ccc2c(ccn2C[C@H](C)N(C)C([2H])([2H])[2H])c1] |
| 137 | 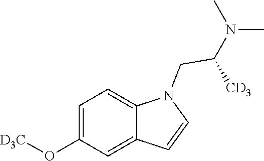 [SMILES: [2H]C([2H])([2H])Oc1ccc2c(ccn2C[C@H](N(C)C)C([2H])([2H])[2H])c1] |
| 138 | 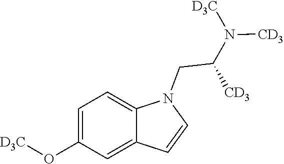 [SMILES: [2H]C([2H])([2H])Oc1ccc2c(ccn2C[C@H](N(C([2H])([2H])[2H])C([2H])([2H])[2H])C([2H])([2H])[2H])c1] |
| 139 | 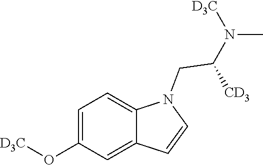 [SMILES: [2H]C([2H])([2H])Oc1ccc2c(ccn2C[C@H](N(C)C([2H])([2H])[2H])C([2H])([2H])[2H])c1] |
| 140 | 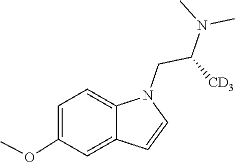 |
| 141 | 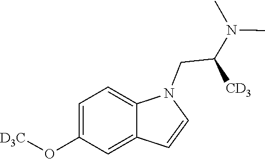 [SMILES: [2H]C([2H])([2H])Oc1ccc2c(ccn2C[C@@H](N(C)C)C([2H])([2H])[2H])c1] |
| 142 | 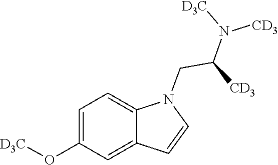 [SMILES: [2H]C([2H])([2H])Oc1ccc2c(ccn2C[C@@H](N(C([2H])([2H])[2H])C([2H])([2H])[2H])C([2H])([2H])[2H])c1] |
| 143 | 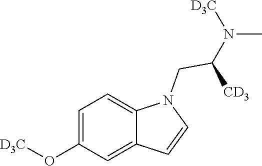 [SMILES: [2H]C([2H])([2H])Oc1ccc2c(ccn2C[C@@H](N(C)C([2H])([2H])[2H])C([2H])([2H])[2H])c1] |
| 144 | 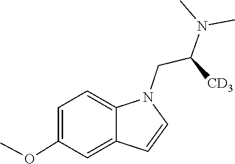 |
| 145 | 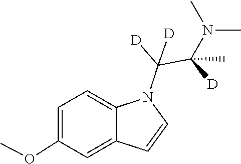 |
| 146 | 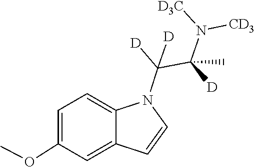 [SMILES: [2H]C([2H])([2H])N(C([2H])([2H])[2H])[C@]([2H])(C)C([2H])([2H])n1ccc2cc(OC)ccc21] |
| 147 | 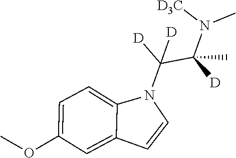 |
| 148 | 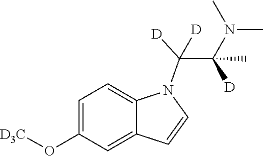 [SMILES: [2H]C([2H])([2H])Oc1ccc2c(ccn2C([2H])([2H])[C@@]([2H])(C)N(C)C)c1] |
| 149 | 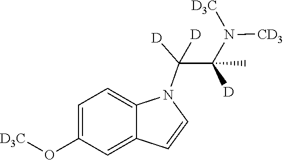 [SMILES: [2H]C([2H])([2H])Oc1ccc2c(ccn2C([2H])([2H])[C@@]([2H])(C)N(C([2H])([2H])[2H])C([2H])([2H])[2H])c1] |
| 150 | 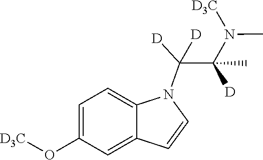 [SMILES: [2H]C([2H])([2H])Oc1ccc2c(ccn2C([2H])([2H])[C@@]([2H])(C)N(C)C([2H])([2H])[2H])c1] |
| 151 | 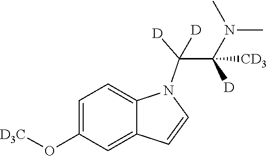 [SMILES: [2H]C([2H])([2H])Oc1ccc2c(ccn2C([2H])([2H])[C@]([2H])(N(C)C)C([2H])([2H])[2H])c1] |
| 152 | 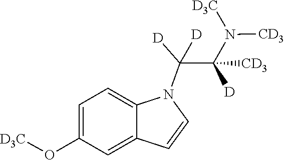 [SMILES: [2H]C([2H])([2H])Oc1ccc2c(ccn2C([2H])([2H])[C@]([2H])(N(C([2H])([2H])[2H])C([2H])([2H])[2H])C([2H])([2H])[2H])c1] |
| 153 | 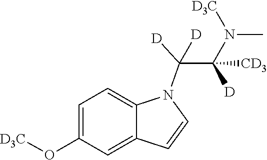 [SMILES: [2H]C([2H])([2H])Oc1ccc2c(ccn2C([2H])([2H])[C@]([2H])(N(C)C([2H])([2H])[2H])C([2H])([2H])[2H])c1] |
| 154 | 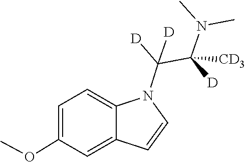 |
| 155 | 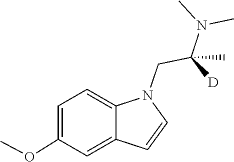 |
| 156 | 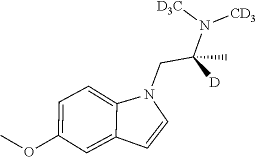 [SMILES: [2H]C([2H])([2H])N(C([2H])([2H])[2H])[C@]([2H])(C)Cn1ccc2cc(OC)ccc21] |
| 157 | 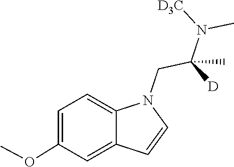 |
| 158 | 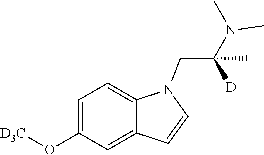 [SMILES: [2H]C([2H])([2H])Oc1ccc2c(ccn2C[C@@]([2H])(C)N(C)C)c1] |
| 159 | 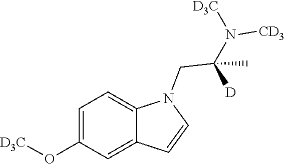 [SMILES: [2H]C([2H])([2H])Oc1ccc2c(ccn2C[C@@]([2H])(C)N(C([2H])([2H])[2H])C([2H])([2H])[2H])c1] |
| 160 | 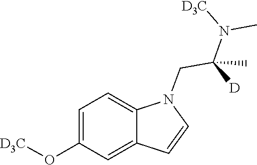 [SMILES: [2H]C([2H])([2H])Oc1ccc2c(ccn2C[C@@]([2H])(C)N(C)C([2H])([2H])[2H])c1] |
| 161 | 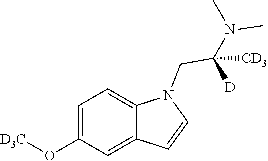 [SMILES: [2H]C([2H])([2H])Oc1ccc2c(ccn2C[C@]([2H])(N(C)C)C([2H])([2H])[2H])c1] |
| 162 | 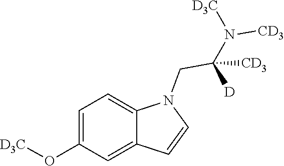 [SMILES: [2H]C([2H])([2H])Oc1ccc2c(ccn2C[C@]([2H])(N(C([2H])([2H])[2H])C([2H])([2H])[2H])C([2H])([2H])[2H])c1] |
| 163 | 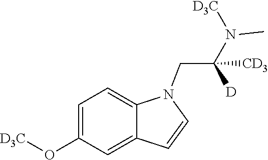 [SMILES: [2H]C([2H])([2H])Oc1ccc2c(ccn2C[C@]([2H])(N(C)C([2H])([2H])[2H])C([2H])([2H])[2H])c1] |
| 164 | 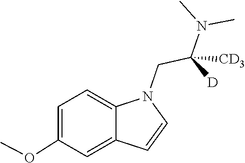 |
| 165 | 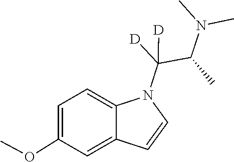 |
| 166 | 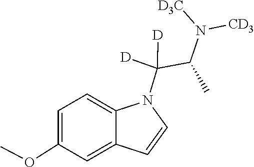 [SMILES: [2H]C([2H])([2H])N([C@H](C)C([2H])([2H])n1ccc2cc(OC)ccc21)C([2H])([2H])[2H]] |
| 167 | 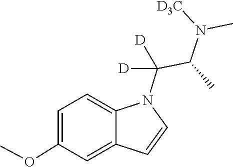 |
| 168 | 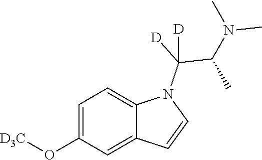 [SMILES: [2H]C([2H])([2H])Oc1ccc2c(ccn2C([2H])([2H])[C@@H](C)N(C)C)c1] |
| 169 | 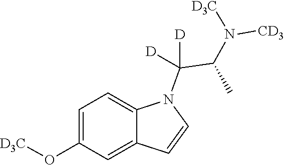 [SMILES: [2H]C([2H])([2H])Oc1ccc2c(ccn2C([2H])([2H])[C@@H](C)N(C([2H])([2H])[2H])C([2H])([2H])[2H])c1] |
| 170 | 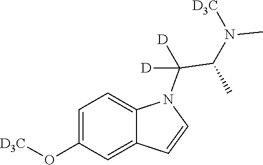 [SMILES: [2H]C([2H])([2H])Oc1ccc2c(ccn2C([2H])([2H])[C@@H](C)N(C)C([2H])([2H])[2H])c1] |

or a stereoisomer or enantiomer thereof.

In some embodiments, the compound of Formula I, II or III is isotopically enriched. Certain embodiments of such as isotopically enriched compounds have Formula I′:

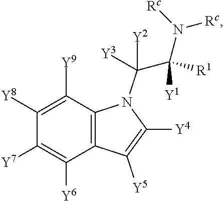

or
or an enantiomer or diastereomer thereof, wherein
R¹ is selected from C₁₋₆ alkyl, C₃₋₈ cycloalkyl, or C₄₋₁₄ alkyl-cycloalkyl;
Y¹, Y², Y³, Y⁴, Y⁵, Y⁶, Y⁷, Y⁸ and Y⁹ are each independently R^{b}, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{a}, —NO₂, —CN, —C(O)R^{b}, —C(O)OR^{b}, —OC(O)R^{b}, —OC(O)OR^{b}, —N(R^{yc}R^{yc}), —N(R^{b})C(O)R^{b}, —C(O)N(R^{yc}R^{yc}), —N(R^{b})C(O)OR^{b}, —OC(O)N(R^{c}R^{c}), —N(R^{b})C(O)N(R^{yc}R^{yc}), —C(O)C(O)N(R^{yc}R^{yc}), —S(O₂)R^{b}, —S(O)₂N(R^{yc}R^{yc}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{a} is C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{b} is, for each occurrence, independently hydrogen, deuterium, or C₁₋₆ alkyl;
R^{yc} is, for each occurrence, independently selected from hydrogen, deuterium, C₁₋₆ alkyl, C₃₋₈ cycloalkyl, and C₄₋₁₄ alkyl-cycloalkyl, or two R^{yc} together with the nitrogen to which they are attached form a C₂₋₁₂ heterocycloalkyl; and
R^{c} is, for each occurrence, selected from hydrogen, deuterium, C₁₋₆ alkyl, C₃₋₈ cycloalkyl, and C₄₋₁₄ alkyl-cycloalkyl, or two of R^{c} and R¹ together with the atoms to which they are attached to form a C₂₋₁₂ heterocycloalkyl;
alternatively, one of R^{c} and R¹ is combined with Y⁴ to form a C₅₋₁₂ heterocycloalkyl;
alternatively, Y⁴ and Y⁵ are combined with the atoms to which they are each attached to form a C₄₋₈ cycloalkyl, C₄₋₁₀ heterocycloalkyl, or C₆₋₁₂ aryl; alternatively, Y⁶ and R², R² and Y⁷, or Y⁷ and Yare combined with the atoms to which they are each attached to form a C₄₋₆ cycloalkyl, C₄₋₆ heterocycloalkyl, C₆₋₁₂ aryl, or C₅₋₁₀ heteroaryl.

Isotopically enriched compounds disclosed herein, such as those according to Formula I, can be enriched in any suitable isotope that improves a property of the molecule. For example, any site with a hydrogen atom can be enriched in deuterium or tritium by replacement of protium with these heavy isotopes. Similarly, a molecule with carbon at a particular position can be enriched in ¹⁴C.

Additional embodiments of isotopically enriched compounds disclosed herein, such as those of Formula I, have an ether moiety at the Y⁷ position.

Certain embodiments of compounds according to Formula I wherein Y⁷ forms an ether group have Formula II′

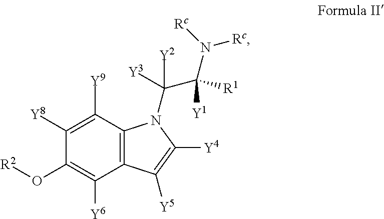

or
an enantiomer or diastereomer thereof, wherein
R² is selected from C₁₋₆ alkyl, C₃₋₈ cycloalkyl, C₃₋₁₄ alkyl-cycloalkyl, C₁₋₆ haloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl and C₄₋₁₆ alkyl-heteroaryl.

Further embodiments of isotopically enriched compounds, including compounds of Formulas I and II, or an enantiomer or diastereomer thereof, are represented by Formula II″, wherein R¹ is selected from C₁₋₆ alkyl, C₃₋₈ cycloalkyl, or C₄₋₁₄ alkyl-cycloalkyl;
Y¹, Y Y³, Y⁴, Y⁵, Y⁶, Y⁸ and Y⁹ are each independently selected from hydrogen, deuterium, halogen and C₁₋₆ alkyl,
R² is selected from C₁₋₆ alkyl, C₁₋₆ haloalkyl and C₃₋₈ cycloalkyl; and
R^{c} is, for each occurrence, selected from C₁₋₆ alkyl, C₃₋₈ cycloalkyl, or C₄₋₁₄ alkyl-cycloalkyl, or two of R^{c} and R¹ together with the atoms to which they are attached to form a C₂₋₁₂ heterocycloalkyl.

Still further disclosed embodiments of compounds of Formulas I and II, or an enantiomer or diastereomer thereof, include isotopically enriched compounds are represented by Formula II″′, wherein
R¹ is selected from C₁₋₆ alkyl;
Y¹, Y Y³, Y⁴, Y⁵, Y⁶, Y⁸ and Y⁹ are each independently selected from hydrogen, deuterium, halogen and C₁₋₆ alkyl,
R² is selected from C₁₋₆ alkyl, C₁₋₆ haloalkyl and C₃₋₈ cycloalkyl; and
R^{c} is, for each occurrence, selected from hydrogen, deuterium, C₁₋₆ alkyl, or two R^{c} together with the nitrogen to which they are attached to form a C₂₋₁₂ heterocycloalkyl.

In embodiments of the compounds disclosed herein, including those represented by Formulas I and II, the compounds are enriched in deuterium, tritium, carbon-14 or a combination thereof.

In particular embodiments of isotopically enriched compounds of Formulas I and II, or an enantiomer or diastereomer thereof, the isotopically enriched compounds disclosed herein have at least one of Y¹, Y², Y⁴, Y⁵, Y⁶, Y⁷, Y⁸, Y⁹, R¹, R² and R^{c} enriched in deuterium, tritium, carbon-14, or a combination thereof. For example, in certain embodiments at least one of Y¹, Y², Y³, Y⁴, Y⁵, Y⁶, Y⁷, Y⁸, Y⁹, R¹, R² and R^{c} is enriched in deuterium, such as is the case when at least one of Y¹, Y², Y³, Y⁴, Y⁵, Y⁶, Y⁷, Y⁸ and Y⁹ is enriched in deuterium. Additional compounds disclosed herein have at least one of R¹, R² and R^{c} isotopically enriched in deuterium, such as at least one R^{c} is enriched in deuterium.

In particular examples of the embodiments described above, R¹, R² and R^{c} each are methyl wherein the methyl groups optionally are isotopically enriched in deuterium. Examples of such compounds include those wherein R¹ and R² are independently selected from CH₃, CH₂D, CHD₂ and CD₃.

Examples of the compounds described above are represented by Formulas IIA-IIG, or enantiomers or diastereomers thereof:

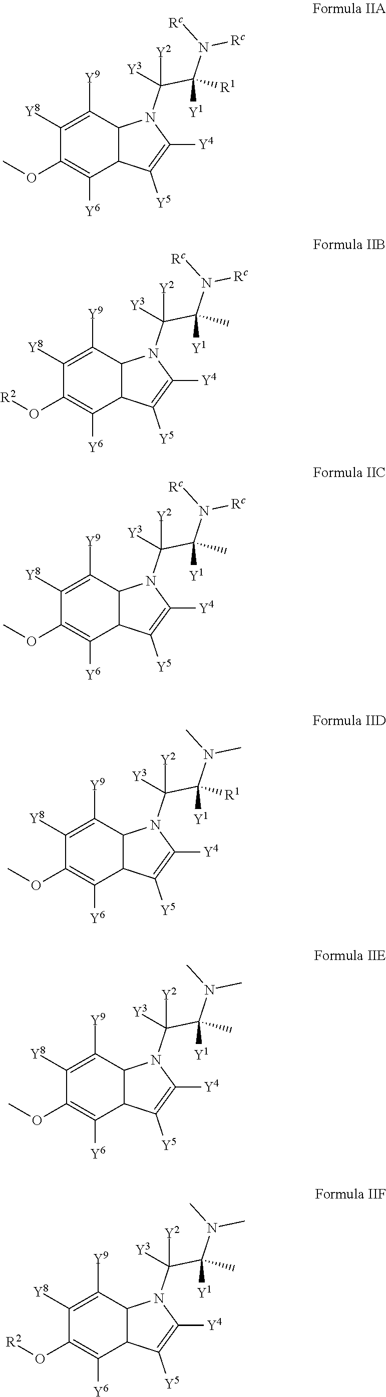

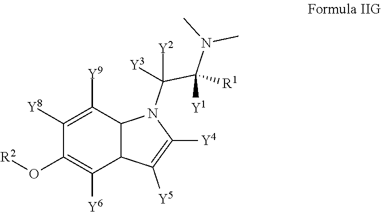

With reference to Formulas IIA-IIG, R¹, R² and R^{c} each are independently selected from CH₃, CH₂D, CHD₂ and CD₃ and Y¹, Y², Y³, Y⁴, Y⁵, Y⁶, Y⁸ and Y⁹ each are independently selected from H, D. In a further embodiment of Formulas IIA-IIG, at least one of R¹, R², R^{c}, Y¹, Y², Y³, Y⁴, Y⁵, Y⁶, Y⁸ and Y⁹ are enriched for deuterium.

Additional examples of the compounds described above are represented by Formulas IIIA-IIIG, or enantiomers or diastereomers thereof:

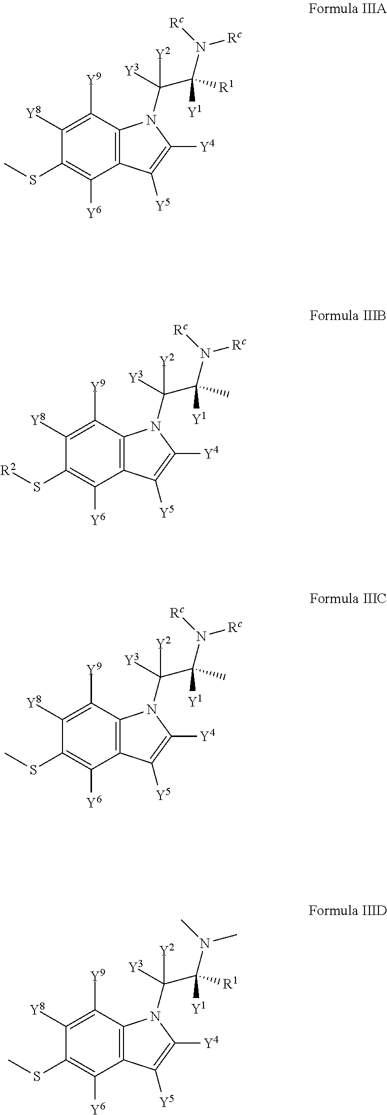

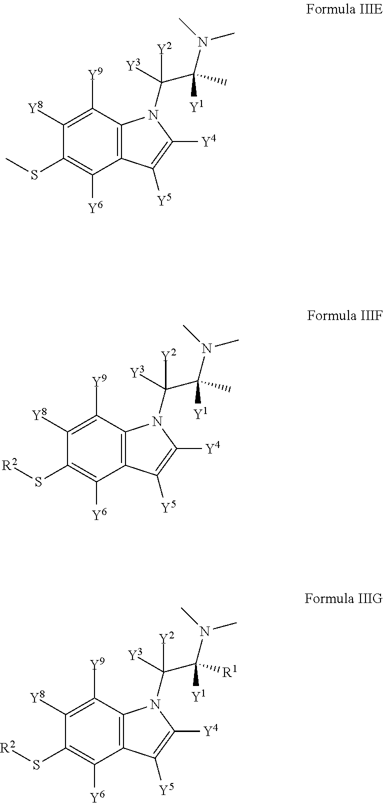

With reference to Formulas IIIA-IIIG, R¹, R² and R^{c} each are independently selected from CH₃, CH₂D, CHD₂ and CD₃ and Y¹, Y², Y³, Y⁴, Y⁵, Y⁶, Y⁸ and Y⁹ each are independently selected from H, D; or R² and Y⁶ together with the atoms to which they are attached form a C₄₋₆ heterocycloalkyl or C₄₋₁₀ heteroaryl. In a further embodiment of Formulas IIA-IIG, at least one of R¹, R², R^{c}, Y¹, Y², Y³, Y⁴, Y⁵, Y⁶, Y⁸ and Y⁹ are enriched for deuterium.

With continued reference to Formulas I, II, IIA-IIG, III, and IIIA-IIIG, as is understood by those of ordinary skill in the art, when Y² and Y³ are different, the Formulas contain two stereocenters. In such compounds all diastereomers, i.e., the (R,R), (S,S), (R,S) and (S,R) diastereomers are specifically intended. The chiral compounds disclosed herein can be synthesized using enantioselective techniques as is known to those of ordinary skill in the art. Moreover, diastereomeric and enantiomeric products can be separated by chromatography, fractional crystallization and other methods known to those of ordinary skill in the art.

In other examples of the embodiments of Formulas I, II and III, the groups Y¹, Y², Y³, Y⁴, Y⁵, Y⁶, Y⁸ and Y⁹ each independently are selected from protium and deuterium.

More particular embodiments of the disclosed isotopically enriched compounds of Formulas I, II and IIA-IIG have the formulas illustrated below:

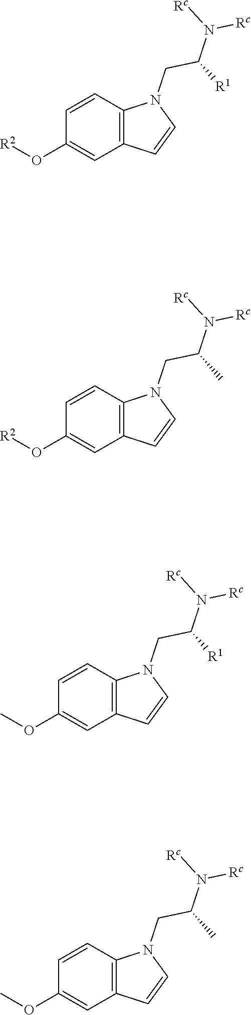

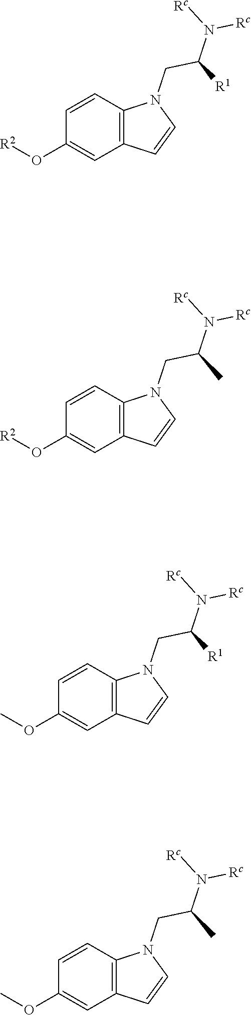

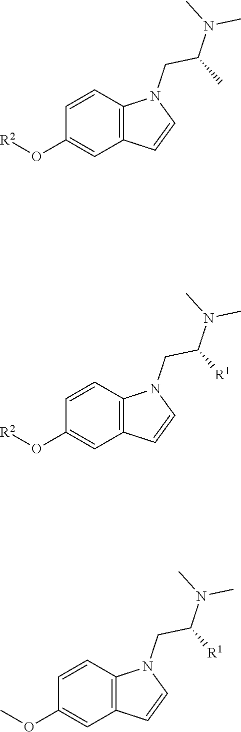

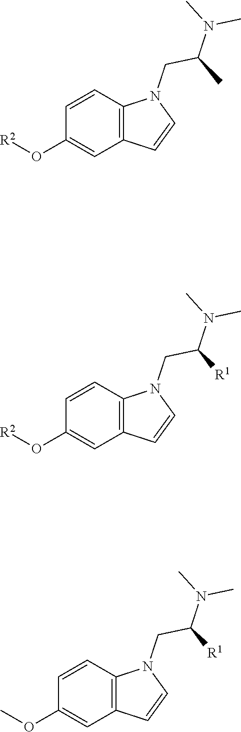

In still more particular embodiments of the disclosed isotopically enriched compounds of Formulas I, II, IIA-IIG, and III, the compounds are isotopically enriched as illustrated below:

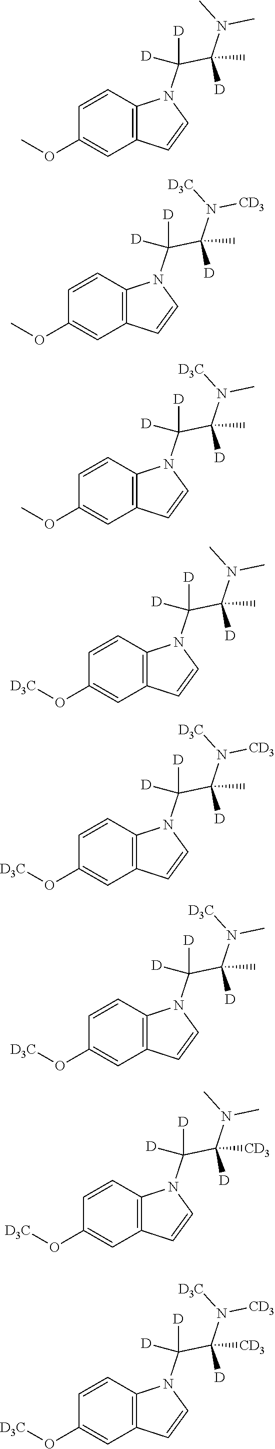

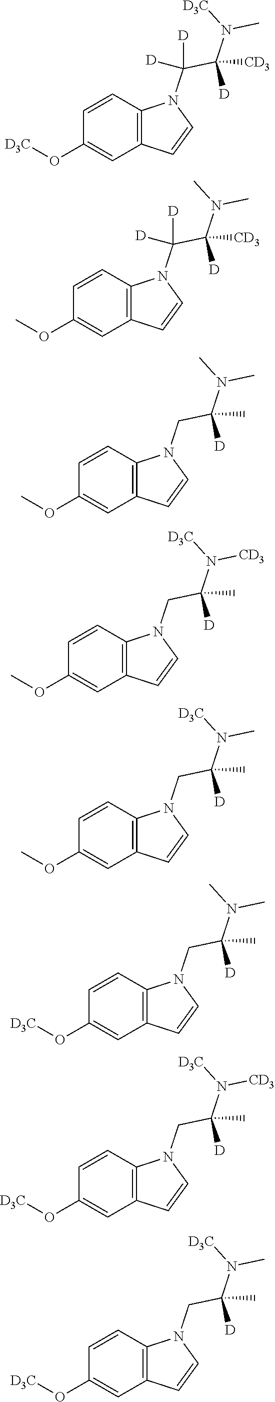

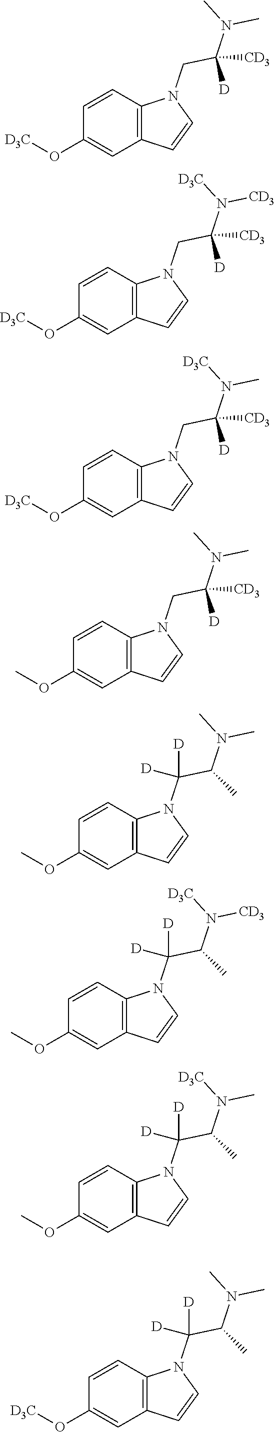

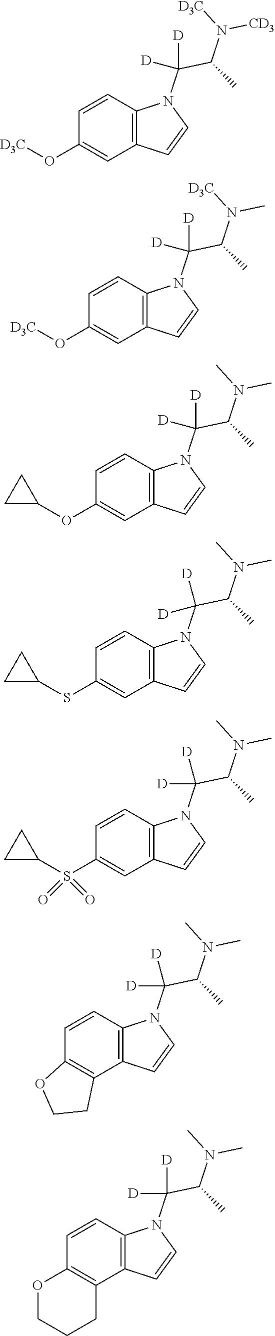

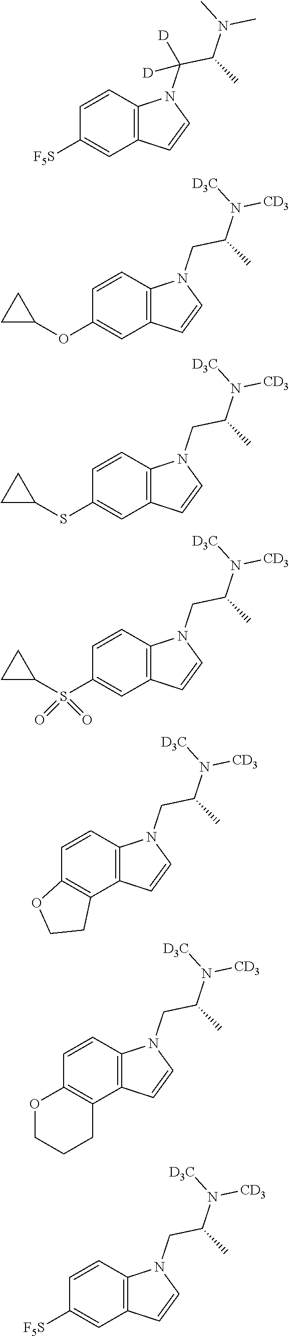

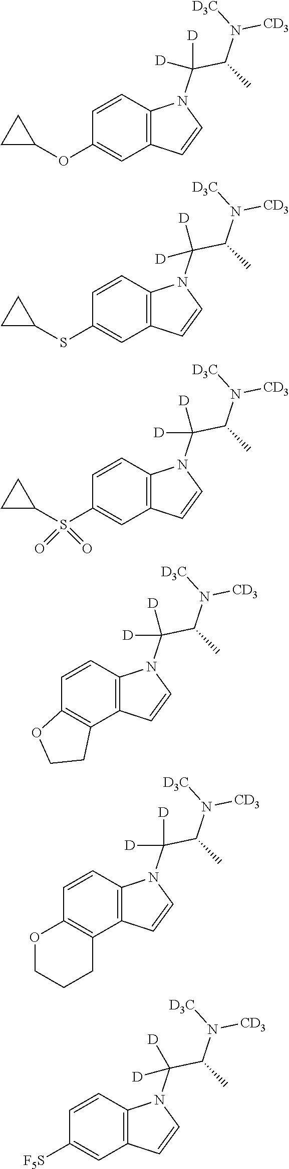

or
an enantiomer or diastereomer thereof.

In certain embodiments of Formulas I, II, IIA-IIG, and III, R¹ and Y¹ both are methyl. Examples of such compounds have R¹ and Y¹ independently selected from CH₃, CH₂D, CHD₂ and CD₃. By way of example, embodiments of compounds of Formulas I, II, IIA-IIG and III, wherein R¹ and Y¹ both are methyl include

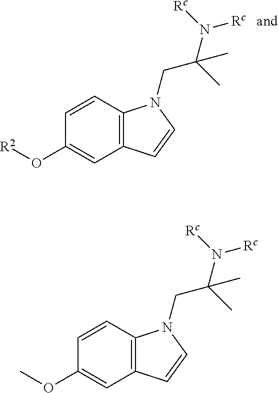

In further examples of compounds according to Formulas I, II, IIA-IIG, and/or III, the compounds are isotopically enriched as illustrated below:

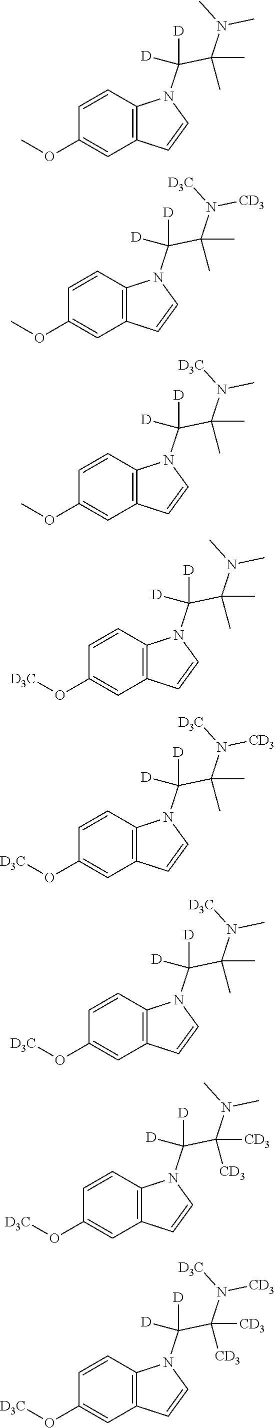

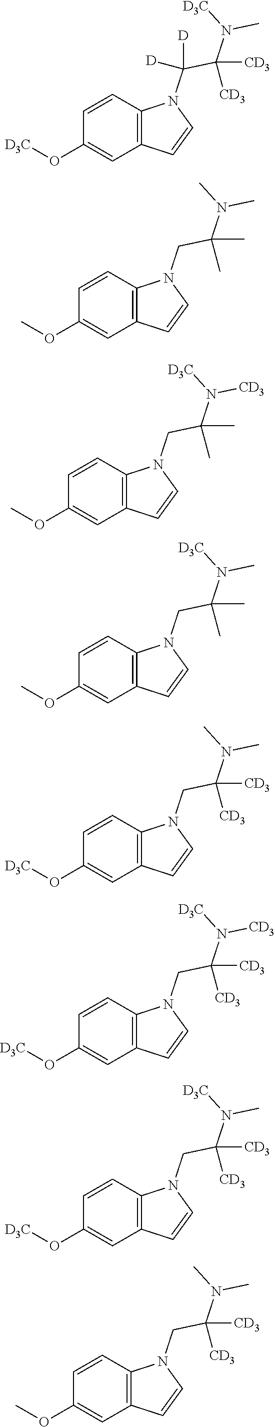

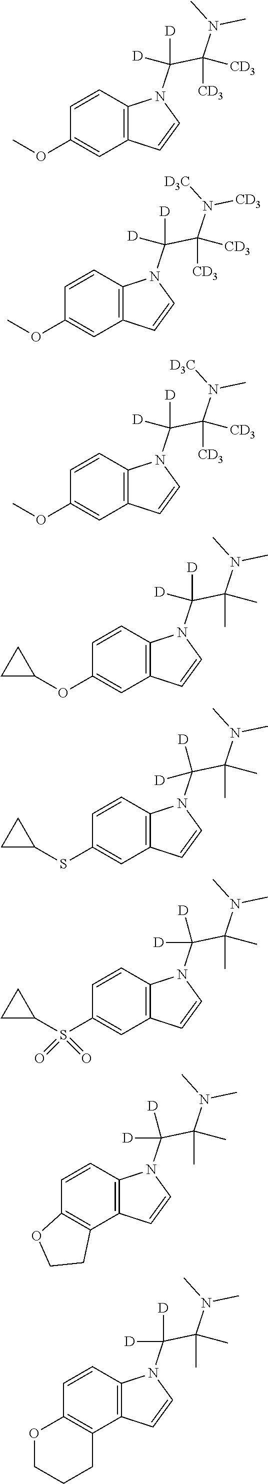

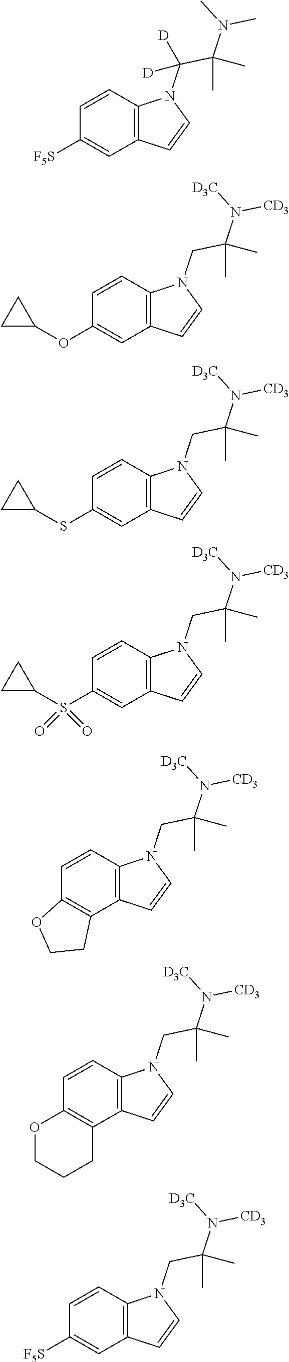

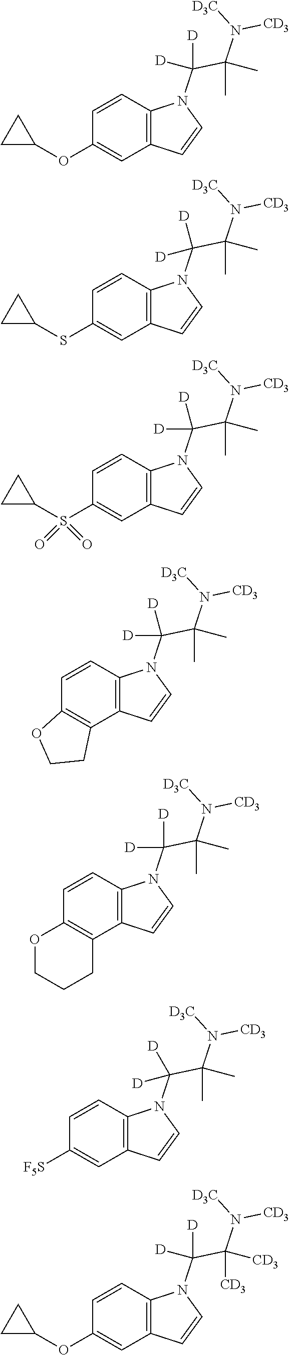

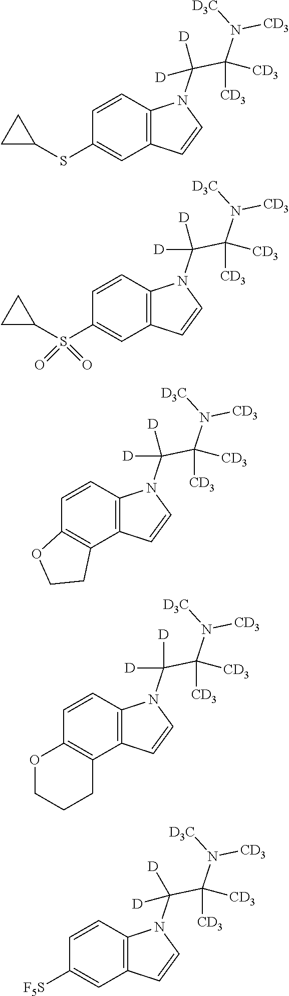

or
an enantiomer or diastereomer thereof.

In other particular examples, the compounds of Formulas I and II have the formula

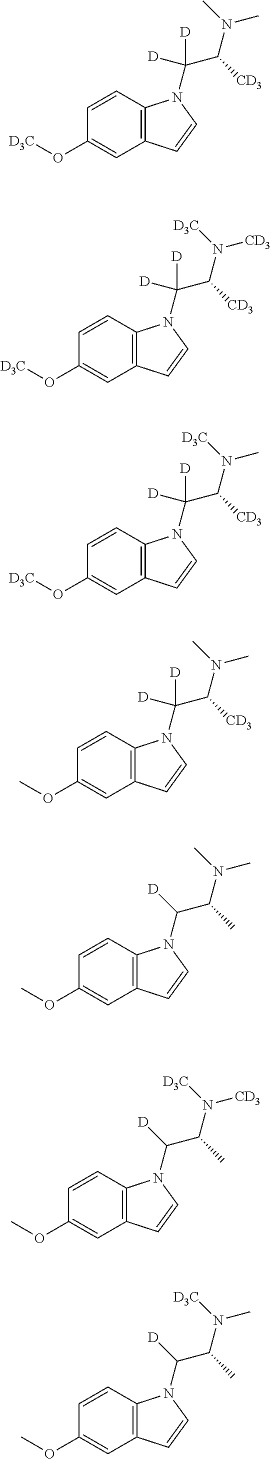

any diastereomer of any of the above

As noted above, in compounds having two or more stereocenters, such as those above, all diastereomers are specifically envisioned. Thus, by way of example, by each of the structures illustrated, the (R,R), (S,S), (R,S) and (S,R) diastereomers are intended.

By way of further illustration, the structure

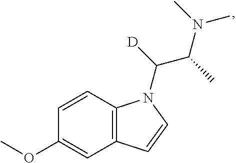

thus encompasses the specific diastereomers:

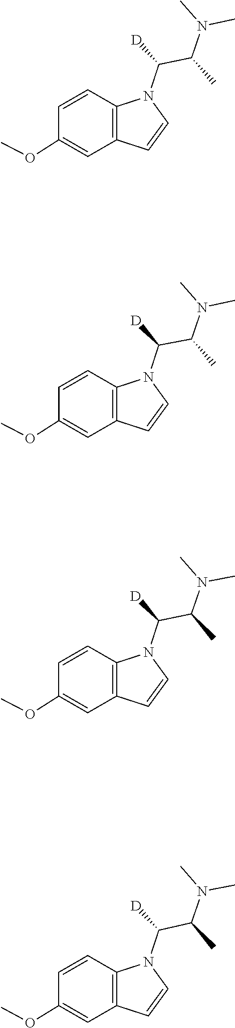

In still further embodiments of the disclosed isotopically enriched compounds of Formulas I, II and IIA-IIG, the compounds are isotopically enriched as illustrated below:

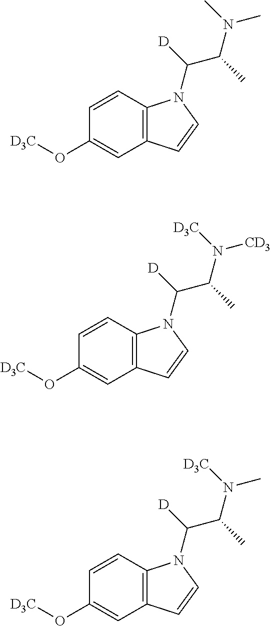 [SMILES: [2H]C([C@@H](C)N(C)C)n1ccc2cc(OC([2H])([2H])[2H])ccc21 ; [2H]C([C@@H](C)N(C([2H])([2H])[2H])C([2H])([2H])[2H])n1ccc2cc(OC([2H])([2H])[2H])ccc21 ; [2H]C([C@@H](C)N(C)C([2H])([2H])[2H])n1ccc2cc(OC([2H])([2H])[2H])ccc21]

any diastereomer of any of the above

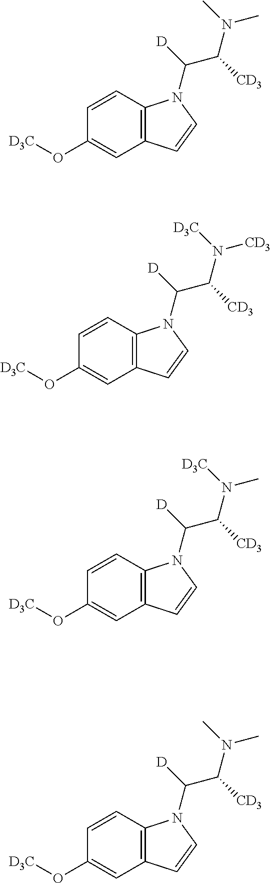 [SMILES: [2H]C([C@H](N(C)C)C([2H])([2H])[2H])n1ccc2cc(OC([2H])([2H])[2H])ccc21 ; [2H]C([C@H](N(C([2H])([2H])[2H])C([2H])([2H])[2H])C([2H])([2H])[2H])n1ccc2cc(OC([2H])([2H])[2H])ccc21 ; [2H]C([C@H](N(C)C([2H])([2H])[2H])C([2H])([2H])[2H])n1ccc2cc(OC([2H])([2H])[2H])ccc21 ; [2H]C([C@H](N(C)C)C([2H])([2H])[2H])n1ccc2cc(OC([2H])([2H])[2H])ccc21]

any diastereomer of any of the above

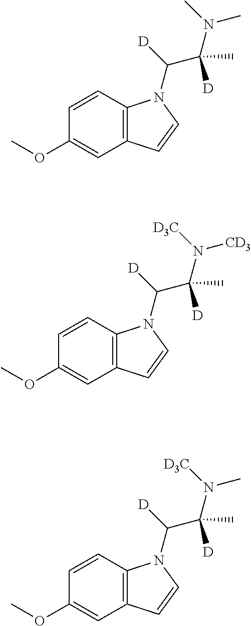

any diastereomer of any of the above

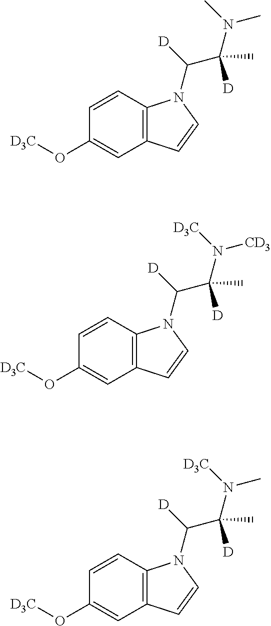 [SMILES: [2H]C(n1ccc2cc(OC([2H])([2H])[2H])ccc21)[C@@]([2H])(C)N(C)C ; [2H]C(n1ccc2cc(OC([2H])([2H])[2H])ccc21)[C@@]([2H])(C)N(C([2H])([2H])[2H])C([2H])([2H])[2H] ; [2H]C(n1ccc2cc(OC([2H])([2H])[2H])ccc21)[C@@]([2H])(C)N(C)C([2H])([2H])[2H]]

any diastereomer of any of the above

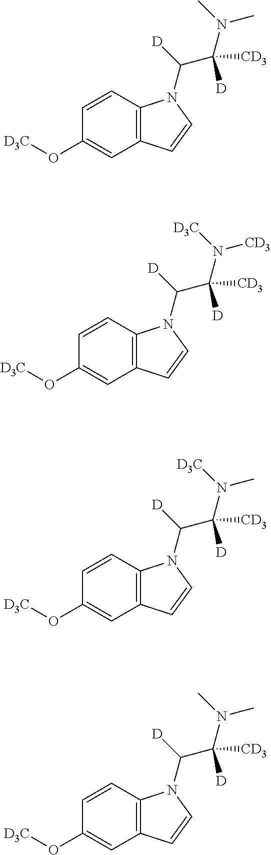 [SMILES: [2H]C(n1ccc2cc(OC([2H])([2H])[2H])ccc21)[C@]([2H])(N(C)C)C([2H])([2H])[2H] ; [2H]C(n1ccc2cc(OC([2H])([2H])[2H])ccc21)[C@]([2H])(N(C([2H])([2H])[2H])C([2H])([2H])[2H])C([2H])([2H])[2H] ; [2H]C(n1ccc2cc(OC([2H])([2H])[2H])ccc21)[C@]([2H])(N(C)C([2H])([2H])[2H])C([2H])([2H])[2H] ; [2H]C(n1ccc2cc(OC([2H])([2H])[2H])ccc21)[C@]([2H])(N(C)C)C([2H])([2H])[2H]]

any diasteromers of any of the above

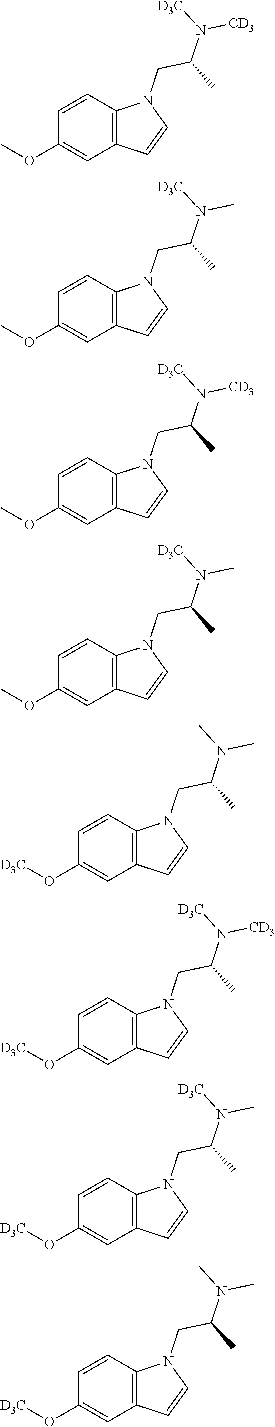

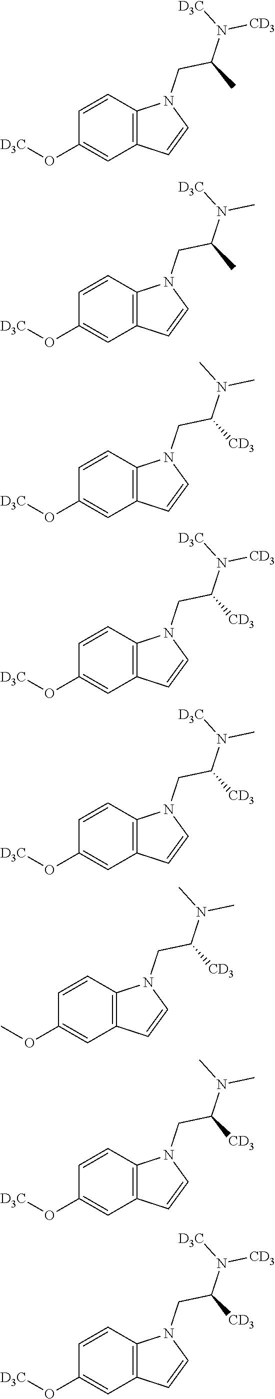

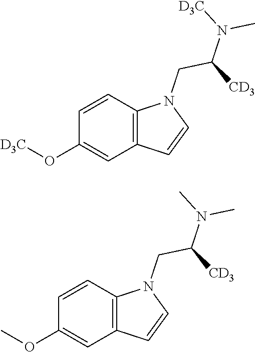

The compounds of the present invention can also be in salt forms, such as acid or base salts of the disclosed compounds. Illustrative examples of pharmaceutically acceptable acid salts are mineral acid (hydrochloric acid, hydrobromic acid, phosphoric acid, and the like) salts, organic acid (fumaric acid, acetic acid, propionic acid, glutamic acid, citric acid, tartaric acid and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

In addition, all physical forms of the compounds of Formulas I, II, IIA and JIB are intended herein, including the compounds of Formulas I, II, IIA and IIB, in the form of solvates, such as hydrates. Moreover, non-crystalline and crystalline forms of the compounds of Formulas I, II, and IIA-IIG, including amorphous forms, isomorphs and polymorphs are within the scope of the present invention.

### Pharmaceutical Compositions and Formulations

In some embodiments, the present invention provides a pharmaceutical composition comprising a compound of the present invention, such as a composition comprising a compound of Formulas I, II, or IIA-IIG, illustrated above, and a pharmaceutically acceptable excipient. Such compositions are suitable for administration to a subject, such as a human subject.

The presently disclosed pharmaceutical compositions can be prepared in a wide variety of oral, parenteral and topical dosage forms. Oral preparations include tablets, pills, powder, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compounds of the present invention.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% to 70% or 10% to 70% of the compounds of the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethylcellulose, or sodium carboxymethylcellulose; and gums including arabic and tragacanth; as well as proteins including, but not limited to, gelatin and collagen.

If desired, disintegrating or solubilizing agents may be added, such as the cross-linked polyvinyl pyrrolidone, agar, alginic acid, or a salt thereof, such as sodium alginate.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In some embodiments, the pharmaceutical compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the formulations of the compositions of the present invention can be delivered by the use of liposomes which fuse with the cellular membrane or are endocytosed, for example, by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J Microencapsul. 13:293-306, 1996; Chonn, Curr. Opin. Biotechnol. 6:698-708, 1995; Ostro, Am. J Hosp. Pharm. 46:1576-1587, 1989).

### V. Administration

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, and the like as is known to those of ordinary skill in the art. Suitable dosage ranges for the compounds disclosed herein include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds disclosed herein can be administered at any suitable frequency, interval and duration. For example, the compounds can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with a second active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, such as by preparing a single pharmaceutical composition including both the compound of the present invention and a second active agent. In other embodiments, the compound of the present invention and the second active agent can be formulated separately.

The disclosed compounds and the second active agent can be present in the compositions of the present invention in any suitable weight ratio, such as from about 1:100 to about 100: 1 (w/w), or about 1:50 to about 50: 1, or about 1:25 to about 25: 1, or about 1:10 to about 10:1, or about 1:5 to about 5:1 (w/w). The compound of the present invention and the second active agent can be present in any suitable weight ratio, such as about 1: 100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods disclosed herein.

### Methods of Treatment

The compounds of the present invention can be used for increasing neuronal plasticity. The compounds of the present invention can also be used to treat any brain disease. The compounds of the present invention can also be used for increasing at least one of translation, transcription or secretion of neurotrophic factors.

In some embodiments, a compound of the present invention is used to treat neurological diseases. In some embodiments, the compounds have, for example, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound of the present invention is used for increasing neuronal plasticity. In some embodiments, the compounds described herein are used for treating a brain disorder. In some embodiments, the compounds described herein are used for increasing at least one of translation, transcription, or secretion of neurotrophic factors.

In some embodiments, the compounds of the present invention have activity as 5-HT_{2A} modulators. In some embodiments, the compounds of the present invention have activity as 5-HT_{2A} modulators. In some embodiments, the compounds of the present invention elicit a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity (Ly et al., 2018). 5-HT_{2A} antagonists abrogate the neuritogenesis and spinogenesis effects of hallucinogenic compounds with 5-HT_{2A} agonist activity, for example., DMT, LSD, and DOI. In some embodiments, the compounds of the present invention are 5-HT_{2A} modulators and promote neural plasticity (e.g., cortical structural plasticity). In some embodiments, the compounds of the present invention are selective 5-HT_{2A} modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, non-hallucinogenic 5-HT_{2A} modulators (e.g., 5-HT_{2A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-HT_{2A} agonists) are used for increasing at least one of translation, transcription, or secretion of neurotrophic factors.

### Methods for Increasing Neuronal Plasticity

Neuronal plasticity refers to the ability of the brain to change structure and/or function throughout a subject's life. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present invention provides methods for increasing neuronal plasticity, comprising contacting a neuronal cell with any of the compounds of the present invention. In some embodiments, increasing neuronal plasticity improves a brain disorder described herein.

In some embodiments, a compound of the present invention is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neuronal plasticity of any compound of the present invention is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present invention is a mouse head-twitch response (HTR) assay.

In some embodiments, the present invention provides a method for increasing neuronal plasticity, comprising contacting a neuronal cell with a compound of Formula I, II, IIA-IIG, III or IIIA-IIIG.

### Methods of Treating a Brain Disorder

In some embodiments, the present invention provides a method of treating a disease, including administering to a subject in need thereof, a therapeutically effective amount of a compound of the present invention. In some embodiments, the present invention provides a method of treating a brain disorder, including administering to a subject in need thereof, a therapeutically effective amount of a compound disclosed herein, such as a compound of Formula I, IA, IA-i, IA-ii, IA-iii, IA-iv, IA-v, IB, II, II′, Ix, IIA-IIG, III, III′, IIIA-IIIG, IV, V, VI, VII, VIII, IX, X, XI, XII, XIII XIV, or XV or any one of the compounds described in Table 1 or a representative compound of the application, including but not limited to Compound 1, Compound 2, Compound 3, Compound 4, Compound 5, Compound 6, Compound 7, Compound 151, Compound 8, Compound 152, Compound 91, Compound 92, and Compound 93.

In some embodiments, the present invention provides a method of treating a brain disorder with combination therapy, including administering to a subject in need thereof, a therapeutically effective amount of a compound of the present invention and at least one additional therapeutic agent.

In some embodiments, serotonin receptor modulators, such as modulators of serotonin receptor 2A (5-HT_{2A} modulators, e.g., 5-HT_{2A} agonists), are used to treat a brain disorder. The presently disclosed compounds, e.g., those of Formula I, IA, IA-i, IA-ii, IA-iii, IA-iv, IA-v, IB, II, II′, IIx, IIA-IIG, III, III′, IIIA-IIIG, IV, V, VI, VII, VIII, IX, X, XI, XII, XIII XIV, or XV can function as 5-HT_{2A} agonists alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present invention to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, AMDA, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, risperidone, paliperidone, asenapine, amisulpride, aripiprazole, brexpiprazole, lurasidone, ziprasidone, lumateperone, perospirone, mosapramine, adatanserin, AMDA (9-Aminomethyl-9,10-dihydroanthracene), cinanserin, fananserin, iferanserin, methiothepin, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, and an extended-release form of aripiprazole including Abilify Maintena, or a pharmaceutically acceptable salt, solvate, metabolite, deuterated analog, derivative, prodrug, or combinations thereof. In some embodiments, the serotonin receptor modulator used as a second therapeutic is pimavanserin or a pharmaceutically acceptable salt, solvate, metabolite, derivative, or prodrug thereof.

In some embodiments, the serotonin receptor modulator is administered prior to a compound disclosed herein, including those described in Table 1, such as about three hours prior or from about one to about three hours prior to administration of a compound disclosed herein, including those described in Table 1 or according to Formula I, IA, IA-i, IA-ii, IA-iii, IA-iv, IA-v, IB, II, II′, Ix, IIA-IIG, III, III′, IIIA-IIIG, IV, V, VI, VII, VIII, IX, X, XI, XII, XIII XIV, or XV or a pharmaceutically acceptable salt thereof. In some embodiments, the serotonin receptor modulator is administered at most about one hour prior to the presently disclosed compound, including those described in Table 1. Thus, in some embodiments of combination therapy with the presently disclosed compounds, including those described in Table 1, the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, the compound of the present invention, including those described in Table 1, is provided at a dose of from about 10 mg to about 100 mg, or from about 20 to about 200 mg, or from about 15 to about 300 mg, and the serotonin receptor modulator is provided at a dose of about 1 mg to about 100 mg.

In some embodiments, the brain disorders that can be treated as disclosed herein comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, a compound of the present disclosure is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, schizophrenia, and addiction (e.g., substance abuse disorder). In some embodiments, brain disorders include, for example, migraines, addiction (e.g., substance use disorder), depression, and anxiety.

In some embodiments, the brain disorder is a neurodegenerative disorder, Alzheimer's, Parkinson's disease, psychological disorder, depression, addiction, anxiety, post-traumatic stress disorder, treatment resistant depression, postpartum depression, premenstrual dysphoric disorder, seasonal affective disorder, suicidal ideation, major depressive disorder, bipolar disorder, schizophrenia, stroke, traumatic brain injury, or substance use disorder.

In some embodiments, the brain disorder is a neurodegenerative disorder, Alzheimer's, or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, persistent depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent that is lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In certain embodiments of the method for treating a brain disorder disclosed herein with a compound according to any one of the Formulae disclosed herein Formula I, IA, IA-i, IA-ii, IA-iii, IA-iv, IA-v, IB, II, II′, Ix, IIA-IIG, III, III′, IIIA-IIIG, IV, V, VI, VII, VIII, IX, X, XI, XII, XIII XIV, or XV, a second therapeutic agent that is an empathogenic agent is administered. Examples of suitable empathogenic agents for use in combination with a compound according to Formula I, IA, IA-i, IA-ii, IA-iii, IA-iv, IA-v, IB, II, II′, Ix, IIA-IIG, III, III′, IIIA-IIIG, IV, V, VI, VII, VIII, IX, X, XI, XII, XIII XIV, or XV are selected from the phenethylamines, such as 3,4-methylene-dioxymethamphetamine (MDMA) and analogs thereof. Other suitable empathogenic agents for use in combination with the presently disclosed compounds include, without limitation,
N-Allyl-3,4-methylenedioxy-amphetamine (MDAL)
N-Butyl-3,4-methylenedioxyamphetamine (MDBU)
N-Benzyl-3,4-methylenedioxyamphetamine (MDBZ)
N-Cyclopropylmethyl-3,4-methylenedioxyamphetamine (MDCPM)
N,N-Dimethyl-3,4-methylenedioxyamphetamine (MDDM)
N-Ethyl-3,4-methylenedioxyamphetamine (MDE; MDEA)
N-(2-Hydroxyethyl)-3,4-methylenedioxy amphetamine (MDHOET)
N-Isopropyl-3,4-methylenedioxyamphetamine (MDIP)
N-Methyl-3,4-ethylenedioxyamphetamine (MDMC)
N-Methoxy-3,4-methylenedioxyamphetamine (MDMEO)
N-(2-Methoxyethyl)-3,4-methylenedioxyamphetamine (MDMEOET)
alpha,alpha,N-Trimethyl-3,4-methylenedioxyphenethylamine (MDMP;
3,4-Methylenedioxy-N-methylphentermine)
N-Hydroxy-3,4-methylenedioxyamphetamine (MDOH)
3,4-Methylenedioxyphenethylamine (MDPEA)
alpha,alpha-Dimethyl-3,4-methylenedioxyphenethylamine (MDPH; 3,4-methylenedioxyphentermine)
N-Propargyl-3,4-methylenedioxyamphetamine (MDPL)
N-Propyl-3,4-methylenedioxyamphetamine (MDPR), and the like.

In some embodiments, the compounds of the present invention are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors

Neurotrophic factors refers to a family of soluble peptides or proteins which support the survival, growth, and differentiation of developing and mature neurons. Increasing at least one of translation, transcription, or secretion of neurotrophic factors can be useful for, but not limited to, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increasing neuronal plasticity. In some embodiments, increasing at least one of translation, transcription, or secretion of neurotrophic factors can promoting neuronal growth, promoting neuritogenesis, promoting synaptogenesis, promoting dendritogenesis, increasing dendritic arbor complexity, and/or increasing dendritic spine density.

In some embodiments, 5-HT_{2A} modulators (e.g., 5-HT_{2A} agonists) are used to increase at least one of translation, transcription, or secretion of neurotrophic factors. In some embodiments, a compound of the present invention is used to increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

In some embodiments, the present invention provides a method for increasing at least one of translation, transcription or secretion of neurotrophic factors, comprising contacting a neuronal cell with a compound disclosed herein, such as a compound of Formula I, II, IIA-IIG, III or IIIA-IIIG.

### EXAMPLES

### NMR Methodology

NMR analyses were conducted on a 400 MHz NMR spectrometer using deuterated chloroform, deuterated methanol or deuterated dimethyl sulfoxide as solvent. The shift (d) of each signal was measured in parts per million (ppm) relative to the residual solvent peak, and the multiplicity reported together with the associated coupling constant (J), where applicable.

### Agilent LC-MS Analysis Methodology

Instrument: Agilent 1260 infinity HPLC with Agilent 6130 single quadrupole mass spec.
Column: Phenomenex Kinetex XB-C₁₈, 50×4.6 mm, 2.6 μm
Elution profile: See table below

| | % AQUEOUS (A) | % ORGANIC (B) |
|---|---|---|
| TIME | (0.1% FORMIC | (100% |
| (MINUTES) | ACID IN WATER) | ACETONITRILE) |
| 0 | 95 | 5 |
| 1.37 | 2 | 98 |
| 1.60 | 2 | 98 |
| 1.83 | 95 | 5 |
| 2.25 | 95 | 5 |

Flow rate: 2 mL/min
Detector wavelength: 225±50 nm bandwidth
Column temperature: 40° C.
Injection volume: 1 μl
Mass spec parameters: Scanning in ES+/−& APCI over 70-1000m/z
Needle wash: MeOH wash in vial 4, autosampler set up to do 5 needle washes (to wash the outside of the needle prior to injecting the sample).
Sample preparation: 0.5-1.0 mg/ml in either acetonitrile or DMSO depending on the nature of the sample in terms of solubility.

### Waters Alliance LCMS Method Details

Acidic method
Instrument: Waters 2795 Alliance HPLC system equipped with a 2996 PDA detector and
Micromass ZQ mass spectrometer detector.
Column: Gemini C₁₈, 5 μm, 110 Å, 50×4.6 mm ID
Mobile phase A: 0.1% Formic acid in water
Mobile phase B: 100% acetonitrile
Gradient program (overall run time per injection is 8 minutes):

| | MOBILE | MOBILE |
|---|---|---|
| TIME | PHASE A | PHASE B |
| 0.0 | 95 | 5 |
| 0.6 | 95 | 5 |
| 6.50 | 2 | 98 |
| 7.50 | 2 | 98 |
| 7.60 | 95 | 5 |
| 8.00 | 95 | 5 |

Flow rate: 1 ml/min
Injection volume: 10 μl
Column oven temperature: 40° C.
Detector: PDA UV at 190-400 nm, also fixed λ at 225 nm
Mass spec parameters: MS scan in ES+, ES−, ranging from M/Z 100-1000
Purge solvent involved in injection

### Exemplary Compounds Disclosed Herein are Prepared According to General Scheme 1:

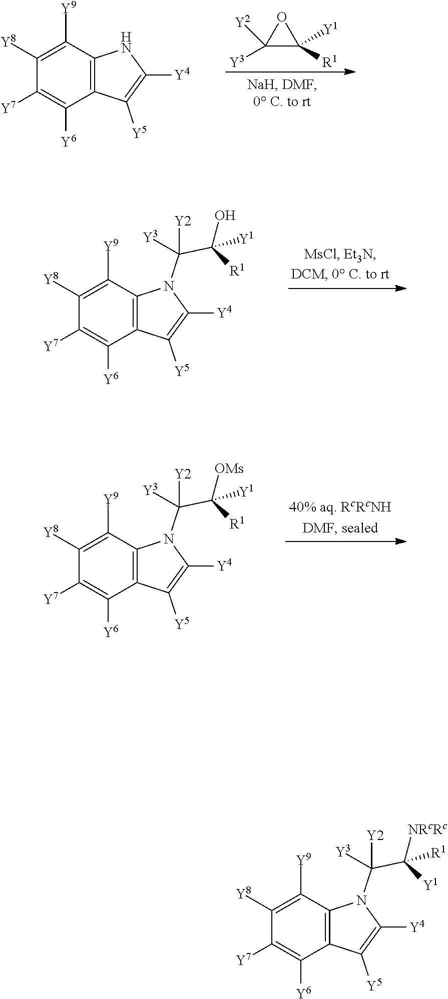

As is known to those of ordinary skill in the art, additional methods may be adapted to synthesize the compounds disclosed herein. See for example, Adams et al. Tetrahedron Lett. 2002 43, 7581-7583. For example, isotopically enriched building blocks can be substituted for non-enriched building blocks, including those described by Adams et al. to prepare isotopically enriched indole derivatives disclosed herein. Scheme 2 illustrates useful methods and materials for synthesizing presently disclosed compound.

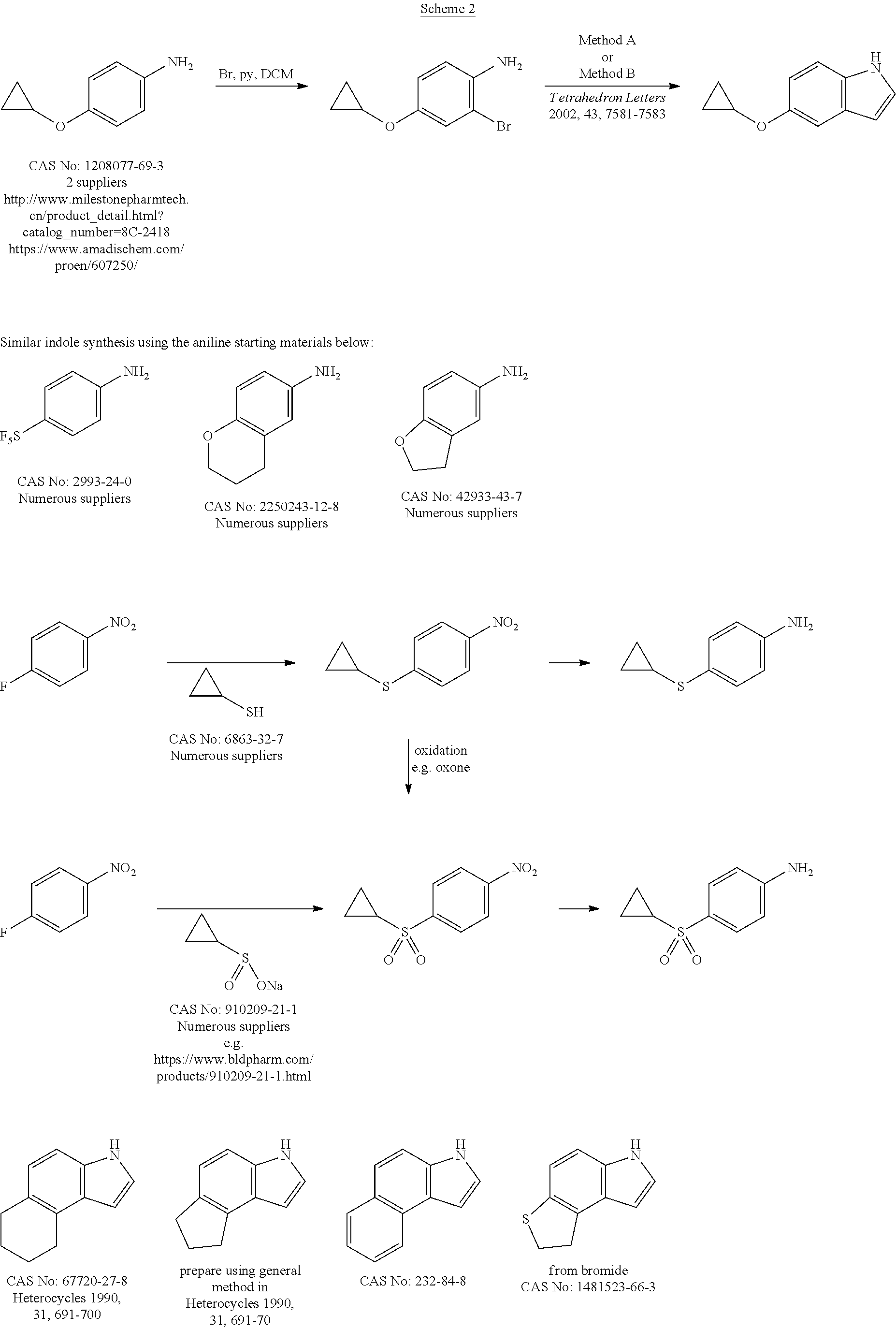

Exemplary starting materials that can be used to make the presently disclosed compounds include:

| | |
|---|---|
| 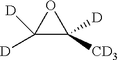 | CAS No: 1911606-71-7 Ref: Stavros et al, Angewandte Chemie, International Edition (2016), 55(21), 6225-6228 |
| 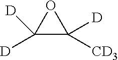 | CAS No: 202468-69-7 Supplier: https://shop.isotope.com/productdetails.aspx?itemno=DLM-1067-PK |
| 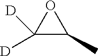 | CAS No: 1911615-27-4 Ref: Stavros et al, Angewandte Chemie, International Edition (2016), 55(21), 6225-6228 |
| 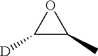 | CAS No: 34074-74-3 Supplier: Atomax (Catalog No AM34074743) Ref: Groves et al, Journal of the American Chemical Society (1986), 108(13), 3837-8 |
| 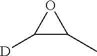 | CAS No: 34789-23-6 Ref: Watanabe et al, Polymer Journal (Tokyo, Japan) (1973), 4(6), 664-7 |
| 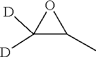 | CAS No: 89031-89-0 Ref: Stavros et al, Angewandte Chemie, International Edition (2016), 55(21), 6225-6228 |
| 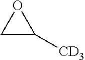 | CAS No: 2245-32-1 Supplier: https://www.alfa-chemistry.com/cas_2245-32-1 .htm + 10 other suppliers |
| 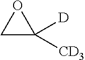 | CAS No: 1315317-85-1 Ref: Otley et al, Journal of Labelled Compounds and Radiopharmaceuticals (2011), 54(6), 308-311 |
| 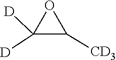 | CAS No: 1315317-84-0 Ref: Otley et al, Journal of Labelled Compounds and Radiopharmaceuticals (2011), 54(6), 308-311 |
| 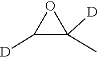 | CAS No: 59454-23-8 Ref: Michihiro et al, Makromolekulare Chemie (1976), 177(4), 1221-24 |
| 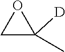 | CAS No: 41452-33-9 & 29250-75-7 Ref: Tani et al, Journal of Polymer Science, Polymer Letters Edition (1968), 6(8), 577-80 |
| 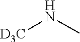 | CAS No: 120033-84-3 Supplier: 18 suppliers as HCl salt, including Sigma-Aldrich https://www.sigmaaldrich.com/GB/en/product/aldrich/486531 |
| 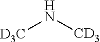 | CAS No: 53170-19-7 Supplier: 27 suppliers as HCl salt, including Sigma-Aldrich https://www.sigmaaldrich.com/GB/en/product/aldrich/279463 |
| 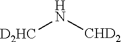 | CAS No: 35434-70-9 Ref: Mckean et al, Journal of Molecular Structure (1975), 29(1), 81-96 |
| 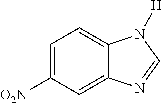 [SMILES: O=[N+]([O-])c1ccc2[nH]cnc2c1] | CAS No: 90663-68-6 Supplier: https://www.syninnova.com/catalog/product/SA-598 |
| 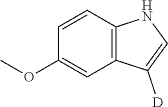 | CAS No: 1373649-66-1 Ref: Organic & Biomolecular Chemistry (2021), 19(34), 7390-7402 |

Additional materials include:

| | |
|---|---|
| 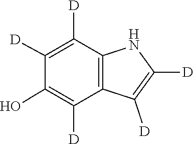 [SMILES: [2H]c1[nH]c2c([2H])c([2H])c(O)c([2H])c2c1[2H]] | CAS No: 916979-23-2 Supplier: https://www.abovchem.com/product-AC583343 Ref: Esaki et al, Tetrahedron (2006), 62(47), 10954-10961 |
| 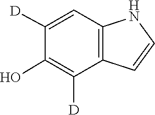 [SMILES: [2H]c1cc2[nH]ccc2c([2H])c1O] | CAS No: 132185-15-0 HRef: Hoshino et al, Agricultural and Biological Chemistry (1990), 54(9), 2339-460 |
| 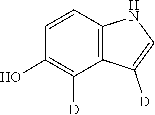 [SMILES: [2H]c1c[nH]c2ccc(O)c([2H])c12] | CAS No: 16556-01 -7 Ref: Daly et al, Journal of the American Chemical Society (1967), 89(4), 1032-3 |

### Synthesis of (R)-1-(5-cyclopropoxy-1H-indol-1-yl)-N,N-dimethylpropan-2-amine

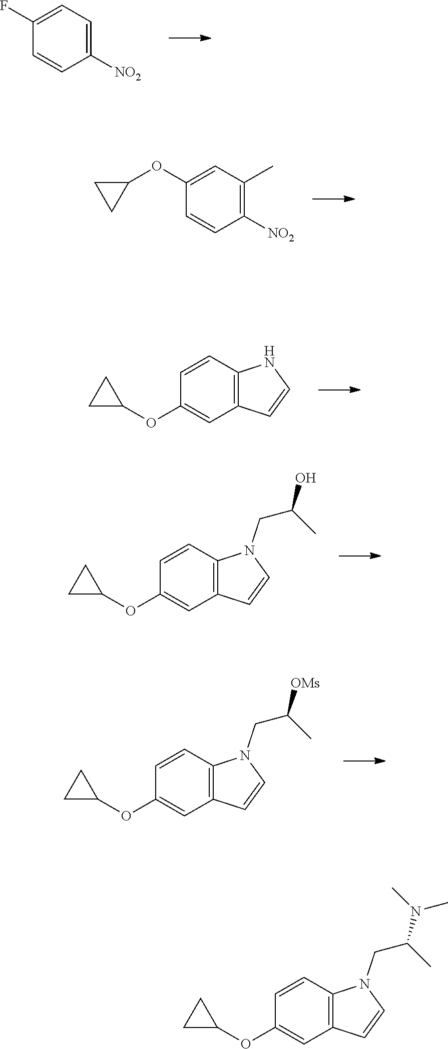 [SMILES: O=[N+]([O-])c1ccc(F)cc1 ; Cc1cc(OC2CC2)ccc1[N+](=O)[O-] ; c1cc2cc(OC3CC3)ccc2[nH]1 ; C[C@H](O)Cn1ccc2cc(OC3CC3)ccc21 ; C[C@@H](Cn1ccc2cc(OC3CC3)ccc21)OS(C)(=O)=O ; C[C@H](Cn1ccc2cc(OC3CC3)ccc21)N(C)C]

### Step 1: Preparation of 4-cyclopropoxy-2-methyl-1-nitrobenzene

To a mixture of cyclopropanol (0.55 mL, 8.6 mmol) and 4-fluoro-2-methyl-1-nitro-benzene (1.00 g, 6.5 mmol) in DMF (10 mL) was added Cs₂CO₃ (2.73 g, 8.4 mmol). The mixture was heated to 70° C. under microwave irradiation (Biotage Initiator+ microwave) and stirred for 4 h. The mixture was poured into H₂O (150 mL) and extracted with Et₂O (2×50 mL). The combined organic layers were washed with 5% LiCl (aq) and concentrated in vacuo. The crude residue was purified by column chromatography on silica gel (EtOAc/hexane 0:1 to 5:95) to afford the title compound (0.78 g, 62%) as a viscous oil. Retention time 1.83 min; m/z=194.0 [M+H]⁺; ¹H NMR (400 MHz, CDCl₃) δ 8.07 (d, J=9.0 Hz, 1H), 6.96 (ddd, J=9.0, 2.8, 0.6 Hz, 1H), 6.92 (dt, J=2.7, 0.8 Hz, 1H), 3.85-3.76 (m, 1H), 2.63 (d, J=0.6 Hz, 3H), 0.92-0.75 (m, 4H).

### Step 2: Preparation of 5-cyclopropoxy-1H-indole

A mixture of 4-cyclopropoxy-2-methyl-1-nitro-benzene (0.67 g, 3.47 mmol), 1,1-dimethoxy-N,N-dimethyl-methanamine [DMF.DMA](0.58 g, 4.86 mmol) and pyrrolidine (0.345 g, 4.86 mmol) in DMF (3 mL) was heated to 115° C. under microwave irradiation (Biotage Initiator+ microwave) and stirred for 5 h. The mixture was cooled, added to H₂O (200 mL) and extracted with Et₂O (2×60 mL). The combined organic layers were washed with 5% LiCl (aq) solution and concentrated in vacuo to afford the title compound (0.80 g, 84%) as a viscous colored oil. The crude product was used immediately in the next step without further purification.

A mixture of 10% Pd/C (66 mg), 1-[(E)-2-[5-(cyclopropoxy)-2-nitro-phenyl]vinyl]pyrrolidine (0.80 g, 3.22 mmol) in EtOAc (20 mL) was stirred under an atmosphere of H₂ (100 psi) for 19 h. The catalyst was removed by filtration through a short pad of celite and the filtrate was concentrated in vacuo. The crude residue was purified by column chromatography on silica gel (EtOAc/hexane 5:95 to 3:7) to afford the title compound (240 mg, 43%) as a viscous oil. Retention time 1.65 min; m/z=174.2 [M+H]⁺; ¹H NMR (400 MHz, CDCl₃) δ 8.03 (s, 1H), 7.35 (dt, J=2.4, 0.7 Hz, 1H), 7.27 (dt, J=8.8, 0.8 Hz, 1H), 7.18 (dd, J=3.1, 2.5 Hz, 1H), 6.89 (dd, J=8.8, 2.4 Hz, 1H), 6.49 (ddd, J=3.1, 2.0, 0.9 Hz, 1H), 3.82-3.73 (m, 1H), 0.86-0.72 (m, 4H).

### Step 3: Preparation of (2S)-1-[5-(cyclopropoxy)indol-1-yl]propan-2-ol

To a mixture of 5-(cyclopropoxy)-1H-indole (220 mg, 1.27 mmol) in DMF (4 mL) at 0° C. was added NaH, 60% in oil (58 mg, 1.52 mmol) over 3 min. The mixture was stirred at 0° C. for 30 min, then (2S)-2-methyloxirane (89 μL, 1.27 mmol) was added dropwise over 2 min. The mixture was warmed to rt and stirred overnight, then a further portion of (2S)-2-methyloxirane (45 μL, 0.64 mmol) was added and the mixture stirred at rt for 24 h. The mixture was added dropwise to ice/H₂O (50 mL) and extracted with EtOAc (3×30 mL). The combined organic layers were washed with 5% LiCl (aq) and concentrated in vacuo. The crude residue was purified by column chromatography on silica gel (EtOAc/hexane 1:4 to 1:0) to afford the title compound (138 mg, 47%) as a viscous oil. Retention time 1.61 min; m/z=232.0 [M+H]⁺; ¹H NMR (400 MHz, CDCl₃) δ 7.33 (dd, J=2.5, 0.6 Hz, 1H), 7.28-7.21 (m, 1H), 7.12 (d, J=3.1 Hz, 1H), 6.91 (ddd, J=8.9, 2.4, 0.5 Hz, 1H), 6.45 (dd, J=3.1, 0.8 Hz, 1H), 4.26-4.07 (m, 2H), 3.99 (dd, J=14.4, 7.9 Hz, 1H), 3.82-3.73 (m, 1H), 1.61 (d, J=4.0 Hz, 1H), 1.30-1.22 (m, 3H), 0.86-0.72 (m, 4H).

### Step 4: Preparation of [(1S)-2-[5-(cyclopropoxy)indol-1-yl]-1-methyl-ethyl]methanesulfonate

To a mixture of 1-[5-(cyclopropoxy)indol-1-yl]propan-2-ol (135 mg, 0.58 mmol) and DIPEA (203 μL, 1.46 mmol) in DCM (3 mL) MsCl (68 μL, 0.88 mmol) dropwise over 5 min, keeping the temperature in the range 0-5° C. The mixture was warmed to rt and stirred for 3 h, then cooled in an ice-H₂O bath, quenched by dropwise addition of H₂O (3 mL) and extracted with DCM (3×5 mL). The combined organic layers were dried over MgSO₄, filtered, and the filtrate was concentrated in vacuo to afford the title compound (195 mg, >100%) as a pale viscous oil that solidified on standing. The product was used in the next step without further purification.

Retention time 1.73 min; m/z=310.0 [M+H]⁺; ¹H NMR (400 MHz, CDCl₃) δ 7.30 (dd, J=2.4, 0.6 Hz, 1H), 7.24 (dt, J=8.9, 0.8 Hz, 1H), 7.08 (d, J=3.1 Hz, 1H), 6.94 (ddd, J=8.9, 2.4, 0.5 Hz, 1H), 6.45 (dd, J=3.1, 0.8 Hz, 1H), 4.98 (dqd, J=7.9, 6.3, 4.6 Hz, 1H), 4.33-4.18 (m, 2H), 3.80-3.71 (m, 1H), 2.26 (s, 3H), 1.49 (d, J=6.3 Hz, 3H), 0.83-0.75 (m, 4H).

### Step 5: Preparation of (2R)-1-[5-(cyclopropoxy)indol-1-yl]-N,N-dimethyl-propan-2-amine

To a mixture of [(1S)-2-[5-(cyclopropoxy)indol-1-yl]-1-methyl-ethyl]methanesulfonate (0.195 mg, 0.63 mmol) in DMF (0.7 mL) was added Me₂NH, 40% aqueous solution (2.84 g, 25.2 mmol). The mixture was heated to 65° C. under microwave irradiation (Biotage Initiator+ microwave) and stirred for 16 h. The mixture was diluted with H₂O (3 mL) and extracted into Et₂O (5×5 mL). The combined organic layers were washed with 5% aq LiCl, dried over MgSO₄, filtered and the filtrate was concentrated in vacuo. The crude residue was purified by preparative HPLC (eluent: 22-32% MeCN in 0.1% aq. formic acid gradient). Pure fractions by LCMS were combined and lyophilised to afford the title compound (50 mg, 31%) as a viscous oil. Retention time 1.23 min; m/z=259.2 [M+H]⁺; ¹H NMR (400 MHz, CDCl₃) δ 7.32 (d, J=2.4 Hz, 1H), 7.25 (d, J=8.8 Hz, 1H), 7.07 (d, J=3.1 Hz, 1H), 6.92 (dd, J=8.9, 2.4 Hz, 1H), 6.42 (dd, J=3.1, 0.8 Hz, 1H), 4.42 (dd, J=14.1, 4.8 Hz, 1H), 3.92 (dd, J=14.1, 9.1 Hz, 1H), 3.81-3.72 (m, 1H), 3.26-3.17 (m, 1H), 2.43 (s, 6H), 0.98 (d, J=6.5 Hz, 3H), 0.78 (tq, J=6.0, 2.0 Hz, 4H).

### Synthesis of (2R)—N,N-dimethyl-1-[5-(pentafluoro-)⁶-sulfanyl)indol-1-yl]propan-2-amine

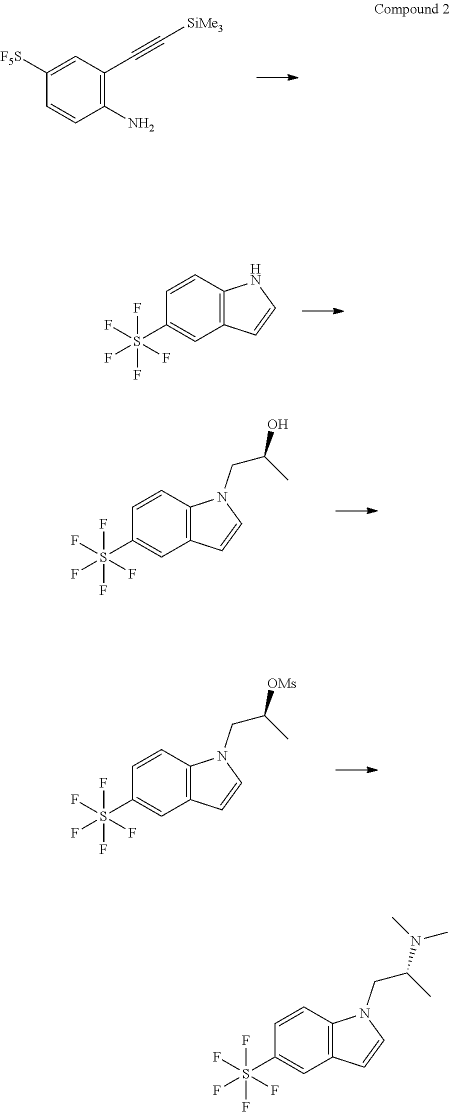

### Step 1: Preparation of pentafluoro(1H-indol-5-yl)-λ⁶-sulfane

To a mixture of 4-(pentafluoro-λ⁶-sulfanyl)-2-(2-trimethylsilylethynyl)aniline [CAS No: 1695554-02-9; WO2015049616](462 mg, 1.46 mmol) in DMF (6 mL) was added CuI (558 mg, 2.93 mmol). The mixture was stirred at rt for 10 min, then heated to 100° C. under microwave irradiation (Biotage Initiator+ microwave) and stirred for 2 h, before leaving at rt overnight. The mixture was poured into H₂O (20 mL) and extracted with Et₂O (5×5 mL). The combined organic layers were washed with 5% aq. LiCl solution (20 mL), dried over MgSO₄, filtered, and the filtrate was concentrated in vacuo. The crude residue was purified by column chromatography on silica gel (EtOAc/hexane, 1:4 to 1:1) to afford the title compound (0.211 g, 59%) as an oil. Retention time 1.75 min; m/z=242.0 [M+H]; ¹H NMR (400 MHz, DMSO-d₆) δ 7.62-7.50 (m, 2H), 6.77 (d, J=9.3 Hz, 1H), 6.20 (s, 2H), 4.46 (s, 1H).

### Step 2: Preparation of (2S)-1-[5-(Pentafluoro-λ⁶-sulfanyl)indol-1-yl]propan-2-ol

To a mixture of pentafluoro(1H-indol-5-yl)-λ⁶-sulfane (0.23 g, 0.95 mmol) in DMF (5 mL) at 0° C. was added NaH, 60% in dispersion oil (44 mg, 1.13 mmol), then (2S)-2-methyloxirane (55 mg, 0.95 mmol) fast dropwise. The mixture was warmed to rt and stirred for 72 h, then cooled in an ice-H₂O bath and quenched by the addition of H₂O (50 mL) and extracted with Et₂O (5×10 mL). The combined organic layers were washed with 5% aq. LiCl (3×50 mL), and H₂O (50 mL), dried over MgSO₄, filtered and the filtrate was concentrated in vacuo to leave a crude oil. The crude product was taken up in MeCN (10 mL), washed with hexane (2×5 mL) and concentrated in vacuo to afford the title compound (119 mg, 42%) as a colored oil, that was used in the next step without further purification. Retention time 1.74 min; m/z=302.0 [M+H]⁺; ¹H NMR (400 MHz, DMSO-d₆) δ 8.13 (d, J=2.3 Hz, 1H), 7.67 (d, J=9.2 Hz, 1H), 7.58 (dd, J=9.2, 2.4 Hz, 1H), 7.55 (d, J=3.1 Hz, 1H), 6.65 (dd, J=3.1, 0.8 Hz, 1H), 4.86 (d, J=4.9 Hz, 1H), 4.23-4.06 (m, 2H), 3.98 (tt, J=6.6, 4.8 Hz, 1H), 1.06 (d, J=6.2 Hz, 3H).

### Step 3: Preparation of [(1S)-1-Methyl-2-[5-(pentafluoro-λ⁶-sulfanyl)indol-1-yl]ethyl]methanesulfonate

To a mixture of (25)-1-[5-(pentafluoro-λ⁶-sulfanyl)indol-1-yl]propan-2-ol (120 mg, 0.40 mmol) in DCM (3 mL) at 0° C. was added DIPEA (138 μL, 0.99 mmol), followed by MsCl (46 μL, 0.60 mmol) dropwise over 20 min, in the temperature range 0-5° C. The mixture was warmed to rt and stirred for 1 h, quenched by the addition of ice-cold H₂O (1 mL), then separating and extracting with DCM (3×3 mL). The combined organic layers were dried over MgSO₄, filtered, and the filtrate was concentrated in vacuo to an colored solid. The crude product was triturated with Et₂O/hexane (1:9, 3 mL) to afford the title compound (131 mg, 87%) as a solid. Retention time 1.8 min; ¹H NMR (400 MHz, DMSO-d₆) δ 8.16 (d, J=2.3 Hz, 1H), 7.74 (d, J=9.2 Hz, 1H), 7.67-7.57 (m, 2H), 6.72 (dd, J=3.2, 0.8 Hz, 1H), 5.04 (td, J=6.4, 4.6 Hz, 1H), 4.58-4.44 (m, 2H), 2.72 (s, 3H), 1.35 (d, J=6.4 Hz, 3H).

### Step 4: Preparation of(2R)—N,N-dimethyl-1-[5-(pentafluoro-λ⁶-sulfanyl)indol-1-yl]propan-2-amine

To a mixture of [(1S)-1-methyl-2-[5-(pentafluoro-λ⁶-sulfanyl)indol-1-yl]ethyl]methanesulfonate (0.132 g, 0.35 mmol) in DMF (0.4 mL) was added Me₂NH, 40% aqueous solution (1.56 mL, 13.9 mmol). The mixture was heated to 65° C. under microwave irradiation (Biotage Initiator+ microwave) and stirred for 16 h. The mixture was cooled, diluted with H₂O (5 mL) and extracted with EtOAc (5×10 mL). The combined organic layers were washed with 5% aq. LiCl (3×25 mL), dried over MgSO₄, filtered and the filtrate was concentrated in vacuo. The crude residue was purified by preparative HPLC (eluent: 35-45% MeCN in 0.1% aq. formic acid gradient). Pure fractions by LCMS were combined and lyophilised to afford the title compound (24 mg, 21%) as a viscous oil. Retention time 1.32 min; m/z=329.0 [M+H]⁺; ¹H NMR (400 MHz, CDCl₃) δ 8.06 (d, J=2.1 Hz, 1H), 7.63 (dd, J=9.1, 2.2 Hz, 1H), 7.46 (d, J=9.2 Hz, 1H), 7.25 (d, J=3.2 Hz, 1H), 6.62 (dd, J=3.2, 0.8 Hz, 1H), 4.62 (d, J=14.2 Hz, 1H), 4.05 (dd, J=14.2, 8.8 Hz, 1H), 3.26 (s, 1H), 2.49 (s, 6H), 1.04 (d, J=6.7 Hz, 3H).

### Synthesis of (2R)-1-(8,9-dihydro-7H-pyrano[3,2-e]indol-3-yl)-N,N-dimethylpropan-2-amine

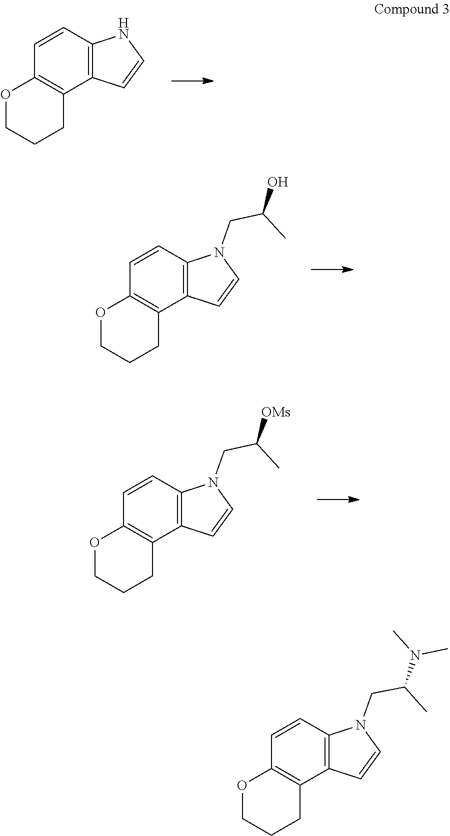 [SMILES: c1cc2c3c(ccc2[nH]1)OCCC3 ; C[C@H](O)Cn1ccc2c3c(ccc21)OCCC3 ; C[C@@H](Cn1ccc2c3c(ccc21)OCCC3)OS(C)(=O)=O ; C[C@H](Cn1ccc2c3c(ccc21)OCCC3)N(C)C]

### Step 1: Preparation of (2S)-1-(8,9-dihydro-7H-pyrano[3,2-e]indol-3-yl)propan-2-ol

To a stirred mixture of 3,7,8,9-tetrahydropyrano[3,2-e]indole [CAS No: 140427-33-4; prepared according to Tetrahedron 1992, 48, 1039-1052](19 mg, 0.11 mmol) in DMF (2 mL) was added NaH, 60% in oil (5.3 mg, 0.13 mmol) and stirred for 30 min. (2S)-2-methyloxirane (11.5 μL, 0.16 mmol) was added and the mixture stirred for 22.5 hours. A further portion of (2S)-2-methyloxirane (6 μL, 0.08 mmol) was added and the mixture stirred for 23.5 hours. A further portion of (2S)-2-methyloxirane (6 μL, 0.08 mmol) was added and the mixture stirred at rt for 24 h. A further portion of (2S)-2-methyloxirane (6 μL, 0.08 mmol) was added and the mixture stirred at rt for 71 h. The mixture was poured into H₂O (4 mL) and extracted with Et₂O (2×4 mL). The combined organic layers were washed with brine (2 mL), dried (MgSO₄), filtered and the filtrate was concentrated in vacuo. The crude residue was purified by column chromatography on silica gel (eluent: DCM) giving the title compound (13 mg, 51%) as a solid.

Retention time 3.051 min (10 minute LCMS method); m/z=[M+H]⁺ calculated for C₁₄H₁₇NO₂ 231.2; Found 232.2; ¹H NMR (400 MHz, CDCl₃) δ 7.16-7.04 (m, 2H), 6.75 (d, J=8.8 Hz, 1H), 6.39 (dd, J=3.1, 0.9 Hz, 1H), 4.27-4.05 (m, 4H), 4.04-3.89 (m, 1H), 2.94 (t, J=6.6 Hz, 2H), 2.18-2.03 (m, 2H), 1.62 (d, J=3.9 Hz, 1H), 1.25 (d, J=6.2 Hz, 3H).

### Step 2: Preparation of [(1S)-2-(8,9-dihydro-7H-pyrano[3,2-e]indol-3-yl)-1-methylethyl]methanesulfonate

To a stirred mixture of (2S)-1-(8,9-dihydro-7H-pyrano[3,2-e]indol-3-yl)propan-2-ol (13 mg, 0.056 mmol) in DCM (1 mL) was added Et₃N (10 μL, 0.07 mmol) followed by MsCl (5 μL, 0.061 mmol), stirred at rt for 18 h then at 25° C. for 24 h. Additional Et₃N (10 μL, 0.07 mmol) and MsCl (5 μL, 0.061 mmol) were added and the mixture was continued for 24 h. Additional Et₃N (20 μL, 0.14 mmol) and MsCl (10 μL, 0.07 mmol) were added and the mixture was continued for 99 h. The mixture was diluted with H₂O (3 mL) and extracted with DCM (2×3 mL). The combined organic layers were washed with brine (3 ml), dried (MgSO₄), filtered and the filtrate was concentrated in vacuo to afford the title compound (21 mg, >100%) as a solid. The crude product was used in the next step without further purification. Retention time 1.706 min; m/z=[M+H]⁺ calculated for C₁₅H₁₉NO₄S 309.3; Found 310.0; ¹H NMR (400 MHz, CDCl₃) δ 7.14-7.02 (m, 2H), 6.77 (d, J=8.9 Hz, 1H), 6.39 (dd, J=3.1, 0.9 Hz, 1H), 4.98 (dqd, J=7.5, 6.4, 4.7 Hz, 1H), 4.33-4.06 (m, 4H), 2.92 (t, J=6.6 Hz, 2H), 2.32 (s, 3H), 2.21-2.02 (m, 2H), 1.48 (d, J=6.4 Hz, 3H).

### Step 3: Preparation of (2R)-1-(8,9-dihydro-7H-pyrano[3,2-e]indol-3-yl)-N,N-dimethylpropan-2-amine

A mixture of [(1S)-2-(8,9-dihyrdo-7H-pyrano[3,2-e]indol-3-yl)-1-methylethyl]methanesulfonate (21 mg, crude material from step 2) in 40% aqueous dimethylamine (0.4 mL) was stirred at 65° C. under microwave irradiation for 16 h. The mixture was diluted with H₂O (3 mL) end extracted with DCM (2×3 mL). The combined organic layers were washed with brine (1 ml), dried (MgSO₄), filtered and concentrated in vacuo. The crude residue was purified by reverse phase preparative HPLC (eluent: 15-24% MeCN in 0.1% aq formic acid gradient). Pure fractions by LCMS were combined and lyophilised to afford the title compound (1.3 mg, 8%) as a viscous oil. Retention time 1.744 min (10 minute LCMS method); m/z=[M+H]⁺ calculated for C₁₆H₂₂N₂O 258.3; Found 259.2; ¹H NMR (400 MHz, CD₃OD) δ 7.25-7.13 (m, 2H), 6.68 (d, J=8.8 Hz, 1H), 6.42 (dd, J=3.2, 0.8 Hz, 1H), 4.51 (dd, J=14.6, 6.0 Hz, 1H), 4.31-4.09 (m, 3H), 3.72 (dp, J=8.4, 6.6 Hz, 1H), 2.91 (t, J=6.6 Hz, 2H), 2.74 (s, 6H), 2.14-1.99 (m, 2H), 1.16 (d, J=6.7 Hz, 3H).

### Synthesis of (2R)-1-(5-cyclopropylsulfonylindol-1-yl)-N,N-dimethyl-propan-2-amine

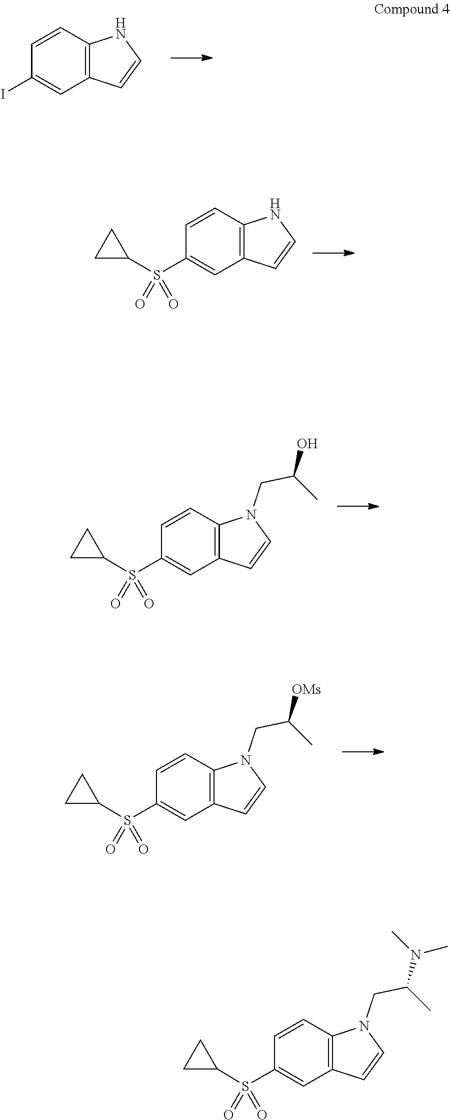 [SMILES: Ic1ccc2[nH]ccc2c1 ; O=S(=O)(c1ccc2[nH]ccc2c1)C1CC1 ; C[C@H](O)Cn1ccc2cc(S(=O)(=O)C3CC3)ccc21 ; C[C@@H](Cn1ccc2cc(S(=O)(=O)C3CC3)ccc21)OS(C)(=O)=O ; C[C@H](Cn1ccc2cc(S(=O)(=O)C3CC3)ccc21)N(C)C]

### Step 1: Preparation of 5-cyclopropylsulfonyl-1H-indole

To a mixture of 5-iodoindole (1.6 g, 6.58 mmol) in dry DMSO (10 mL) under an atmosphere of N₂ were added cyclopropylsulfinic acid, sodium salt (1.10 g, 8.56 mmol), CuI (502 mg, 2.63 mmol), L-proline (502 mg, 2.63 mmol) and NaOH (0.105 g, 2.63 mmol). The mixture was heated to 75° C. and stirred for 3 h under nitrogen. The mixture was quenched with saturated aqueous NH₄Cl (30 mL), diluted with H₂O, and extracted with EtOAc (3×50 mL). The combined organic layers were washed with H₂O and brine, dried over Na₂SO₄, filtered, and concentrated in vacuo. The crude residue was purified by column chromatography on silica gel (eluent: 20% to 100% EtOAc in hexane) to afford the title compound (132 mg, 9%) as a solid.

Retention time: 1.41 min; m/z=[M+H]⁺ calculated for C₁₁H₁₁NO₂S 222.0 found 222.0; ¹H NMR (400 MHz, CDCl₃) δ 8.66 (br. s, 1H), 8.24 (t, J=0.8 Hz, 1H), 7.69 (dd, J=8.6, 1.6 Hz, 1H), 7.51 (dt, J=8.0, 1.6 Hz, 1H), 7.36 (dd, J=3.2, 2.4 Hz, 1H), 6.7-6.68 (m, 1H), 2.53-2.46 (m, 1H), 1.38-1.33 (m, 2H), 1.02-0.98 (m, 2H).

### Step 2: (2S)-1-(6-cyclopropylsulfonylindol-1-yl)propan-2-ol

To a mixture of 6-cyclopropylsulfonyl-1H-indole (0.132 g, 0.6 mmol) in DMF (2.5 mL) was added NaH, 60% in oil at 0° C., over 20 min. The mixture was stirred for 30 min, then (S)-propylene oxide was added fast dropwise (34.6 mg, 0.6 mmol). The mixture was stirred at rt for 72 h, then H₂O (10 mL) added under ice-water bath cooling and extracted with Et₂O (5×10 mL). The combined organic layers were washed with 5% LiCl (10 mL×2), H₂O (2×10 mL), dried over MgSO₄, filtered and the filtrate was concentrated in vacuo to give the product (119 mg, 72%) as an oil. The material was taken to the next step with no further purification.

Retention time: 2.53 min (K10 method, 10 minutes run); m/z=[M+H]⁺ calculated for C₁₄H₁₇NO₃S 280.0 found 280.0; ¹H NMR (400 MHz, CDCl₃) δ 8.21 (dd, J=1.80, 0.6 Hz, 1H), 7.50 (t, J=0.8 Hz, 1H), 7.31 (d, J=3.2 Hz, 1H), 6.66 (dd, J=3.2, 0.9 Hz, 1H), 4.29-4.16 (m, 2H), 4.15-4.05 (m, 1H), 2.54-2.43 (m, 1H), 1.40-1.31 (m, 2H), 1.28 (d, J=6.1 Hz, 3H), 1.04-0.94 (m, 2H).

### Step 3: Preparation of [(1S)-2-(5-cyclopropylsulfonylindol-1-yl)-1-methyl-ethyl]methanesulfonate

To a mixture of (2S)-1-(5-cyclopropylsulfonylindol-1-yl)propan-2-ol (0.119 g, 0.426 mmol) in DCM (10 mL) was added Et₃N (0.148 mL, 1.06 mmol). The mixture was cooled to 0° C., then MsCl (0.049 mL, 0.64 mmol) dropwise over 5 min. The resulting mixture was warmed to rt and stirred overnight. The mixture was quenched by the addition of ice-cold H₂O (3 mL), then separated and the aqueous layer extracted with DCM (4×5 mL). The combined organic layers were dried over MgSO₄, filtered, and concentrated in vacuo. The residue was purified by column chromatography on silica gel (eluent: 20% to 100% EtOAc in hexane) to afford the title compound (68 mg, 37%) as an oil. ¹H NMR (400 MHz, CDCl₃) δ 8.23 (t, J=1.2 Hz, 1H), 7.76 (dd, J=8.6, 2 Hz, 1H), 7.50 (d, J=8.6 Hz, 1H), 7.28 (d, J=3.2 Hz, 1H), 6.70 (dd, J=3.2, 0.8 Hz, 1H), 5.08-5.00 (m, 1H), 4.43-4.31 (m, 2H), 2.52-2.46 (m, 4H), 1.49 (d, J=6.4 Hz, 3H), 1.38-1.35 (m, 2H), 1.20-0.99 (m, 2H).

### Step 4: Preparation of (2R)-1-(5-cyclopropylsulfonylindol-1-yl)-N,N-dimethyl-propan-2-amine

[(1S)-2-(5-cyclopropylsulfonylindol-1-yl)-1-methyl-ethyl]methanesulfonate (0.068 g, 0.190 mmol) was suspended in 40% aqueous Me₂NH (0.858 ml, 7.6 mmol) along with DMF (0.3 mL), in a microwave vial. The vial was sealed and heated to 65° C. and stirred for 16 h under microwave irradiation. The mixture was diluted with H₂O (10 mL) and extracted with Et₂O (5×10 mL). The combined organic layers were washed with 5% LiCl solution (10 mL), dried (MgSO₄), filtered and the filtrate was concentrated in vacuo. The crude residue was purified by column chromatography on silica gel (eluent: 0% to 5% MeOH in EtOAc) to afford the title compound (26 mg, 44%) as an oil. Retention time: 1.09 min; m/z=[M+H]⁺ calculated for C₁₆H₂₂N₂O₂S 307.42 found 307.42; ¹H NMR (400 Hz, CDCl₃) δ 8.21 (d, J=1.6 Hz, 1H), 7.69 (dd, J=8.8, 1.6 Hz, 1H), 7.45 (d, J=8.4 Hz, 1H), 7.26 (d, J=2.8 Hz, 1H), 6.64 (d, J=3.2 Hz, 1H), 4.31 (dd, J=14.0, 6.0 Hz, 1H), 3.98 (dd, J=14.0, 6.0 Hz, 1H), 3.09-3.02 (m, 1H), 2.52-2.46 (m, 1H), 2.34 (s, 6H), 1.37-1.34 (m, 2H), 1.00-0.97 (m, 2H), 0.93 (d, J=6.8 Hz, 3H).

### Synthesis of (2S)-1,1,1,2,3,3-hexadeuterio-3-(5-methoxyindol-1-yl)-N,N-dimethyl-propan-2-amine and (2R)-1,1,1,2,3,3-hexadeuterio-3-(5-methoxyindol-1-yl)-N,N-dimethyl-propan-2-amine

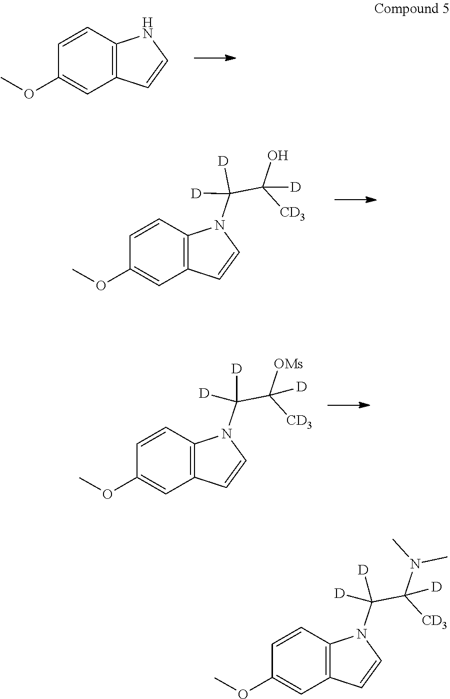

### Step 1: Preparation of (R/S) 1,1,1,2,3,3-hexadeuterio-3-(5-methoxyindol-1-yl)propan-2-ol

To a mixture of 5-methoxy-1H-indole [CAS No: 1006-94-6](0.50 g, 3.4 mmol) in DMF (5 mL) at 0° C. was added NaH, 60% in mineral oil (0.156 g, 4.1 mmol) over 20 min, forming a fine suspension. The mixture was stirred for 30 min, then 2,2,3-trideuterio-3-(trideuteriomethyl)oxirane (0.218 g, 3.4 mmol) was added dropwise. The mixture was allowed to warm to rt and stirred for 5 h, then a further portion of 2,2,3-trideuterio-3-(trideuteriomethyl)oxirane (0.218 g, 3.4 mmol) was added and the mixture was stirred at rt for 48 h. The mixture was cooled to 0° C., H₂O (70 mL) was added and the mixture was extracted with EtOAc (5×25 mL). The combined organic layers were washed with brine (3×100 mL) and H₂O (100 mL), dried over MgSO₄, filtered and the filtrate was concentrated in vacuo. The crude residue was dissolved in MeCN, washed with hexane (3×50 mL) and concentrated in vacuo to afford the title compound (0.74 g, 100%) as an oil. Retention time 1.487 min; m/z=[M+H]⁺ calculated for C₁₂H₉D₆NO₂ 212.3; found 212.2; ¹H NMR (400 MHz, CDCl₃) δ 7.25 (dt, J=9.0, 0.7 Hz, H), 7.10 (dd, J=6.5, 2.8 Hz, 2H), 6.87 (dd, J=8.9, 2.5 Hz, 1H), 6.44 (dd, J=3.1, 0.8 Hz, 1H), 3.85 (s, 3H).

### Step 2: Preparation of (R/S) [1,2,2,2-tetradeuterio-1-[dideuterio-(5-methoxyindol-1-yl)methyl]ethyl]methanesulfonate

To a mixture of 1,1,1,2,3,3-hexadeuterio-3-(5-methoxyindol-1-yl)propan-2-ol (0.718 g, 3.4 mmol) in DCM (5 mL) was added Et₃N (1.18 mL, 8.50 mmol). The mixture was cooled in an ice-water bath and MsCl (0.395 mL, 5.1 mmol) was added dropwise over 10 min. The mixture was warmed to rt and stirred for 1 h, then quenched by the addition of ice-cold H₂O (10 mL) and extracted with DCM (3×5 mL). The combined organic layers were dried over MgSO₄, filtered and the filtrate was concentrated in vacuo to an solid that was triturated with Et₂O in hexane (1:9) to give a solid that was air dried at 40° C. overnight to afford the title compound (0.75 g, 77%) as a solid. Retention time 1.7 min; ¹H NMR (400 MHz, DMSO-d₆) δ 7.38 (d, J=8.9 Hz, 1H), 7.27 (d, J=3.1 Hz, 1H), 7.03 (d, J=2.5 Hz, 1H), 6.78 (dd, J=8.9, 2.5 Hz, 1H), 6.37 (d, J=3.1 Hz, 1H).

### Step 3: Preparation of (R/S) 1,1,1,2,3,3-hexadeuterio-3-(5-methoxyindol-1-yl)-N,N-dimethyl-propan-2-amine

A mixture of [1,2,2,2-tetradeuterio-1-[dideuterio-(5-methoxyindol-1-yl)methyl]ethyl]methanesulfonate (0.184 g, 0.64 mmol) in DMF (0.4 mL) was added 40% aq. N-methylmethanamine (2.87 g, 25.4 mmol). The mixture was heated to 65° C. and stirred for 16 h under microwave irradiation, then cooled and extracted with EtOAc (3×15 mL). The combined organic layers were washed with 5% aq LiCl (3×40 mL) and H₂O (40 mL), then dried over MgSO₄, filtered and the filtrate was concentrated in vacuo. The crude residue was purified by reverse-phase preparative HPLC (eluent: 19-29% MeCN in 0.1% aq formic acid gradient). Pure fractions were combined and lyophilised to afford the title compound (48 mg, 32%) as a viscous oil. Retention time 1.12 min; m/z=[M+H]⁺ calculated for C₁₄H₁₄D₆N₂O 239.4; found 239.2; ¹H NMR (400 MHz, CDCl₃) δ 7.27 (m, 1H), 7.07 (dd, J=7.1, 2.8 Hz, 2H), 6.89 (dd, J=8.8, 2.5 Hz, 1H), 6.42 (dd, J=3.1, 0.8 Hz, 1H), 3.85 (s, 3H), 2.46 (s, 6H).

### Step 4: Chiral separation

The racemic mixture was dissolved in MeOH (3 mg/mL) and separated by preparative chiral supercritical fluid chromatography (SFC) to give (2S)-1,1,1,2,3,3-hexadeuterio-3-(5-methoxyindol-1-yl)-N,N-dimethyl-propan-2-amine (15.1 mg). Enantiomer excess, 99.8%; Retention time 1.19 min; m/z=[M+H]⁺ calculated for C₁₄H₁₄D₆N₂O 239.4; found 239.2; ¹H NMR (400 MHz, CD₃OD) δ 7.31 (dt, J=8.9, 0.7 Hz, 1H), 7.17 (dd, J=3.1, 0.5 Hz, 1H), 7.07 (dd, J=2.5, 0.6 Hz, 1H), 6.83 (ddd, J=8.9, 2.5, 0.5 Hz, 1H), 6.38 (dd, J=3.1, 0.9 Hz, 1H), 3.82 (s, 3H), 2.39 (s, 6H). Also obtained from the preparative chiral SFC column was (2R)-1,1,1,2,3,3-hexadeuterio-3-(5-methoxyindol-1-yl)-N,N-dimethyl-propan-2-amine (15.7 mg).

Enantiomer excess, 99.2%; Retention time 1.19 min; m/z=[M+H]⁺ calculated for C₁₄H₁₄D₆N₂O 239.4; found 239.2; ¹H NMR (400 MHz, CD₃OD) δ 7.31 (d, J=8.9 Hz, 1H), 7.17 (d, J=3.1 Hz, 1H), 7.07 (d, J=2.4 Hz, 1H), 6.83 (dd, J=8.9, 2.5 Hz, 1H), 6.38 (dd, J=3.1, 0.8 Hz, 1H), 3.82 (s, 3H), 2.39 (s, 6H).

### Synthesis of (2S)-1,1,1,2,3,3-hexadeuterio-3-(5-methoxyindol-1-yl)-N,N-bis(trideuteriomethyl)propan-2-amine and (2R)-1,1,1,2,3,3-hexadeuterio-3-(5-methoxyindol-1-yl)-N,N-bis(trideuteriomethyl)propan-2-amine

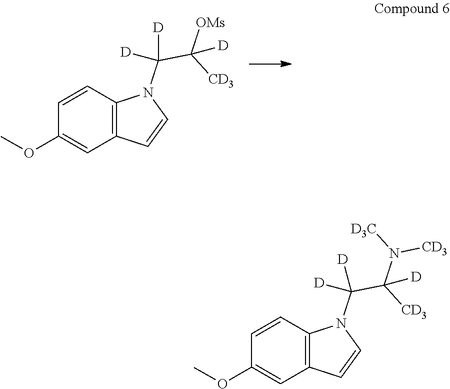 [SMILES: [2H]C([2H])([2H])C([2H])(OS(C)(=O)=O)C([2H])([2H])n1ccc2cc(OC)ccc21 ; [2H]C([2H])([2H])N(C([2H])([2H])[2H])C([2H])(C([2H])([2H])[2H])C([2H])([2H])n1ccc2cc(OC)ccc21]

### Step 1: Preparation of (R/S)-1,1,1,2,3,3-hexadeuterio-3-(5-methoxyindol-1-yl)-N,N-bis(trideuteriomethyl)propan-2-amine

1,1,1-Trideuterio-N-(trideuteriomethyl)methanamine HCl (0.908 g, 10.0 mmol) was suspended in H₂O (1.32 mL) and basified with NaOH (0.415 g, 10.0 mmol) to give a 40% free base aqueous solution. The free base was added to a mixture of [1,2,2,2-tetradeuterio-1-[dideuterio-(5-methoxyindol-1-yl)methyl]ethyl]methanesulfonate (0.10 g, 0.35 mmol) in DMF (0.44 mL). The mixture was heated to 65° C. and stirred for 16 h under microwave irradiation, then cooled and extracted with EtOAc (3×15 mL). The combined organic layers were washed with 5% LiCl aq (3×40 mL) and H₂O (40 mL), dried over MgSO₄, filtered and the filtrate was concentrated in vacuo. The crude residue was purified by reverse-phase preparative HPLC (eluent: 10-100% CH₃CN in water. The fractions were lyophilised to afford the title compound (19 mg, 22%) as an oil. Retention time 1.13 min; m/z=[M+H]⁺ calculated for C₁₄H₈D₁₂N₂O 245.4; found 245.2; ¹H NMR (400 MHz, CDCl₃) δ 7.24 (m, 1H), 7.08 (ddd, J=7.5, 2.8, 0.5 Hz, 2H), 6.87 (ddd, J=8.9, 2.5, 0.5 Hz, 1H), 6.41 (dd, J=3.1, 0.9 Hz, 1H), 3.85 (s, 3H).

### Step 2: Chiral separation

The racemic mixture was dissolved in MeOH (3 mg/mL) and separated by preparative chiral supercritical fluid chromatography (SFC) to give (2S)-1,1,1,2,3,3-hexadeuterio-3-(5-methoxyindol-1-yl)-N,N-bis(trideuteriomethyl)propan-2-amine (10 mg), which contains 12% of the non-deuterated dimethylamine analogue. Enantiomer excess, 99.9%; Retention time 1.19 min; m/z=[M+H]⁺ calculated for C₁₄H₈D₁₂N₂O 245.4; found 245.2; ¹H NMR (400 MHz, CD₃OD) δ 7.29 (d, J=8.9, 1H), 7.15 (d, J=3.1 Hz, 1H), 7.05 (d, J=2.5 Hz, 1H), 6.81 (dd, J=8.9, 2.5 Hz, 1H), 6.36 (dd, J=3.0, 0.9 Hz, 1H), 3.80 (s, 3H). Also obtained from the preparative chiral SFC column was (2R)-1,1,1,2,3,3-hexadeuterio-3-(5-methoxyindol-1-yl)-N,N-bis(trideuteriomethyl)propan-2-amine (9.8 mg), which contains 11% of the non-deuterated dimethylamine analogue. Enantiomer excess, 98.9%; Retention time 1.123 min; m/z=[M+H]⁺ calculated for C₁₄H₈D₁₂N₂O 245.4; found 245.2; ¹H NMR (400 MHz, CD₃OD) δ 7.31 (d, J=8.9 Hz, 1H), 7.18 (d, J=3.1 Hz, 1H), 7.08 (d, J=2.4 Hz, 1H), 6.84 (dd, J=8.9, 2.4 Hz, 1H), 6.39 (dd, J=3.1, 0.9 Hz, 1H), 3.82 (s, 3H).

### Synthesis of (2S)-1,1,1,2,3,3-hexadeuterio-N,N-dimethyl-3-[5-(trideuteriomethoxy)indol-1-yl]propan-2-amine and (2R)-1,1,1,2,3,3-hexadeuterio-N,N-dimethyl-3-[5-(trideuteriomethoxy)indol-1-yl]propan-2-amine

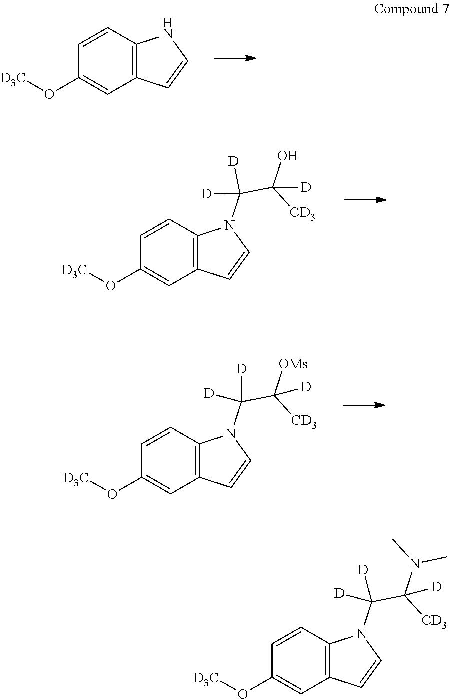 [SMILES: [2H]C([2H])([2H])Oc1ccc2[nH]ccc2c1 ; [2H]C([2H])([2H])Oc1ccc2c(ccn2C([2H])([2H])C([2H])(O)C([2H])([2H])[2H])c1 ; [2H]C([2H])([2H])Oc1ccc2c(ccn2C([2H])([2H])C([2H])(OS(C)(=O)=O)C([2H])([2H])[2H])c1 ; [2H]C([2H])([2H])Oc1ccc2c(ccn2C([2H])([2H])C([2H])(N(C)C)C([2H])([2H])[2H])c1]

### Step 1: Preparation of(R/S)-1,1,1,2,3,3-hexadeuterio-3-[5-(trideuteriomethoxy)indol-1-yl]propan-2-ol

To a mixture of 5-(trideuteriomethoxy)-1H-indole [CAS No: 90663-68-6](0.256 g, 1.70 mmol) in DMF at 0° C. was added NaH, 60% in mineral oil (78 mg, 2.05 mmol) over 20 min, forming a thick suspension. The mixture was stirred for 30 min then 2,2,3-trideuterio-3-(trideuteriomethyl)oxirane (0.109 g, 1.7 mmol) was added dropwise. The mixture was allowed to warm to rt and stirred for 120 h, adding an extra portion of 2,2,3-trideuterio-3-(trideuteriomethyl) oxirane (55 mg, 1.03 mmol) after 48 h. The reaction was cooled in an ice-H₂O bath, quenched with H₂O (70 mL) and extracted with EtOAc (5×20 mL). The combined organic layers were washed with 5% LiCl aq (3×50 mL) and H₂O (50 mL), dried over MgSO₄, filtered, and the filtrate was concentrated in vacuo. The crude residue was dissolved in MeCN (30 mL), washed with hexane (3×20 mL) and concentrated in vacuo to afford the title compound (0.136 g, 37%) as an oil. Retention time 1.488 min; m/z=[M+H]⁺ calculated for C₁₂H₆D₉NO₂ 215.3; found 215.2; ¹H NMR (400 MHz, DMSO-d₆) δ 7.35 (dt, J=8.9, 0.7 Hz, 1H), 7.26 (dd, J=3.0, 0.5 Hz, 1H), 7.02 (dd, J=2.4, 0.6 Hz, 1H), 6.75 (ddd, J=8.9, 2.5, 0.5 Hz, 1H), 6.30 (dd, J=3.0, 0.8 Hz, 1H), 4.75 (s, 1H).

### Step 2: Preparation of (R/S)-1,2,2,2-tetradeuterio-1-[dideuterio-[5-(trideuteriomethoxy)indol-1-yl]methyl]ethyl]methanesulfonate

To a mixture of 1,1,1,2,3,3-hexadeuterio-3-[5-(trideuteriomethoxy)indol-1-yl]propan-2-ol (0.135 g, 0.63 mmol) in DCM (3 mL) was added Et₃N (0.22 mL, 1.57 mmol). The mixture was cooled in an ice-H₂O bath and MsCl (0.073 mL, 0.95 mmol) was added dropwise. The mixture was warmed to rt and stirred for 1 h, then quenched by the addition of ice-cold H₂O (5 mL) and extracted with DCM (3×5 mL). The combined organic layers were dried over MgSO₄, filtered, and the filtrate was concentrated in vacuo to leave a solid, that was triturated with Et₂O in hexane (1:9) to give a solid, that was air dried at 40° C. overnight to afford the title compound (0.192 g, 100%) as a solid. Retention time 1.63 min; m/z=[M+H]⁺ calculated for C₁₃H₈D₉NO₄S 293.4; found 293.1; ¹H NMR (400 MHz, DMSO-d₆) δ 7.44 (dt, J=8.9, 0.7 Hz, 1H), 7.32 (d, J=3.0 Hz, 1H), 7.04 (dd, J=2.5, 0.6 Hz, 1H), 6.80 (dd, J=8.9, 2.5 Hz, 1H), 6.38 (dd, J=3.1, 0.9 Hz, 1H), 2.56 (s, 3H).

### Step 3: Preparation of (R/S)-1,1,1,2,3,3-hexadeuterio-N,N-dimethyl-3-[5-(trideuteriomethoxy)indol-1-yl]propan-2-amine

To a mixture of 1,2,2,2-tetradeuterio-1-[dideuterio-[5-(trideuteriomethoxy)indol-1-yl]methyl]ethyl]methanesulfonate in DMF (0.5 mL) was added 40% aq. N-methylmethanamine (2.98 g, 26.4 mmol). The mixture was heated to 65° C. and stirred for 16 h under microwave irradiation, then cooled and extracted with EtOAc (3×15 mL). The combined organic layers were washed with 5% aq LiCl (3×40 mL), dried over MgSO₄, filtered and the filtrate was concentrated in vacuo. The crude residue was purified by reverse-phase preparative HPLC (eluent: 15-25% MeCN in 0.1% aq formic acid). The fractions were lyophilised to afford the title compound (45 mg, 28%) as a viscous oil. Retention time 1.112 min; m/z=[M+H]⁺ calculated for C₁₄H₈D₁₂N₂O 241.4; found 242.2; ¹H NMR (400 MHz, CDCl₃) δ 7.37 (dt, J=8.9, 0.7 Hz, 1H), 7.14-7.03 (m, 2H), 6.91 (ddd, J=8.9, 2.5, 0.5 Hz, 1H), 6.45 (dd, J=3.1, 0.8 Hz, 1H), 2.63 (s, 7H).

### Step 4: Chiral separation

The racemic mixture was dissolved in MeOH (3 mg/mL) and separated by preparative chiral supercritical fluid chromatography (SFC) to give (2S)-1,1,1,2,3,3-hexadeuterio-N,N-dimethyl-3-[5-(trideuteriomethoxy)indol-1-yl]propan-2-amine (8.9 mg). Enantiomer excess, 99.9%.

Retention time 1.11 min; m/z=[M+H]'⁰ calculated for C₁₄H₈D₁₂N₂O 241.4; found 242.2; ¹H NMR (400 MHz, CD₃OD) δ 7.28 (dt, J=8.9, 0.7 Hz, 1H), 7.15 (d, J=3.1 Hz, 1H), 7.05 (dd, J=2.5, 0.6 Hz, 1H), 6.81 (dd, J=8.9, 2.5 Hz, 1H), 6.36 (dd, J=3.1, 0.8 Hz, 1H), 2.36 (s, 6H). Also obtained from the preparative chiral SFC column was (2R)-1,1,1,2,3,3-hexadeuterio-N,N-dimethyl-3-[5-(trideuteriomethoxy)indol-1-yl]propan-2-amine (7.3 mg) Compound 151.

Enantiomer excess, 98.2%. Retention time 1.18 min; m/z=[M+H]'⁰ calculated for C₁₄H₈D₁₂N₂O 241.4; found 242.2; ¹H NMR (400 MHz, CD₃OD) δ 7.29 (dt, J=8.9, 0.7 Hz, 1H), 7.16 (d, J=3.1 Hz, 1H), 7.06 (dd, J=2.5, 0.6 Hz, 1H), 6.82 (dd, J=8.9, 2.4 Hz, 1H), 6.37 (dd, J=3.1, 0.9 Hz, 1H), 2.41 (s, 6H).

### Synthesis of (2S)-1,1,1,2,3,3-hexadeuterio-3-[5-(trideuteriomethoxy)indol-1-yl]-N,N-bis(trideuteriomethyl) propan-2-amine and (2R)-1,1,1,2,3,3-hexadeuterio-3-[5-(trideuteriomethoxy)indol-1-yl]-N,N-bis(trideuteriomethyl) propan-2-amine

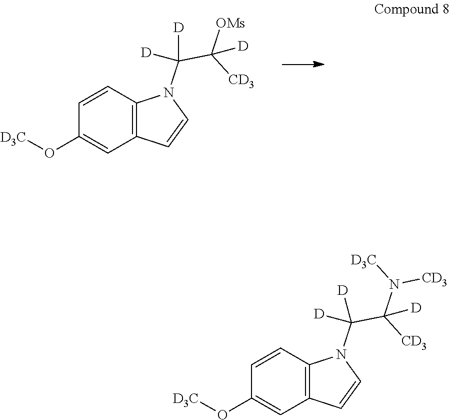 [SMILES: [2H]C([2H])([2H])Oc1ccc2c(ccn2C([2H])([2H])C([2H])(OS(C)(=O)=O)C([2H])([2H])[2H])c1 ; [2H]C([2H])([2H])Oc1ccc2c(ccn2C([2H])([2H])C([2H])(N(C([2H])([2H])[2H])C([2H])([2H])[2H])C([2H])([2H])[2H])c1]

### Step 1: Preparation of (R/S)-1,1,1,2,3,3-hexadeuterio-3-[5-(trideuteriomethoxy)indol-1-yl]-N,N-bis(trideuteriomethyl) propan-2-amine

1,1,1-Trideuterio-N-(trideuteriomethyl)methanamine hydrochloride (1.46 g, 16.7 mmol) was suspended in H₂O (2.15 mL) and basified with NaOH (669 mg, 16.7 mmol) to give a 40% aqueous solution of the free base. The free base was added to a mixture of 1,2,2,2-tetradeuterio-1-[dideuterio-[5-(trideuteriomethoxy)indol-1-yl]methyl]ethyl]methanesulfonate (0.163 g, 0.56 mmol) in DMF (0.74 mL), heated to 65° C. and stirred for 16 h under microwave irradiation. The mixture was cooled and extracted with EtOAc (3×15 mL). The combined organic layers were washed with 5% LiCl aq (3×40 mL) and H₂O (40 mL), dried over MgSO₄, filtered and the filtrate was concentrated in vacuo. The crude residue was purified by reverse-phase preparative HPLC (eluent: 60-69% MeCN in water). Pure fractions were combined and lyophilised to afford the title compound (40 mg, 29%) as a viscous oil. Retention time 1.116 mins; m/z=[M+H]⁺ calculated for C₁₄H₈D₁₂N₂O 248.4; found 248.2; ¹H NMR (400 MHz, CDCl₃) δ 7.23 (dt, J=8.9, 0.7 Hz, 1H), 7.07 (ddd, J=7.4, 2.8, 0.5 Hz, 2H), 6.86 (ddd, J=8.9, 2.4, 0.5 Hz, 1H), 6.40 (dd, J=3.1, 0.8 Hz, 1H).

### Step 2: Chiral separation

The racemic mixture was dissolved in MeOH (3 mg/mL) and separated by preparative chiral supercritical fluid chromatography (SFC) to give (2S)-1,1,1,2,3,3-hexadeuterio-3-[5-(trideuteriomethoxy)indol-1-yl]-N,N-bis(trideuteriomethyl) propan-2-amine (10.1 mg), which contains 9% of the non-deuterated dimethylamine analogue. Enantiomer excess >99.6%; Retention time 1.122 min; m/z=[M+H]⁺ calculated for C₁₄H₈D₁₂N₂O 248.4; found 248.2; ¹H NMR (400 MHz, CD₃OD) δ 7.29 (dt, J=8.9, 0.7 Hz, 1H), 7.15 (d, J=3.1 Hz, 1H), 7.05 (dd, J=2.5, 0.6 Hz, 1H), 6.81 (dd, J=8.9, 2.4 Hz, 1H), 6.36 (dd, J=3.0, 0.9 Hz, 1H). Also obtained from the preparative chiral SFC column was (2R)-1,1,1,2,3,3-hexadeuterio-3-[5-(trideuteriomethoxy)indol-1-yl]-N,N-bis(trideuteriomethyl) propan-2-amine (13.4 mg) Compound 152, which contains 18% of the non-deuterated dimethylamine analogue.

Enantiomer excess 99.2%; Retention time 1.16 min; m/z=[M+H]⁺ calculated for C₁₄H₈D₁₂N₂O 248.4; found 248.2; ¹H NMR (400 MHz, CD₃OD) δ 7.30 (dt, J=8.9, 0.7 Hz, 1H), 7.17 (dd, J=3.1, 0.5 Hz, 1H), 7.07 (dd, J=2.5, 0.6 Hz, 1H), 6.83 (ddd, J=8.9, 2.5, 0.5 Hz, 1H), 6.38 (dd, J=3.0, 0.8 Hz, 1H).

### Synthesis of (2R)—N,N-dimethyl-1-(5-methylsulfanylindol-1-yl)propan-2-amine

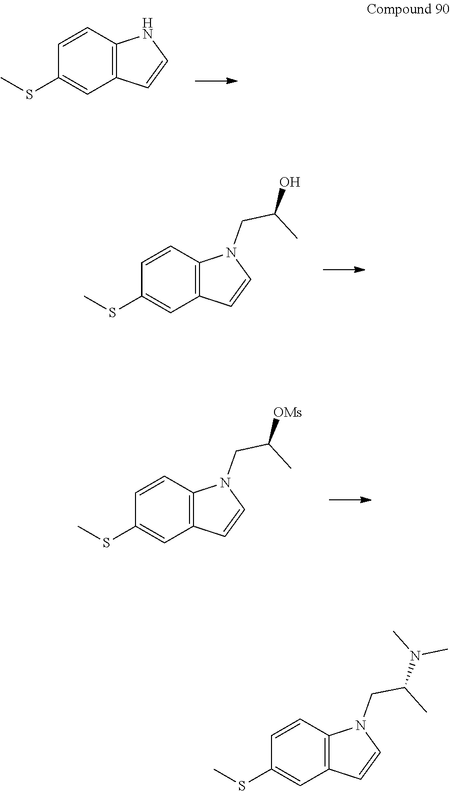

### Step 1: Preparation of (2S)-1-[5-(methylsulfanyl)-1H-indol-1-yl]propan-2-ol

To a stirred mixture of 5-methylthio-1H-indole [CAS No: 77248-65-8](0.450 g, 2.76 mmol) in DMF (4.5 mL) was added NaH, 60% in oil (0.127 g, 3.31 mmol) at 0° C., over 20 min. The mixture was stirred for 30 min, then (S)-propylene oxide (0.160 g, 2.76 mmol) was added fast dropwise. The mixture was stirred at rt for 72 h, then quenched with the addition of H₂O (5 mL) under ice/water bath cooling and extracted into EtOAc (5×5 mL). The combined organic layers were washed with brine (3×10 mL), H₂O (10 mL), dried over MgSO₄, filtered, and concentrated to an oil. The crude oil was taken back up in CH₃CN, washed with hexane and concentrated in vacuo to afford the title compound (489 mg, 80%) as an oil. Retention time 1.64 min; m/z=[M+H]⁺ calculated for C₁₂H₁₅NOS 222.3; found 222.0; ¹H NMR (400 MHz, CDCl₃) δ 7.63 (dd, J=1.8, 0.7 Hz, 1H), 7.36-7.20 (m, 2H), 7.13 (d, J=3.1 Hz, 1H), 6.46 (dd, J=3.1, 0.8 Hz, 1H), 4.27-3.93 (m, 3H), 2.51 (s, 3H), 1.25 (d, J=6.2 Hz, 3H).

### Step 2: Preparation of [(1S)-1-methyl-2-(5-methylsulfanylindol-1-yl)ethyl]methanesulfonate

To a stirred mixture of (2S)-1-[5-(methylsulfanyl)-1H-indol-1-yl]propan-2-ol (0.439 g, 1.98 mmol) in DCM (5 mL) was added Et₃N (0.691 mL, 4.96 mmol). The solution was cooled to 0° C., then MsCl (0.23 mL, 2.98 mmol) was added dropwise over 20 min, in the temperature range 0-5° C. The resulting mixture was warmed to rt and stirred for 1 h, then quenched by the addition of ice-cold H₂O before separating and extracting the aqueous into DCM. The combined organic layers were dried over MgSO₄, filtered, and concentrated in vacuo to leave a crude oil, which was triturated with Et₂O/hexane (1:9) to afford the title compound (450 mg, 76%) as an oil that very slowly solidified on standing. Retention time 1.718 min; m/z=[M+H]⁺ calculated for C₁₃H₁₇NO₃S₂ 300.0; found 300.0; ¹H NMR (400 MHz, CDCl₃) δ 7.59 (dd, J=1.7, 0.7 Hz, 1H), 7.35-7.20 (m, 2H), 7.11 (d, J=3.2 Hz, 1H), 6.47 (dd, J=3.2, 0.8 Hz, 1H), 4.98 (dqd, J=7.7, 6.4, 4.5 Hz, 1H), 4.37-4.22 (m, 2H), 2.51 (s, 3H), 2.29 (s, 3H), 1.49 (d, J=6.3 Hz, 3H).

### Step 3: Preparation of (2R)—N,N-dimethyl-1-(5-methylsulfanylindol-1-yl)propan-2-amine

A microwave vial was charged with [(1S)-1-methyl-2-(5-methylsulfanylindol-1-yl)ethyl]methanesulfonate (250 mg, 0.84 mmol), 40% aq. Me₂NH (3.76 mL, 33.4 mmol) and DMF (0.8 mL). The mixture was sealed in a microwave vial before being heated to 65° C. and stirred for 16 h in a Biotage Initiator+ microwave. The mixture was extracted with EtOAc (3×10 mL), and the combined organic layers were washed with brine (3×10 mL), dried over MgSO₄, filtered, and concentrated in vacuo to an oil. The crude material was purified by column chromatography on silica gel(eluent: 0-20% EtOAc in MeOH) to afford the title compound (88 mg, 43%) as an oil.

Retention time 1.247 min; m/z=[M+H]⁺ calculated for C₁₄H₂₀N₂S 249.4; found 249.2; ¹H NMR (400 MHz, DMSO-d₆) δ 7.50 (dd, J=1.8, 0.6 Hz, 1H), 7.42 (dt, J=8.6, 0.8 Hz, 1H), 7.34 (d, J=3.1 Hz, 1H), 7.11 (dd, J=8.5, 1.8 Hz, 1H), 6.36 (dd, J=3.1, 0.9 Hz, 1H), 4.20 (dd, J=14.2, 6.9 Hz, 1H), 4.01 (ddd, J=16.3, 14.2, 7.2 Hz, 1H), 2.99 (h, J=6.7 Hz, 1H), 2.46 (s, 3H), 2.20 (s, 6H), 0.82 (d, J=6.7 Hz, 3H).

### Synthesis of (R/S) 1-(7-methoxyimidazo[1,2-a]pyridin-3-yl)-N,N-dimethyl-propan-2-amine

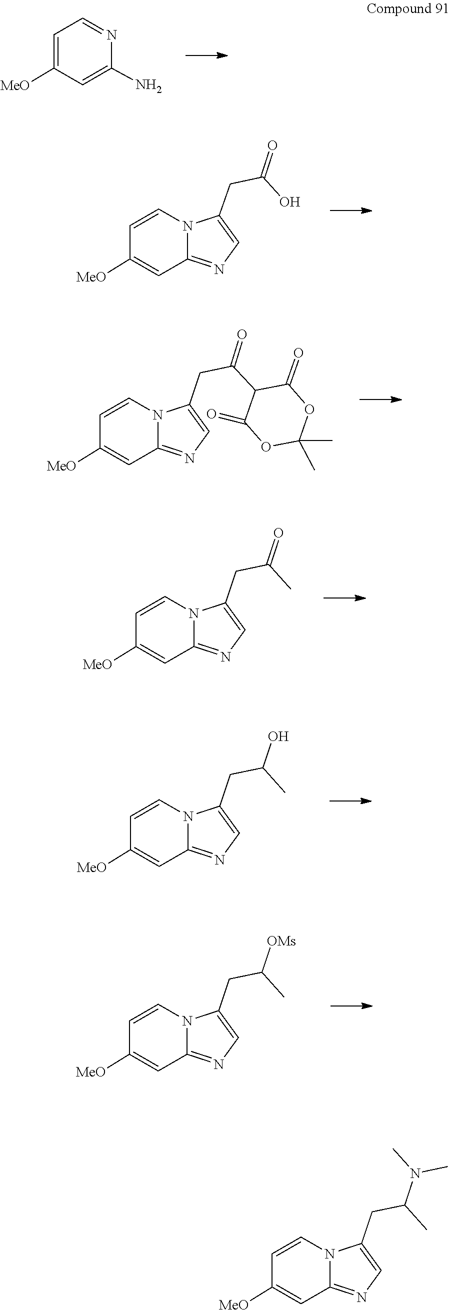

### Step 1: Preparation of 2-(7-methoxyimidazo[1,2-a]pyridin-3-yl)acetic acid

To a stirred mixture of 4-methoxypyridin-2-amine (1.32 g, 10.6 mmol) in H₂O (13 mL) at rt was added ethyl (E)-4-oxobut-2-enoate (1.32 mL, 10.9 mmol) dropwise over 3 min. The mixture was stirred for 1 h, then a solution of KOH (0.80 g, 14.3 mmol) in H₂O (1.5 mL) was added dropwise over 1 min. The mixture was stirred for 1 h at rt and was then adjusted to pH ˜5 by dropwise addition of 2M HCl. The mixture was stirred overnight and the precipitate was isolated by filtration, washed with H₂O (2×3 mL) and IMS (3 mL) and dried overnight under vacuum at 40° C. to give the title compound (0.42 g, 19%) as a solid. Retention time 0.46 min; m/z=[M+H]⁺ calculated for C₁₀H₁₀N₂O₃ 207.0; found 207.0; ¹H NMR (400 MHz, DMSO-d₆) δ 8.15 (dd, J=7.6, 0.7 Hz, 1H), 7.28 (s, 1H), 6.91 (d, J=2.4 Hz, 1H), 6.65 (dd, J=7.5, 2.6 Hz, 1H), 3.94 (s, 2H), 3.83 (s, 3H).

### Step 2: Preparation of 5-[2-(7-methoxyimidazo[1,2-a]pyridin-3-yl)acetyl]-2,2-dimethyl-1,3-dioxane-4,6-dione

To a mixture of 2-(7-methoxyimidazo[1,2-a]pyridin-3-yl)acetic acid (754 mg, 3.66 mmol) in DMF (10 ml) at rt was added DMAP (670 mg, 5.49 mmol) and 2,2-dimethyl-1,3-dioxane-4,6-dione (580 mg, 4.02 mmol). N,N′-dicyclohexylmethanediimine (830 mg, 4.02 mmol) was added portion wise over 5 min and the mixture was stirred at rt overnight. The mixture was diluted with brine (100 ml) and extracted with 1:1 IPA-chloroform (5×30 mL). The combined organic layers were concentrated in vacuo to an oil, then purified by column chromatography on silica gel (eluent: 2-10% MeOH (containing 2% ammonia “880”) in DCM. No pure fractions were obtained, so product-rich fractions were combined and concentrated in vacuo to afford the title compound (0.61 g, 50%) as an oil that solidified on standing. This material was used in the next step without further purification. Retention time 1.099 min; m/z=[M+H]⁺ calculated for C₁₆H₁₆N₂O₆ 333.1; found 333.0; ¹H NMR (400 MHz, DMSO-d₆) δ 8.34 (d, J=7.5 Hz, 1H), 7.65 (s, 1H), 7.19 (d, J=2.4 Hz, 1H), 7.01 (d, J=7.3 Hz, 1H), 4.46 (s, 2H), 3.95 (s, 3H), 1.54 (s, 6H).

### Step 3: Preparation of 1-(7-methoxyimidazo[1,2-a]pyridin-3-yl)propan-2-one

To a mixture of 5-[2-(7-methoxyimidazo[1,2-a]pyridin-3-yl)acetyl]-2,2-dimethyl-1,3-dioxane-4,6-dione (500 mg, 1.5 mmol) in 1,4-dioxane (7.5 mL) was added AcOH (1.25 mL). The mixture was heated to 100° C. under microwave irradiation and stirred for 30 min in a Biotage initiator+ microwave. The mixture was combined with that of a validation batch and concentrated in vacuo. The crude product was purified by column chromatography on silica gel (eluent 2-15% MeOH (with 2% ammonia “880” solution) in DCM. Unfortunately, no pure fractions were obtained, so product-rich fractions were combined to afford the title compound (51 mg, 13% over two batches) as a viscous oil. This material was used in the next step without further purification. Retention time 0.722 min; m/z=[M+H]⁺ calculated for C₁₁H₁₂N₂O₂ 205.0; found 205.0; ¹H NMR (400 MHz, CDCl₃) δ 7.75 (dd, J=7.5, 0.7 Hz, 1H), 7.39 (d, J=0.9 Hz, 1H), 6.88 (dd, J=2.5, 0.7 Hz, 1H), 6.55 (dd, J=7.5, 2.5 Hz, 1H), 3.92 (s, 2H), 3.86 (s, 3H), 2.19 (s, 3H).

### Step 4: Preparation of (R/S) 1-(7-methoxyimidazo[1,2-a]pyridin-3-yl)propan-2-ol

To a mixture of 1-(7-methoxyimidazo[1,2-a]pyridin-3-yl)propan-2-one (51 mg, 0.25 mmol) in MeOH, cooled in an ice-water bath, was added NaBH₄ (10.4 mg, 0.275 mmol) portion wise over 2 min. The mixture was warmed to rt and stirred for 3 h, then diluted with EtOAc (20 Ll) and washed with brine (2 mL). The organic layer was concentrated in vacuo to afford the title compound (33 mg, 64%) as an oil that was used in the next step without further purification.

Retention time 0.898 min; m/z=[M+H]⁺ calculated for C₁₁H₁₄N₂O₂ 207.1; found 207.0; ¹H NMR (400 MHz, CDCl₃) δ 7.92 (dd, J=7.5, 0.7 Hz, 1H), 7.30 (s, 1H), 6.84 (d, J=2.5 Hz, 1H), 6.53 (dd, J=7.5, 2.5 Hz, 1H), 4.15 (dqd, J=7.5, 6.2, 4.5 Hz, 1H), 3.85 (s, 3H), 3.09-2.85 (m, 2H), 1.32 (d, J=6.2 Hz, 3H).

### Step 5: Preparation of (R/S) [2-(7-methoxyimidazo[1,2-a]pyridin-3-yl)-1-methyl-ethyl]methanesulfonate

To a mixture of (R/S) 1-(7-methoxyimidazo[1,2-a]pyridin-3-yl)propan-2-ol (33 mg, 0.16 mmol) in DCM (1 mL) in an ice/water bath was added Et₃N (55.8 μL, 0.40 mmol) followed by MsCl (18.6 μL, 0.24 mmol). The mixture was warmed to rt and stirred overnight, then diluted with DCM (10 mL) and washed with concentrated sodium bicarbonate solution (3 mL). The organic layer was concentrated in vacuo to afford the title compound (45 mg, 98%) which was used in the next step without further purification. Retention time 1.018 min; m/z=[M+H]⁺ calculated for C₁₂H₁₆N₂O₄S 285.0; found 285.0; ¹H NMR (400 MHz, CDCl₃) δ 8.06 (d, J=7.6 Hz, 1H), 7.42 (s, 1H), 7.23 (s, 1H), 6.75 (dd, J=7.5, 2.5 Hz, 1H), 5.02 (q, J=6.2 Hz, 1H), 3.92 (s, 3H), 3.67 (p, J=6.6 Hz, 2H), 2.81 (s, 3H), 1.26 (s, 3H).

### Step 6: Preparation of (R/S) 1-(7-methoxyimidazo[1,2-a]pyridin-3-yl)-N,N-dimethyl-propan-2-amine

To a microwave vial charged with a mixture (R/S) [2-(7-methoxyimidazo[1,2-a]pyridin-3-yl)-1-methyl-ethyl]methanesulfonate (45 mg, 0.16 mmol) in DMF (0.2 mL) was added 40% aq Me₂NH (714 mg, 6.33 mmol). The mixture was heated under microwave irradiation to 65° C. and stirred for 12 h in a Biotage Initiator+ microwave, then purified by preparative HPLC (eluent: 0 to 30% MeCN in water gradient) to afford the title compound (0.30 mg, 0.8%) as a viscous oil.

Retention time 4.050 min; m/z=[M+H]⁺ calculated for C₁₃H₁₉N₃O 234.1; found 234.2; ¹H NMR (400 MHz, CD₃OD) δ 8.21 (d, J=7.4 Hz, 1H), 7.36 (s, 1H), 6.90 (d, J=2.5 Hz, 1H), 6.75 (dd, J=7.5, 2.5 Hz, 1H), 3.90 (d, J=1.7 Hz, 3H), 3.51-3.47 (m, 1H), 2.99 (s, 2H), 2.70 (s, 6H).

### Synthesis of (R/S) 1-(7-methoxyimidazo[1,5-a]pyridin-3-yl)-N,N-dimethyl-propan-2-amine

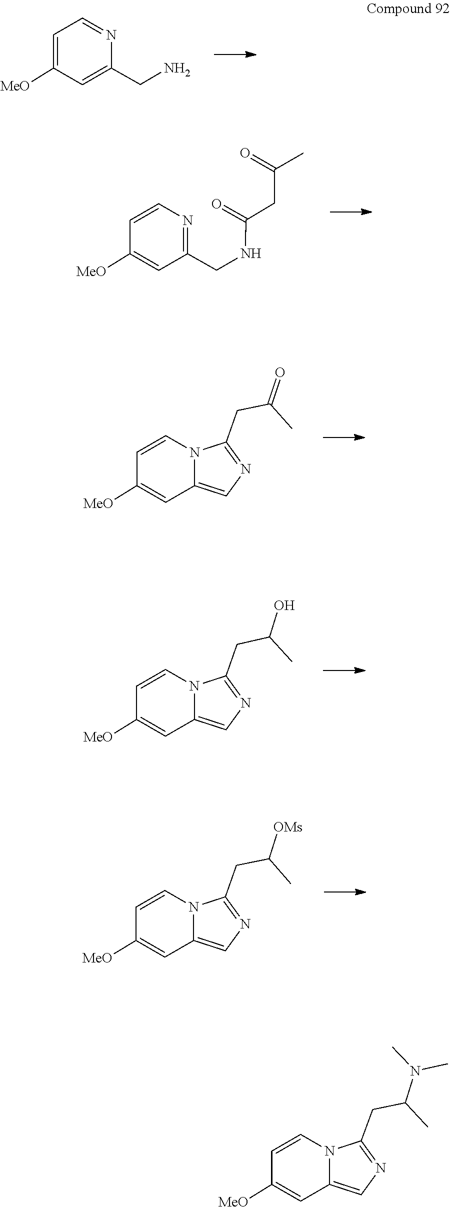 [SMILES: COc1ccnc(CN)c1 ; COc1ccnc(CNC(=O)CC(C)=O)c1 ; COc1ccn2c(CC(C)=O)ncc2c1 ; COc1ccn2c(CC(C)O)ncc2c1 ; COc1ccn2c(CC(C)OS(C)(=O)=O)ncc2c1 ; COc1ccn2c(CC(C)N(C)C)ncc2c1]

### Step 1: Preparation of N-[(4-Methoxy-2-pyridyl)methyl]-3-oxo-butanamide

To a microwave vial charged with (4-methoxy-2-pyridyl)methanamine (250 mg, 1.81 mmol) in toluene (1.5 mL) was added 2,2,6-trimethyl-1,3-dioxin-4-one (257 mg, 1.81 mmol) in one portion. The mixture was heated to 125° C. and stirred for 30 min under microwave irradiation in a Biotage Initiator+ microwave. The mixture was concentrated in vacuo, combined with a previous batch of crude product and purified by column chromatography on silica gel (eluent 1-10% MeOH (containing 2% “880” ammonia) in DCM to afford the title compound (347 mg, 71% over 2 batches) as a viscous oil. Retention time 0.318 min; m/z=[M+H]⁺ calculated for C₁₁H₁₄N₂O₃ 223.1; found 223.2; ¹H NMR (400 MHz, CDCl₃) δ 8.36 (d, J=5.7 Hz, 1H), 7.67 (s, 1H), 6.83-6.67 (m, 2H), 4.54 (d, J=5.1 Hz, 2H), 3.85 (s, 3H), 3.49 (s, 2H), 2.29 (s, 3H).

### Step 2: Preparation of 1-(7-methoxyimidazo[1,5-a]pyridin-3-yl)propan-2-one

To a mixture of N-[(4-methoxy-2-pyridyl)methyl]-3-oxo-butanamide (300 mg, 1.35 mmol) was added phosphoryl trichloride (6.0 mL, 65.5 mmol) at rt. The mixture was heated to 100° C. and stirred for 1 h, then cooled and stirred at rt overnight. The mixture was combined with a previous batch of crude material and concentrated in vacuo. The crude product was purified by column chromatography on silica gel (eluent 1-10% MeOH (containing 2% “880” ammonia) in DCM) to afford the title compound (40 mg, 12% over 2 batches) as a viscous oil. Retention time 0.913 min; m/z=[M+H]⁺ calculated for C₁₁H₁₂N₂O₂ 205.0; found 205.2; ¹H NMR (400 MHz, CDCl₃) δ 7.63 (dt, J=7.6, 0.9 Hz, 1H), 7.17 (d, J=1.0 Hz, 1H), 6.59 (dd, J=2.4, 0.9 Hz, 1H), 6.34 (dd, J=7.7, 2.5 Hz, 1H), 4.09 (s, 2H), 3.80 (s, 3H), 2.21 (s, 3H).

### Step 3: Preparation of (R/S) 1-(7-methoxyimidazo[1,5-a]pyridin-3-yl)propan-2-ol

To a stirred mixture of 1-(7-methoxyimidazo[1,2-a]pyridin-3-yl)propan-2-one (40 mg, 0.2 mmol) in MeOH (1.5 mL), cooled in an ice bath, was added NaBH₄ (8.9 mg, 0.24 mmol) in portions over 1 min. The mixture was warmed to rt and stirred for 3 h, then diluted with EtOAc (30 mL) and washed with conc. sodium bicarbonate solution (3 mL). The organic layer was dried over Na₂SO₄, filtered, and the filtrate was concentrated in vacuo to afford the title compound (38 mg, 94%) as an oil. Retention time 0.888 min; m/z=[M+H]⁺ calculated for C₁₁H₁₄N₂O₂ 207.1; found 207.0; ¹H NMR (400 MHz, CDCl₃) δ 7.61 (d, J=7.5 Hz, 1H), 7.11 (d, J=0.9 Hz, 1H), 6.57 (d, J=2.4 Hz, 1H), 6.32 (dd, J=7.6, 2.4 Hz, 1H), 4.42 (dtd, J=9.2, 6.2, 3.0 Hz, 1H), 3.80 (d, J=2.7 Hz, 3H), 3.03-2.81 (m, 2H), 1.36 (d, J=6.3 Hz, 3H).

### Step 4: Preparation of (R/S) 2-(7-methoxyimidazo[1,5-a]pyridin-3-yl)-1-methyl-ethyl]methanesulfonate

To a mixture of 1-(7-methoxyimidazo[1,5-a]pyridin-3-yl)propan-2-ol (38 mg, 0.18 mmol) in DCM (3 mL) was added Et₃N (64.2 μL, 0.46 mmol). The mixture was cooled to 0° C. then MsCl (21.4 μL, 0.28 mmol) was added dropwise, in the temperature range 0-5° C. The mixture was warmed to rt and stirred overnight, then diluted with DCM (10 mL) and washed with saturated sodium bicarbonate solution (3 mL). The organic layer was concentrated in vacuo to give the title compound (45 mg, 85%), which was used immediately in the next step without further purification. Retention time 1.041 min; m/z=[M+H]⁺ calculated for C₁₂H₁₆N₂O₄S 285.0; found 285.0.

### Step 5: Preparation of (R/S) 1-(7-methoxyimidazo[1,5-a]pyridin-3-yl)-N,N-dimethyl-propan-2-amine

A 0.5-2 mL Biotage microwave vial was charged with [2-(7-methoxyimidazo[1,5-a]pyridin-3-yl)-1-methyl-ethyl]methanesulfonate (45 mg, 0.16 mmol) in DMF (0.2 mL) was added 40% aq. Me₂NH (714 mg, 6.33 mmol). The vial was sealed and the mixture was heated to 65° C. under microwave irradiation and stirred for 16 h in a Biotage Initiator+ microwave. The mixture was directly purified by preparative HPLC (eluting with 10-100% H₂O (with 0.1% ammonia) in MeCN) to afford the title compound (4 mg, 10%) as an oil. Retention time 4.273 min; m/z=[M+H]⁺ calculated for C₁₃H₁₉N₃O 234.1; found 234.2; ¹H NMR (400 MHz, CD₃OD) δ 7.98 (dt, J =7.7, 0.9 Hz, 1H), 7.15-7.02 (m, 1H), 6.74 (dd, J=2.5, 0.8 Hz, 1H), 6.42 (dd, J=7.7, 2.5 Hz, 1H), 3.81 (s, 3H), 3.28-3.08 (m, 2H), 2.94 (dd, J=14.2, 9.2 Hz, 1H), 2.36 (s, 6H), 0.99 (d, J=6.5 Hz, 3H).

### Synthesis of (R/S) 1-(6-fluoroimidazo[1.5a]pyridin-1-yl)propan-2-yl dimethylamine

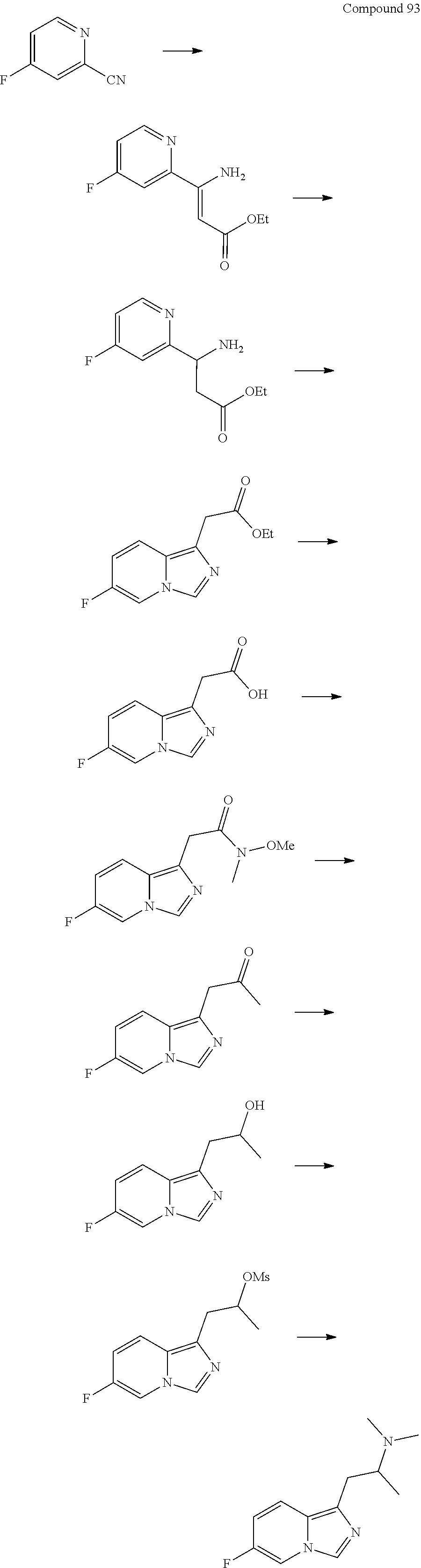

### Step 1: Preparation of ethyl 3-amino-(5-fluoropyridin-2-yl)prop-2-enoate

To a mixture of 5-fluoropyridine-2-carbonitrile (9.11 g, 73.8 mmol), ZnCl₂ (5.08 g, 3.73 mmol), ⁱPr₂NEt (15.3 mL, 89.5 mmol) and ethyl malonate potassium salt (19.0 g, 112.0 mmol) in 1,2 dichloroethane (150 mL) was heated under reflux using a Dean and Stark apparatus for 4.25 h.

The mixture was cooled to rt and DCM (360 mL) and sat NH₄Cl (250 mL) were added. The aqueous and organic layers were separated, and the organic layer was washed with brine (360 mL), dried (MgSO₄), filtered and the filtrate was concentrated in vacuo. The crude product was purified by column chromatography on silica gel (eluent: 0 to 100% EtOAc in hexane) to afford the title compound (8.45 g, 54%) as a solid. ¹H NMR (400 MHz, CDCl₃) δ 8.47 (dd, J=2.9, 0.6 Hz, 1H), 7.83-7.70 (m, 1H), 7.46 (ddd, J=8.9, 7.9, 2.9 Hz, 1H), 5.28 (s, 1H), 4.20 (q, J=7.1 Hz, 2H), 1.31 (t, J=7.1 Hz, 3H).

### Step 2: Preparation of ethyl 3-amino-(5-fluoropyridin-2-yl)propanoate

To a mixture of ethyl 3-amino-(5-fluoropyridin-2-yl)prop-2-enoate (3.0 g, 14.3 mmol), Pd(OH)₂ (20% on carbon, 1.51 g, 2.15 mmol), and AcOH (1.63 mL, 28.6 mmol) in IMS (60 mL) was stirred under an atmosphere of H₂ (initially 30 bar) for 4 h. The mixture was filtered through celite washing with DCM (3×60 mL). The combined filtrate was washed with saturated sodium bicarbonate solution (60 mL), H₂O (60 mL) and brine (60 mL), dried (MgSO₄), filtered and the filtrate was concentrated in vacuo. The residue was purified by column chromatography on silica gel (eluent: 0 to 15% MeOH in DCM) to afford the title compound (5.25 g, 57%) as an oil.

Retention time 1.073 min (10 minute LCMS method); m/z=[M+H]⁺ calculated for C₁₀H₁₃FN₂O₂ 213.2; found 213.2; ¹H NMR (400 MHz, CDCl₃) δ 8.46-8.34 (m, 1H), 7.44-7.32 (m, 2H), 4.44 (dd, J=8.3, 5.2 Hz, 1H), 4.13 (q, J=7.1 Hz, 2H), 2.91-2.63 (m, 2H), 1.23 (t, J=7.1 Hz, 3H).

### Step 3: Preparation of ethyl 2-(6-fluoroimidazo[1,5a]pyridine-1-yl)acetate

To a mixture of ethyl 3-amino-(5-fluoropyridin-2-yl)propanoate (5.3 g, 25.0 mmol) in n-butyl acetate (120 mL) was added formic acid (1.08 mL, 28.7 mmol) followed by dropwise addition of T3P (38.7 mL, 64.9 mmol). The mixture was stirred at rt for 1 h then heated to reflux and stirred for 2.5 h. The mixture was cooled to rt, diluted with EtOAc (250 mL), washed with saturated sodium bicarbonate (2×200 mL) and brine (200 mL), and dried (MgSO₄), filtered and the filtrate was concentrated in vacuo to an oil. The residue was purified by column chromatography on silica gel (eluent: 0 to 1% MeOH in DCM) to afford the title compound (1.49 g, 27%) as an oil. Retention time 1.731 min (10 minute LCMS method); m/z=[M+H]⁺ calculated for C₁₁H₁₁FN₂O₂ 223.0; found 223.0; ¹H NMR (400 MHz, CDCl₃) δ 8.02 (d, J=0.8 Hz, 1H), 7.79 (ddd, J=4.3, 2.0, 0.9 Hz, 1H), 7.44 (ddt, J=10.0, 5.4, 0.8 Hz, 1H), 6.65 (ddd, J=9.8, 7.6, 2.0 Hz, 1H), 4.17 (q, J=7.1 Hz, 2H), 3.91 (s, 2H), 1.26 (t, J=7.1 Hz, 3H).

### Step 4: Preparation of 2-(6-fluoroimidazo[1,5a]pyridine-1-yl)acetic acid

To a mixture of ethyl 2-(6-fluoroimidazo[1,5a]pyridine-1-yl)acetate (1.49 g, 6.7 mmol) and lithium hydroxide (323 mg, 13.4 mmol) in H₂O (5 mL) and IMS (5 mL) was heated to reflux and stirred for 2 h. The mixture was cooled to rt, diluted with H₂O (10 mL), acidified with conc HCl to pH ˜5.5 and extracted with DCM (4×25 mL). The combined organic layers were dried (MgSO₄), filtered and the filtrate was concentrated in vacuo to afford the title compound (447 mg, 34%) as a solid. Retention time 0.601 min (10 minute LCMS method); m/z=[M+H]⁺ calculated for C₉H₇FN₂O₂ 195.0; found 195.0; ¹H NMR (400 MHz, DMSO-d₆) δ 12.22 (s, 1H), 8.44 (ddd, J=5.1, 2.1, 0.9 Hz, 1H), 8.26 (d, J=0.7 Hz, 1H), 7.63 (ddt, J=9.9, 5.5, 0.8 Hz, 1H), 6.81 (ddd, J=10.0, 8.0, 2.1 Hz, 1H), 3.81 (s, 2H).

### Step 5: Preparation of 2-(6-fluoroimidazo[1,5-a]pyridin-1-yl)-N-methoxy-N-methylacetamide

To a mixture of 2-(6-fluoroimidazo[1,5a]pyridine-1-yl)acetic acid (100 mg, 0.51 mmol), 3-(ethyliminomethyleneamino)-N,N-dimethyl-propan-1-amine; hydrochloride (138 mg, 0.72 mmol) and 4-dimethylaminopyridine (97.5 mg, 0.80 mmol) in DMF (1 mL) was stirred for 30 min. N-methoxymethanamine hydrochloride (78 mg, 0.80 mmol) was added and the mixture was stirred at rt for 22 h, then diluted with H₂O (12 mL) and extracted with Et₂O (4×10 mL) and EtOAc (2 ×10 mL). The combined organic layers were dried (MgSO₄), filtered and the filtrate was concentrated in vacuo. The residue was purified by column chromatography on silica gel (eluent: 0 to 20% MeOH in DCM) to afford the title compound (57 mg. 47%) as an oil. Retention time 1.250 min (10 minute LCMS method); m/z=[M+H]⁺ calculated for C₁₁H₁₂FN₃O₂ 238.0; found 238.0; ¹H NMR (400 MHz, CDCl₃) δ 8.01 (d, J=0.7 Hz, 1H), 7.77 (ddd, J=4.3, 2.0, 0.9 Hz, 1H), 7.56 (dd, J=9.9, 5.4 Hz, 1H), 6.62 (ddd, J=9.8, 7.7, 2.0 Hz, 1H), 4.07 (s, 2H), 3.75 (s, 3H), 3.21 (s, 3H).

### Step 6: Preparation of 1-(6-fluoroimidazo[1,5a]pyridine-1-yl)propan-2-one

To a mixture of 2-(6-fluoroimidazo[1,5-a]pyridin-1-yl)-N-methoxy-N-methylacetamide (707 mg, 2.98 mmol) in THF (10 mL) at −78° C. under an atmosphere of N₂ was added MeMgBr (3M in ether, 2.0 mL, 6.0 mmol). The mixture was stirred at −78° C. for 4 h then allowed to warm to rt and stirred overnight. The mixture was quenched with sat NH₄Cl (50 mL) and extracted with EtOAc (3×30 mL). The combined organic layers were washed with brine (30 mL), dried (MgSO₄), filtered and the filtrate was concentrated in vacuo. The residue was purified by column chromatography on silica gel (eluent: 0 to 5% MeOH in DCM) to afford the title compound (104 mg, 18%) as an oil. Retention time 0.831 min (10 minute LCMS method); m/z=[M+H]⁺ calculated for C₁₀H₉FN₂O 193.0; found 193.0; ¹H NMR (400 MHz, CDCl₃) δ 8.05 (d, J=0.7 Hz, 1H), 7.80 (ddd, J=4.2, 2.0, 1.0 Hz, 1H), 7.36 (ddt, J=9.9, 5.3, 0.9 Hz, 1H), 6.65 (ddd, J=9.8, 7.6, 2.0 Hz, 1H), 3.96 (s, 2H), 2.22 (s, 3H).

### Step 7: Preparation of (R/S) 1-(6-fluoroimidazo[1,5a]pyridin-1-yl)propan-2-ol

To a mixture of 1-(6-fluoroimidazo[1,5a]pyridine-1-yl)propan-2-one (104 mg, 0.54 mmol) in MeOH (3 mL) at 0° C. was added NaBH₄ (21 mg, 0.54 mmol). The mixture was stirred at 0° C. for 30 min then warmed to rt and stirred for 30 mins, then quenched with the addition of H₂O (20 mL) and extracted with DCM (3×12 mL). The combined organic layers were dried (MgSO₄), filtered and the filtrate was concentrated in vacuo to afford the title compound (70 mg, 67%) as a solid. Retention time 0.590 min (10 minute LCMS method); m/z=[M+H]⁺ calculated for C₁₀H₁₁FN₂O 195.0; found 195.0; ¹H NMR (400 MHz, CDCl₃) δ 8.02 (d, J=0.8 Hz, 1H), 7.78 (ddd, J=4.3, 2.0, 0.9 Hz, 1H), 7.37 (ddt, J=9.9, 5.3, 0.8 Hz, 1H), 6.60 (ddd, J=9.7, 7.6, 2.0 Hz, 1H), 4.27-4.15 (m, 1H), 3.01 (dd, J=14.8, 3.4 Hz, 1H), 2.86 (dd, J=14.9, 8.5 Hz, 1H), 1.29 (d, J=6.2 Hz, 3H).

### Step 8: Preparation of (R/S) 1-(6-fluoroimidazo[1,5a]pyridine-1-yl)propan-2-yl methanesulfonate

To a mixture of 1-(6-fluoroimidazo[1,5a]pyridin-1-yl)propan-2-ol (70 mg, 0.36 mmol) and Et₃N (63 μL, 0.45 mmol) in DCM (4 mL) was added MsCl (31 μL, 0.4 mmol). The mixture was stirred at rt for 2 h, then additional Et₃N (30 μL, 0.21 mmol) and MsCl (15 μL, 0.2 mmol) were added, and the mixture was stirred at rt for 6 h. Additional Et₃N (33 μL, 0.23 mmol) and MsCl (16 μL, 0.2 mmol) were added and stirring was continued at rt overnight. The mixture was quenched with sat. NaHCO₃ solution (10 mL) and extracted with DCM (2×8 mL). The combined organic layers were dried (MgSO₄), filtered and the filtrate was concentrated in vacuo to afford the title compound (82 mg, 83%) as a viscous oil. Retention time 1.515 min (10 minute LCMS method); m/z=[M+H]⁺ calculated for C₁₁H₁₃FN₂O₃S 273.0; found 273.0.

### Step 9: Preparation of (R/S) 1-(6-fluoroimidazo[1.5a]pyridin-1-yl)propan-2-yl dimethylamine

A 2.5 mL Biotage microwave vial was charged with 1-(6-fluoroimidazo[1,5a]pyridine-1-yl)propan-2-yl methanesulfonate (82 mg, 0.30 mmol) in DMF (0.4 mL) and 40% aq Me₂NH (40%, 1.53 mL, 12.0 mmol) was added. The vial was sealed and the mixture was heated to 65° C. under microwave irradiation and stirred for 16 h in a Biotage Initiator+ microwave. The mixture was purified by preparative HPLC (basic mobile phase) to afford the title compound (21 mg, 32%) as an oil. Retention time 0.761 min (10 minute LCMS method); m/z=[M+H]⁺ calculated for C₁₂H₁₆FN₃ 222.1; found 222.2; ¹H NMR (400 MHz, CD₃OD) δ 8.32-8.13 (m, 2H), 7.68-7.50 (m, 1H), 6.74 (ddd, J=10.0, 7.9, 2.1 Hz, 1H), 3.13 (dd, J=14, 4 Hz, 1H), 3.02 (q, J=2.0 Hz, 1H), 2.78 (dd, J=13.8, 9.8 Hz, 1H), 2.38 (s, 6H), 0.97 (d, J=6.6 Hz, 3H).

### Synthesis of (R/S) 1-(6-methoxyimidazo[1.5a]pyridin-1-yl)propan-2-yl dimethylamine

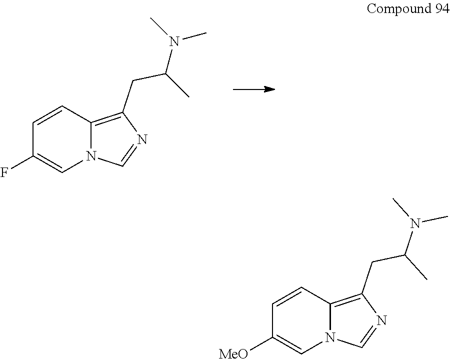 [SMILES: CC(Cc1ncn2cc(F)ccc12)N(C)C ; COc1ccc2c(CC(C)N(C)C)ncn2c1]

To a mixture of 1-(6-fluoroimidazo[1.5a]pyridin-1-yl)propan-2-yl dimethylamine (3.83 mg, 0.017 mmol) and NaOMe (2.2 mg, 0.041 mmol) in MeOH (0.5 mL) was stirred at 60° C. (external) for 40 h, then heated in a Biotage Initiator+ microwave at 100° C. for 90 min, then at 120° C. for 1 h, and 130° C. for 3 h. 15-Crown-5 (1 drop) was added and the mixture was then heated in a Biotage Initiator+ microwave at 130° C. and stirred for 3 h, then at 145° C. for 12 h. LCMS shows the mass of the title compound, as well as the starting material. m/z=[M+H]⁺ calculated for C₁₃H₁₉N₃O 234.1; found 234.2.Synthesis of (R/S) (1-(7-methoxy-[1,2,4]triazolo[4,3a]pyridine-3-yl)proan-2-yl)dimethylamine

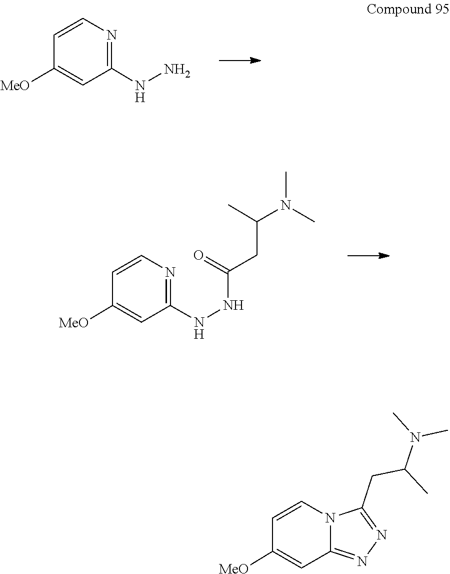 [SMILES: COc1ccnc(NN)c1 ; COc1ccnc(NNC(=O)CC(C)N(C)C)c1 ; COc1ccn2c(CC(C)N(C)C)nnc2c1]

### Step 1: Preparation of (R/S) 3-(dimethylamino)-N′-(4-methoxypryridin-2-yl)butanehydrazide

To a mixture of 3-(dimethylamino)butanoic acid (100 mg, 0.76 mmol) in DCM (2 mL) was added oxalyl chloride (72 μL, 0.85 mmol) and the mixture was stirred at rt for 1 h. A mixture of 2-hydazinyl-4-methoxypyridine dihydrochloride (145 mg, 0.68 mmol) in DMF (4 mL) and K₂CO₃ (230 mg, 2.05 mmol) were added and the mixture was stirred at rt for 4 h. The crude mixture was purified by column chromatography on silica gel (eluent: 0 to 100% (20% MeOH+0.5% NH₃(aq)) in DCM) to afford the title compound (45 mg, 26%) as an oil. Retention time 0.26 min; m/z=[M+H]⁺ calculated for C₁₂H₂₀N₄O₂ 253.3; found 253.0; ¹H NMR (400 MHz, DMSO-d₆) δ 9.74 (br. s, 2H), 7.76 (d, J=6.8 Hz, 1H), 6.46 (dd, J=7.2, 2.4 Hz, 1H), 6.41 (d, J=2.4 Hz, 1H), 3.59 (m, 1H), 2.99 (dd, J=16.4, 3.2 Hz, 2H) 3.86 (s, 3H), 2.65 (s, 6H), 1.27 (d, J=6.4 Hz, 3H).Step 2: Preparation of (R/S) (1-(7-methoxy-[1,2,4]triazolo[4,3a]pyridine-3-yl)proan-2-yl)dimethylamine

A mixture of 3-(dimethylamino)-N′-(4-methoxypryridin-2-yl)butanehydrazide (113 mg, 0.45 mmol) and PCl₅ (140 mg, 0.67 mmol) in DCM (15 mL) was stirred at 40° C. for 23 h. The reaction was quenched with MeOH (0.5 mL) and purified by column chromatography on silica gel (eluent: 0 to 100% (20% MeOH+0.5% NH₃(aq) in DCM) in DCM) to afford the title compound (27 mg, 25%) as a solid. Retention time 0.309 min; m/z=[M+H]⁺ calculated for C₁₂H₁₈N₄O 235.2; found 235.2.

### Prophetic synthesis of (R/S) 1-(7-methoxyindolizin-3-yl)-N,N-dimethylpropan-2-amine

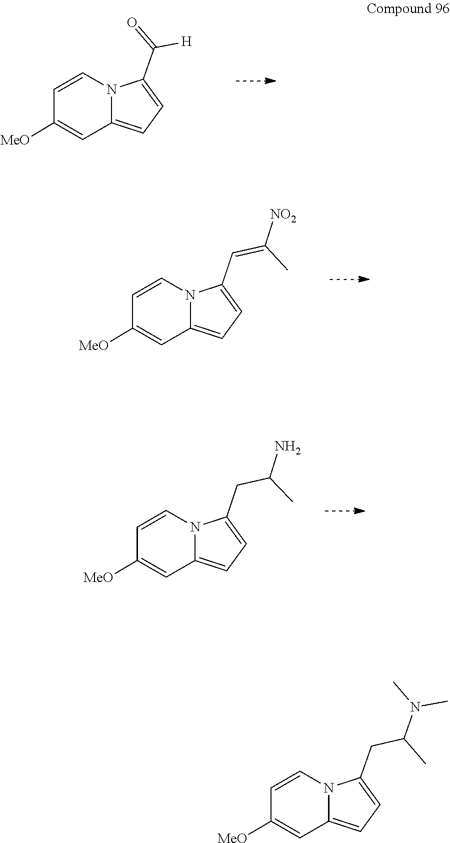 [SMILES: COc1ccn2c(C=O)ccc2c1 ; COc1ccn2c(/C=C(\C)[N+](=O)[O-])ccc2c1 ; COc1ccn2c(CC(C)N)ccc2c1 ; COc1ccn2c(CC(C)N(C)C)ccc2c1]

The title compound can be synthesized from the known aldehyde 7-methoxyindolizine-3-carbaldehyde (CAS No: 1889911-42-5) using the chemistry outlined in the above scheme.

### Synthesis of 1-[(2R)-2-(dimethylamino)propyl]indole-5-carbonitrile

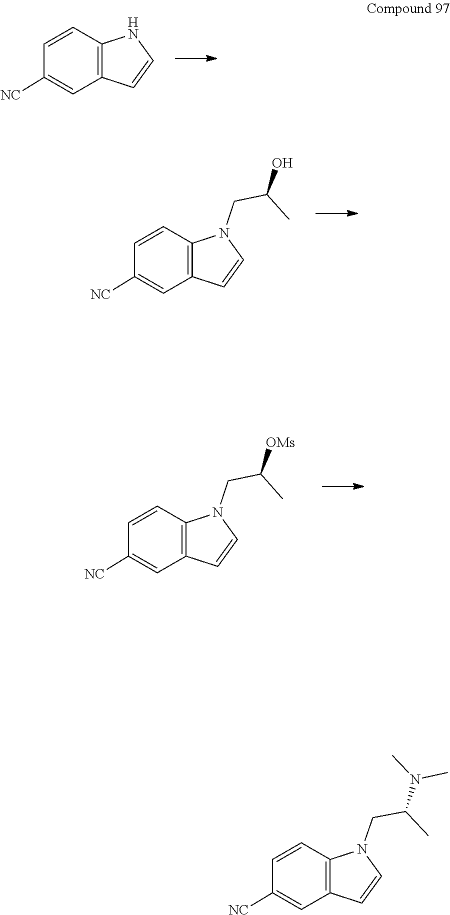 [SMILES: N#Cc1ccc2[nH]ccc2c1 ; C[C@H](O)Cn1ccc2cc(C#N)ccc21 ; C[C@@H](Cn1ccc2cc(C#N)ccc21)OS(C)(=O)=O ; C[C@H](Cn1ccc2cc(C#N)ccc21)N(C)C]

### Step 1: Preparation of 1-[(2S)-2-hydroxypropyl]indole-5-carbonitrile

To a mixture of 1H-indole-5-carbonitrile (3.00 g, 21.1 mmol) in DMF (30 mL) at 0° C. was added NaH, 60% in mineral oil (0.97 g, 25.3 mmol) over 20 min. The mixture was stirred for 30 min, giving a fine suspension, then (S)-propylene oxide (1.23 g, 21.1 mmol) was added fast dropwise. The grey mixture was allowed to warm to rt and then heated to 20° C. for 5 h, becoming a rarer suspension. A further portion of (S)-propylene oxide (0.62 g, 10.7 mmol) and stirring was continued at 20° C. for 96 h. The mixture was quenched with dropwise addition of H₂O (75 mL) with ice-water bath cooling and extracted into Et₂O (5×25 mL). The combined organic layers were washed with 5% LiCl aq (3×80 mL), dried over MgSO₄, filtered and concentrated in vacuo. The residue was dissolved in MeCN (100 mL), washed with hexane (2×20 mL) and concentrated in vacuo to an oil that solidified on standing. The crude product was purified by chromatography on silica gel (eluent: 0% to 100% EtOAc in hexane) to afford the title compound (1.45 g, 34%) as a solid. Retention time 1.46 min; m/z=[M+H]⁺ calculated for C₁₂H₁₂N₂O 201.2; found 201.0; ¹H NMR (400 MHz, DMSO-d₆) δ 8.06 (d, J=0.9 Hz, 1H), 7.69 (d, J=8.7 Hz, 1H), 7.54 (d, J=3.1 Hz, 1H), 7.45 (dd, J=8.7, 1.6 Hz, 1H), 6.58 (dd, J=3.2, 0.9 Hz, 1H), 4.86 (d, J=4.9 Hz, 1H), 4.23-4.05 (m, 2H), 3.97 (ddd, J=11.3, 6.8, 4.6 Hz, 1H), 1.06 (d, J=7.4 Hz, 3H).

### Step 2: Preparation of [(1S)-2-(5-cyanoindol-1-yl)-1-methyl-ethyl]methanesulfonate

To a mixture of 1-[(2S)-2-hydroxypropyl]indole-5-carbonitrile (1.42 g, 7.09 mmol) in DCM (10 mL) was added Et₃N (1.98 mL, 14.2 mmol). The mixture was cooled to 0° C. in an ice-water bath before adding MsCl (0.823 mL, 10.6 mmol) dropwise over 5 min, keeping the temperature in the range 0-5° C. The resulting mixture was warmed to rt and stirred for 30 min. The mixture was cooled in an ice-water bath, quenched by dropwise addition of H₂O (10 mL) and extracted with DCM (3×5 mL). The combined organic layers were dried over MgSO₄, filtered, and the filtrate was concentrated in vacuo. The crude product was triturated in 10% Et₂O in hexane and air dried to afford the title compound (2.0 g, 93%) as a solid. Retention time 1.586 min; m/z=[M+H]⁺ calculated for C₁₃H₁₄N₂₀₃S 279.3; found 279.0; ¹H NMR (400 MHz, DMSO-d₆) δ 8.09 (dd, J=1.7, 0.7 Hz, 1H), 7.76 (d, J=8.7 Hz, 1H), 7.59 (d, J=3.2 Hz, 1H), 7.52 (dd, J=8.6, 1.6 Hz, 1H), 6.65 (dd, J=3.3, 0.9 Hz, 1H), 5.03 (pd, J=6.3, 4.8 Hz, 1H), 4.57-4.44 (m, 2H), 3.27 (s, 1H), 2.67 (s, 3H), 1.36 (d, J=6.3 Hz, 3H).

### Step 3: Preparation of 1-[(2R)-2-(dimethylamino)propyl]indole-5-carbonitrile

To a mixture of [(1S)-2-(5-cyanoindol-1-yl)-1-methyl-ethyl]methanesulfonate (1.00 g, 3.59 mmol) in 40% aqueous dimethylamine (16 mL) was added DMF (5 mL). The mixture was heated to 65° C. and stirred for 16 h in a sealed vessel. The mixture was cooled then diluted with H₂O (45 mL), and extracted with diethyl ether (5×15 mL). The combined organic layers were washed with 5% aq LiCl (40 mL), dried over MgSO₄, filtered, and the filtrate was concentrated in vacuo. The crude product was purified by preparative HPLC (eluent: 15-24% MeCN in 0.1% aq formic acid gradient) to afford the title compound as (0.32 g, 39%) as an oil. Retention time 1.071 min; m/z=[M+H]⁺ calculated for C₁₄H₁₇N₃ 228.3; found 228.2; ¹H NMR (400 MHz, DMSO-d₆) δ 8.06 (dd, J=1.6, 0.7 Hz, 1H), 7.68 (dt, J=8.6, 0.8 Hz, 1H), 7.56 (d, J=3.2 Hz, 1H), 7.46 (dd, J=8.6, 1.6 Hz, 1H), 6.58 (dd, J=3.2, 0.9 Hz, 1H), 4.28 (dd, J=14.3, 7.4 Hz, 1H), 4.10 (dd, J=14.3, 6.9 Hz, 1H), 3.03 (dt, J=7.3, 6.6 Hz, 1H), 2.20 (s, 6H), 0.84 (d, J=6.6 Hz, 3H).

Additional reagents and starting materials useful to make the presently disclosed compounds are known to those of ordinary skill in the art.

### Example 2: Evaluation of Metabolic Stability in Human Liver Microsomes

The test compound at 1.0 μM in singlet, or positive controls including Testosterone (CYP3A4 substrate), Propafenone (CYP2D6 substrate) or Diclofenac (CYP2C9 substrate) was incubated with the liver microsomes (Corning, Xenotech, or other credible vendor, pooled from multiple donors) at 0.5 mg/mL, respectively. The mixture was warmed up at 37° C. for 10 minutes and the reactions were initiated by the addition of a NADPH regenerating system (˜1.0 mM). The test compound incubated with the liver microsomes at 37° C. without the NADPH regenerating system served as the negative control reaction.

The reaction samples were removed at multiple time points (such as 0, 5, 15, 30, 45 and 60 minutes) and the sample without NADPH (NCF) was removed at 60 minutes. All the samples were immediately mixed with cold acetonitrile containing internal standard (IS) to stop the reaction.

Samples were analyzed by LC/MS/MS and the disappearance of test compound were assessed base on peak area ratios of analyte/IS (no standard curve).
The microsomal intrinsic clearance and T_{1/2} values were calculated using the following equation:

$${C}_{t} = {C}_{0} {e}^{- {k}_{e} t}$$

$${T}_{1 / 2} = \frac{{L}_{n}}{- {k}_{e}} = \frac{0.693}{- {k}_{e}}$$

The microsomal intrinsic clearance and T_{1/2} values were calculated using the following equation:

$${CL}_{int ( mic )} = \frac{0.693}{in vitro T 1 / 2} \times \frac{1}{mg / mL microsomal protein in reaction mixture}$$

The mg microsomal protein/g liver weight was 45 for 5 species

The liver weight values used 40 g/kg, 30 g/kg, 32 g/kg, 20 g/kg and 88 g/kg for rat, monkey, dog, human and mouse, respectively.

The liver clearance were calculated using CL_{int(mic)} with,

$$CLint ( liver ) = CLint ( mic ) \times \frac{mg microsomes}{g liver} \times \frac{g liver}{kg body weight}$$

In these experiments the comparator compound was a suitable reference standard. In one embodiment the comparator compound was (R)-1-(5-methoxy-1H-indol-1-yl)-N,N′-dimethylpropan-2-amine.

TABLE 2
Metabolic stability in human liver microsomes of representative compounds

| | Compound | T_{1/2} | CL_{int(mic)} | CLᵢₙₜ₍ₗᵢᵥₑᵣ₎ |
|---|---|---|---|---|
| Compound ID | Number | (min) | (μL/min/mg) | (mL/min/kg) |
| (2R)-1,1,1,2,3,3-hexadeuterio-3-(5- | 5 | 95.4** | 15.0 * | 13.5** |
| methoxyindol-1-yl)-N,N-dimethyl- | | | | |
| propan-2-amine | | | | |
| (2R)-1,1,1,2,3,3-hexadeuterio-3-(5- | 6 | 109.1* | 12.7* | 11.4* |
| methoxyindol-1-yl)-N,N- | | | | |
| bis(trideuteriomethyl)propan-2-amine | | | | |
| (2R)-1,1,1,2,3,3-hexadeuterio-N,N- | 151 | 120.3* | 11.5* | 10.4* |
| dimethyl-3-[5- | | | | |
| (trideuteriomethoxy)indol-1-yl]propan- | | | | |
| 2-amine | | | | |
| (2R)-1,1,1,2,3,3-hexadeuterio-3-[5- | 152 | 114.3** | 12.4** | 11.1** |
| (trideuteriomethoxy)indol-1-yl]-N,N- | | | | |
| bis(trideuteriomethyl) propan-2-amine | | | | |
| (R)-1-(5-methoxy-1H-indol-1-yl)-N,N- | Reference | 85.0*** | 16.4*** | 14.8*** |
| dimethylpropan-2-amine | Compound | | | |

*Results from 1 experiment;
**Average of 2 experiments;
***Average of 3 experiments.
R²: correlation coefficient of the linear regression for the determination of kinetic constant Values with R² < 0.6 were excluded from the calculations in the table above.
CL_{int(mic)}: intrinsic clearance
CL_{int(mic)} = 0.693/T_{1/2}/mg microsome protein per mL
CLᵢₙₜ₍ₗᵢᵥₑᵣ₎ = CL_{int(mic)} * mg microsomal protein/g liver weight * g liver weight/kg body weight
T1/2: half life

Based on the results in Table 2, (2R)-1,1,1,2,3,3-hexadeuterio-N,N-dimethyl-3-[5-(trideuteriomethoxy)indol-1-yl]propan-2-amine (Compound 151) demonstrated the most significant difference in half-life and intrinsic clearance ratio compared with (R)-1-(5-methoxy-1H-indol-1-yl)-N,N-dimethylpropan-2-amine. Additionally, Compounds 5, 6, and 152 demonstrated significant and beneficial differences in half-life and intrinsic clearance compared to reference compound (R)-1-(5-methoxy-1H-indol-1-yl)-N,N-dimethylpropan-2-amine.

### Example 3: Oral Bioavailability in Rats—Pharmacokinetics of Test Articles Following a Single Intravenous or Oral Administration in Rats

A pharmacokinetic (PK) study is performed in three male Sprague-Dawley (SD) rats following intravenous (IV) and oral (PO) administration of comparator, or test compound of the invention, at 1 mg/kg (IV) and 10 (PO) mg/kg. Test compounds, or comparator, are measured in plasma.

A detailed description of the in vivo methods:

### Rat Strain

Rats used in these studies are supplied by Charles River (Margate UK) and are specific pathogen free. The strain of rats is Sprague Dawley. Male rats are 175-225g on receipt and are allowed to acclimatize for 5-7 days.

### Animal Housing

Rats are group housed in sterilised individual ventilated cages that expose the animals at all times to HEPA filtered sterile air. Animals will have free access to food and water (sterile) and will have sterile aspen chip bedding (at least once weekly). The room temperature is 22° C. +/−1° C., with a relative humidity of 60% and maximum background noise of 56 dB. Rats are exposed to 12 hour light/dark cycles.

### Treatment

Test article is diluted 10% v/v DMSO, 40% v/v PEG-400, 50% v/v Water. The test articles are administered in a dose volume of 2 mL/kg for intravenous (IV) and 5 mL/kg (PO) for oral routes of administration.

### Example 4:Single IV/PO dose pharmacokinetics study in rats

Each test article is administered as a single IV bolus (via a lateral tail-vein) or a single oral gavage in cohorts of 3 rats per route. Following dose administrations, a 100 μL whole blood sample (EDTA) is collected via the tail-vein at time-points described in Table 3. The blood is centrifuged to separate plasma. Approximately 40 μL of plasma is dispensed per time-point, per rat, in a 96 well plate and frozen until analysis. Bioanalysis is carried out on plasma samples.

TABLE 3
Single IV and oral dose pharmacokinetics profile of test articles in rat plasma

| | | | | | No. |
|---|---|---|---|---|---|
| | | | Dose | Blood sample | of |
| Group | Test article | Route | (mg/kg) | collection (post dose) | rats |
| 1 | Comparator | IV | 1 | 5 min, 15 min, 30 min, 1 h, | 3 |
| | | | | 2 h, 4 h, 7 h, 24 h | |
| 2 | Comparator | PO | 10 | 15 min, 30 min, 45 min, 1 h, | 3 |
| | | | | 2 h, 4 h, 7 h, 24 h | |
| 3 | Test Article | IV | 1 | 5 min, 15 min, 30 min, 1 h, | 3 |
| | | | | 2 h, 4 h, 7 h, 24 h | |
| 4 | Test Article | PO | 10 | 15 min, 30 min, 45 min, 1 h, | 3 |
| | | | | 2 h, 4 h, 7 h, 24 h | |

Dose formulation Samples

Dose formulation samples are diluted in two steps with 50:50 (v/v) methanol/water to an appropriate concentration, then diluted 10:90 (v/v) with control matrix to match to the calibration standard in plasma.

### Sample Extraction Procedure

Calibration and QC standards, incurred samples, blank matrix and dose formulation samples are extracted by protein precipitation, via the addition of a bespoke acetonitrile (ACN)-based Internal Standard (IS) solution, containing several compounds and including Metoprolol and Rosuvastatin, both of which are monitored for during analysis. Following centrifugation, a 40 μL aliquot of supernatant is diluted by the addition of 80 μL water. The prepared sample extracts are analysed by LC-MS/MS.

### Example of Bioanalytical Method and Assay Information Document

1 According to the plate layout, aliquot to wells in 0.8 mL 96-well plate (Abgene). 30 μL for Calibration, QC standards, blanks and dose formulation check.
2 Prepare Calibration and QC standards according to the assay information. Dilute dose formulation according to the assay information. Aliquot incurred samples according to the plate layout & assay information.
3 Add 90 μL of ACN internal standard and vortex mix for 5 minutes at 850 rpm
4 Centrifuge at nominally 4000 rpm for 10 minutes
6 Transfer 40 μL of supernatant into a new 0.8 mL Abgene plate.
6 Add 80 μL of water to all transferred supernatant.
7 Vortex mix for 30 seconds at 1400 rpm
8 Analyse immediately by LC-MS/MS or store at +4° C. until analysis.

To determine if the presently disclosed compounds exhibit differences in psychoplastogenic potency, concentration-response experiments are performed. Disclosed compounds exhibit maximum efficacies and have similar potencies as DMT and known analogs thereof. The present compounds also are capable of increasing dendritic arbor complexity at concentrations such as 100 nM, 10 nM or even 1 nM. These compounds exhibit comparable efficacies and potencies to ketamine, further emphasizing their potential as antidepressants. Particular embodiments exhibit low hallucinogenic potential in both drug-discrimination and head-twitch response (HTR) assays.

DMT and other psychedelic compounds promote increased dendritic arbor complexity, dendritic spine density, and synaptogenesis through a 5-HT_{2A}-dependent process. Pretreating cortical cultures with a 5-HT_{2A} antagonist blocked the ability of 5-MeO-DMT to increase dendritic growth. Importantly, the psychoplastogenic effects of the present compounds also are blocked under these conditions, implicating the 5-HT_{2A} receptor in their mechanism of action.

To determine hallucinogenic potential, the presently disclosed compounds are administered to mice and HTR is evaluated. Hallucinogenic compounds, such as 5-MeO-DMT produce a robust, dose-dependent HTR. However, we expect that at least some of the presently disclosed compounds are significantly less potent. Further, certain exemplary potent plasticity-promoting compounds disclosed herein do not produce any HTR, demonstrating that hallucinogenic potential and psychoplastogenicity can be decoupled.

Hallucinogens (e.g., LSD and 5-MeO-DMT) activate a 5HT_{2A} sensor assay in agonist mode, but their non-hallucinogenic congeners (lisuride (LIS) and 6-MeO-DMT) do not. Moreover, compounds, such as, for example, 5-MeO-DMT, LSD, DMT, DOI, which are hallucinogenic in animals (e.g., humans), activate the 5HT_{2A} sensor assay in agonist mode, whereas compounds, such as, for example, 6-MeO-DMT, LIS, 6-F-DET, L-MDMA, R-MDMA, Ketanserin, BOL148, which are non-hallucinogenic in animals (e.g., humans), do not activate the 5HT_{2A} sensor assay in agonist mode. In some embodiments, hallucinogenic potential of a compound of the present invention is determined in vitro. In some embodiments, hallucinogenic potential of a compound of the present invention is determined using a 5HT_{2A} sensor assay. In some embodiments, the 5HT_{2A} sensor assay is in an agonist mode or an antagonist mode. In some embodiments, the 5HT_{2A} sensor assay is in an agonist mode. In some embodiments, a compound of the present invention that does not activate the sensor in agonist mode has non-hallucinogenic potential. In some embodiments, a compound of the present invention that does not activate the sensor in agonist mode is a non-hallucinogenic compound.

### Example 5: Agonist and Antagonist Profiles at Select 5-hydroxytryptamine (5-HT; Serotonin) Receptors

Protocol—in vitro testing at select 5-hydroxytryptamine (5-HT; serotonin) receptors was conducted.

### Assay Design: Calcium Mobilization

### Cell Handling

1. Cell lines were expanded from freezer stocks according to standard procedures.
2. Cells were seeded in a total volume of 20 μL into black-walled, clear-bottom, Poly-D-lysine coated 384-well microplates and incubated at 37° C. for the appropriate time prior to testing.

### Dye Loading

1. Assays were performed in 1× Dye Loading Buffer consisting of 1× Dye, 1× Additive A and 2.5 mM Probenecid in HBSS/20 mM Hepes. Probenicid was prepared fresh.
2. Cells were loaded with dye prior to testing. Media was aspirated from cells and replaced with 20 μL Dye Loading Buffer.
3. Cells were incubated for 30-60 minutes at 37° C.

### Agonist Format

1. For agonist determination, cells were incubated with sample to induce response.
2. After dye loading, cells were removed from the incubator and 10 μL HBSS/20 mM Hepes was added. 3× vehicle was included in the buffer when performing agonist dose curves to define the EC80 for subsequent antagonist assays. Cells were incubated for 30 minutes at room temperature in the dark to equilibrate plate temperature.
3. Intermediate dilution of sample stocks was performed to generate 4× sample in assay buffer.
4. Compound agonist activity was measured on a FLIPR Tetra (MDS). Calcium mobilization was monitored for 2 minutes and 10 μL 4× sample in HBSS/20 mM Hepes was added to the cells 5 seconds into the assay.

### Allosteric Modulation Format

1. For allosteric determination, cells were pre-incubated with sample followed by agonist induction at the EC20 concentration.
2. Intermediate dilution of sample stocks was performed to generate 3× sample in assay buffer.
3. After dye loading, cells were removed from the incubator and 10 μL 3× sample was added. Cells were incubated for 30 minutes at room temperature in the dark to equilibrate plate temperature. Vehicle concentration was 1%.
4. Compound allosteric activity was measured on a FLIPR Tetra (MDS). Calcium mobilization was monitored for 2 minutes and 10 μL of 4× EC20 agonist in HBSS/20 mM Hepes was added to the cells 5 seconds into the assay.

### Antagonist Format

1. For antagonist determination, cells were pre-incubated with sample followed by agonist challenge at the EC80 concentration.
2. Intermediate dilution of sample stocks was performed to generate 3× sample in assay buffer.
3. After dye loading, cells were removed from the incubator and 10 μL 3× sample was added. Cells were incubated for 30 minutes at room temperature in the dark to equilibrate plate temperature. Vehicle concentration was 1%.
4. Compound antagonist activity was measured on a FLIPR Tetra (MDS). Calcium mobilization was monitored for 2 minutes and 10 μL EC80 agonist in HBSS/20 mM Hepes was added to the cells 5 seconds into the assay.

### Data Analysis

1. Compound activity was analyzed using CBIS data analysis suite (ChemInnovation, CA).
2. For agonist mode assays, percentage activity is calculated using the following formula: % Activity=100% ×(mean RFU of test sample−mean RFU of vehicle control)/(mean MAX RFU control ligand−mean RFU of vehicle control).
3. For positive allosteric mode assays, percentage modulation was calculated using the following formula: % Modulation=100% ×((mean RFU of test sample−mean RFU of EC20 control)/(mean RFU of MAX control ligand−mean RFU of EC20 control)).
4. For antagonist and negative allosteric modulation mode assays, percentage inhibition is calculated using the following formula: % Inhibition=100% ×(1−(mean RFU of test sample−mean RFU of vehicle control)/(mean RFU of EC80 control−mean RFU of vehicle control)).

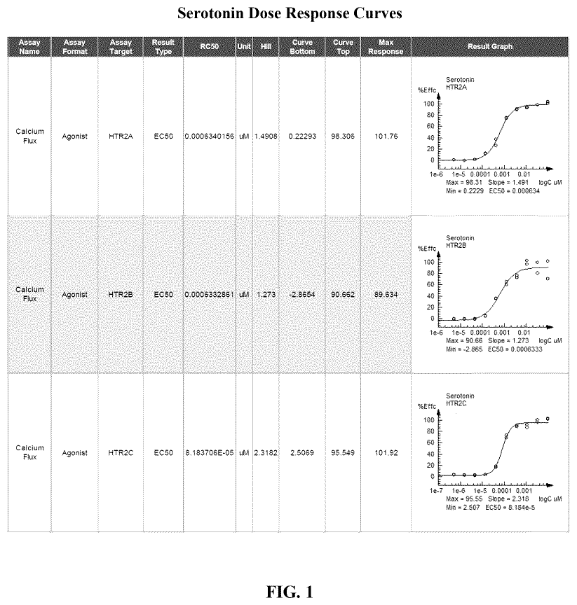

FIG. 1 shows control dose response curves for the selected GPCR Biosensor Assays.

TABLE 4
Activity of compounds against 5-HT2A, 5-HT2B and 5-HT2C receptors.

| | | 5-HT2A | 5-HT2A | 5-HT2B | 5-HT2C |
|---|---|---|---|---|---|
| | | agonist | antagonist | agonist | antagonist |
| Compound | | efficacy | inhibition | efficacy | inhibition |
| Number | Structure & Name | @10 mM | @100 nM | @5 mM | @10 mM |
| — | 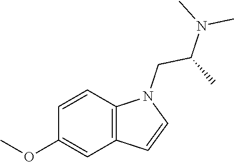 | 1.0% | 3.2% | −2.9% | 35.0% |
| | (R)-1-(5-methoxy-1H-indol-1- | | | | |
| | yl)-N,N-dimethylpropan-2- | | | | |
| | amine(AAZ-A-154) - | | | | |
| | literature compound (Cell | | | | |
| | 2021, 184, 2779-2792.e18) | | | | |
| 5 | 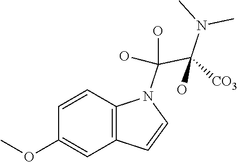 | 2.0% | −2.7% | 3.0% | 38.1% |
| | (R)-1-(5-methoxy-1H-indol-1- | | | | |
| | yl)-N,N-dimethylpropan-d6-2- | | | | |
| | amine | | | | |
| 4 | 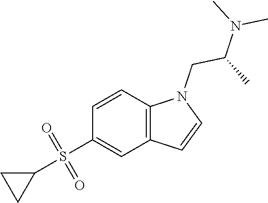 [SMILES: C[C@H](Cn1ccc2cc(S(=O)(=O)C3CC3)ccc21)N(C)C] | −1.5% | −9.1% | −0.6% | 6.2% |
| | ((2R)-1-(5- | | | | |
| | cyclopropylsulfonylindol-l-yl)- | | | | |
| | N,N-dimethyl-propan-2-amine) | | | | |
| 1 | 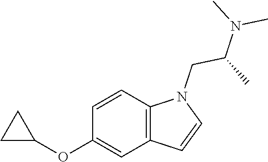 [SMILES: C[C@H](Cn1ccc2cc(OC3CC3)ccc21)N(C)C] | −1.4% | −13.2% | −0.7% | 20.7% |
| | (R)-1-(5-cyclopropoxy-1H- | | | | |
| | indol-1-yl)-N,N- | | | | |
| | dimethylpropan-2-amine | | | | |
| 2 | 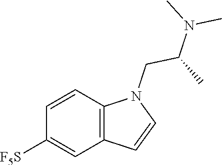 | 4.4% | −0.8% | 6.4% | 76.4% |
| | (2R)-N,N-dimethyl-1-[5- | | | | |
| | (pentafluoro-λ⁶-sulfanyl)indol- | | | | |
| | 1-yl]propan-2-amine | | | | |
| 3 | 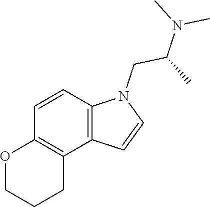 [SMILES: C[C@H](Cn1ccc2c3c(ccc21)OCCC3)N(C)C] | 9.7% | −7.3% | 1.0% | 98.1% |
| | (R)-1-(8,9-dihydropyrano[3,2- | | | | |
| | e]indol-3(7H)-yl)-N,N- | | | | |
| | dimethylpropan-2-amine | | | | |
| 93 | 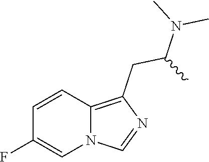 [SMILES: CC(Cc1ncn2cc(F)ccc12)N(C)C] | 1.8% | 5.7% | 2.7% | 97.1% |
| | (R/S)-1-(6-fluoroimidazo[1,5- | | | | |
| | a]pyridin-1-yl)-N,N- | | | | |
| | dimethylpropan-2-amine | | | | |
| 91 | 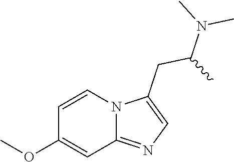 | 0.6% | −3.3% | 1.0% | 6.1% |
| | (R/S)-1-(7- | | | | |
| | methoxyimidazo[1,2- | | | | |
| | a]pyridin-3-yl)-N,N- | | | | |
| | dimethylpropan-2-amine | | | | |
| 92 | 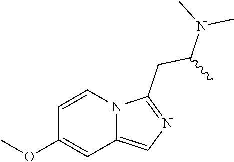 | 0.3% | 1.6% | 2.0% | 7.2% |
| | (R/S)-1-(7- | | | | |
| | methoxyimidazo[1,5- | | | | |
| | a]pyridin-3-yl)-N,N- | | | | |
| | dimethylpropan-2-amine | | | | |

A compound was deemed to have an advantageous property if it was found to be an agonist, or partial agonist, or antagonist, of the 5-HT2A receptor, when screened at a concentration of 10 μM (ten micromolar), whilst also not serving as an agonist of the 5-HT2B receptor (defined as <20% relative efficacy in relation to 5-HT) at a screening concentration of 5 μM (five micromolar). Agonism, or partial agonism, of the 5-HT2A receptor (also known as positive allosteric modulation, or 5-HT2A modulation) is useful for the treatment of neurological and psychiatric disorders. For example, 5-HT2A agonism has been correlated with the promotion of neural plasticity (Ly et al., 2018). Antagonism of the 5HT2A receptor is also useful for the treatment of neurological and psychiatric disorders (Mestre et al., Expert Opinion Investigational Drugs 2013, 22, 411-421). Agonism of the 5-HT2B receptor has been associated with unwanted cardiac valvulopathy side-effects, a form of cardio-toxicity (Rothman et al., Circulation. 2000, 102, 2836-2841; Fitzgerald et al., Molecular Pharmacology 2000, 57, 75-81).

A compound was also deemed to have an advantageous property if it was found to be an antagonist of the 5-HT2C receptor, when screened at a concentration of 10 μM (ten micromolar).

Antagonists of the 5-HT2C receptor are also useful for the treatment of neurological and psychiatric disorders (Kennett et al., Neuropharmacology 1997, 36, 609-620).

Compounds disclosed herein with desirable properties may serve as antagonists of the 5-HT2A receptor, and do not serve as agonists of the 5-HT2B receptor at 5 μM (five micromolar).

Compounds were evaluated as 5-HT2A antagonists at 100 nM (one hundred nanomolar), or may be evaluated at a higher concentration. For research comparative purposes, a literature compound (R)-1-(5-methoxy-1H-indol-1-yl)-N,N-dimethylpropan-2-amine (AAZ-A-154) (Cell 2021, 184, 2779-2792.e18)—was included in the screening assays. Additionally, 5-hydroxytryptamine/serotonin (5-HT) was included in the screening assay as a positive control agonist, and Altanserin HCl was included as a 5-HT2A antagonist positive control. Compounds that were shown to advantageous properties at the 5-HT2A or 5-HT2B receptor as discussed herein include, (R)-1-(8,9-dihydropyrano[3,2-e]indol-3(7H)-yl)-N,N-dimethylpropan-2-amine —compound 3, (R)-1-(5-methoxy-1H-indol-1-yl)-N,N-dimethylpropan-d6-2-amine-compound 5, (R/S)-1-(6-fluoroimidazo[1,5-a]pyridine-1-yl)-N,N-dimethylpropan-2-amine—compound 93, (R/S)-1-(7-methoxyimidazo[1,5-a]pyridin-3-yl)-N,N-dimethylpropan-2-amine—compound 92.

Compounds may also serve as antagonists of the 5-HT2C receptor at 10 μM (ten micromolar). For research comparative purposes, a literature compound—(R)-1-(5-methoxy-1H-indol-1-yl)-N,N-dimethylpropan-2-amine (AAZ-A-154) (Cell 2021, 184, 2779-2792.e18)—was included in the screening assays. Additionally, 5-hydroxytryptamine/serotonin (5-HT) was included in the screening assay as a positive control agonist, and SB242084 was used in the screening assay as a literature 5-HT2C antagonist positive control. Compounds that were shown to have advantageous properties at the 5-HT2C receptor as discussed herein include, but are not limited to: (R)-1-(5-methoxy-1H-indol-1-yl)-N,N-dimethylpropan-d6-2-amine (AAZ-d6)—compound 5, (R)-1-(5-cyclopropoxy-1H-indol-1-yl)-N,N-dimethylpropan-2-amine—compound 1, (2R)—N,N-dimethyl-1-[5-(pentafluoro-λ⁶-sulfanyl)indol-1-yl]propan-2-amine—compound 2, (R)-1-(8,9-dihydropyrano[3,2-e]indol-3(7H)-yl)-N,N-dimethylpropan-2-amine—compound 3, (R/S)-1-(6-fluoroimidazo[1,5-a]pyridin-1-yl)-N,N-dimethylpropan-2-amine—compound 93, and (R/S)-1-(7-methoxyimidazo[1,5-a]pyridine-3-yl)-N,N-dimethylpropan-2-amine—compound 92. Compounds 2, 3 and 93 each achieved greater than 75% antagonism of the 5-HT2C receptor, with compounds 3 and 93 achieving approximately 98% and 97% antagonism, respectively.

### Example 6: Calcium Flux Assay

Calcium Secondary Messenger Pathway: The Calcium No Wash^{PLUS} assay monitors the activation of a GPCR (e.g., 5HT2A) via Gq secondary messenger signaling in a live cell, non-imaging assay format. Calcium mobilization in PathHunter® cell lines or other cell lines stably expressing Gq-coupled GPCRs (e.g., 5HT2A) is monitored using a calcium-sensitive dye that is loaded into cells. GPCR (e.g., 5HT2A) activation by a compound results in the release of calcium from intracellular stores and an increase in dye fluorescence that is measured in real-time. In some embodiments, the ability of a compound of the present invention to modulate 5-HT_{2A} function is determined using a calcium flux assay. In some embodiments, a compound of the present invention activates a calcium flux assay. In some embodiments, the activation of a calcium flux assay indicates that a compound of the present invention modulates 5-HT_{2A} function. In some embodiments, the ability of the compounds of the present invention to modulate 5-HT_{2A} function is assessed from the results of the calcium flux assay.

To perform the calcium flux assay, cell lines are expanded from freezer stocks according to standard procedures. Cells are seeded in a total volume of 20 μL into black-walled, clear-bottom, Poly-D-lysine coated 384-well microplates and incubated at 37° C. for the appropriate time prior to testing. Assays are performed in 1× Dye Loading Buffer consisting of 1× Dye, 1× Additive A and 2.5 mM Probenecid in HBSS/20 mM Hepes. Probenicid is prepared fresh. Cells are loaded with dye prior to testing. Media is aspirated from cells and replaced with 20 μL Dye Loading Buffer. Cells are incubated for 30-60 minutes at 37° C.

For agonist determination, cells are incubated with sample to induce response. After dye loading, cells are removed from the incubator and 10 μL HBSS/20 mM Hepes is added. 3× vehicle is included in the buffer when performing agonist dose curves to define the EC80 for subsequent antagonist assays. Cells are incubated for 30 minutes at room temperature in the dark to equilibrate plate temperature. Intermediate dilution of sample stocks is performed to generate 4× sample in assay buffer. Compound agonist activity is measured on a FLIPR Tetra (MDS). Calcium mobilization is monitored for 2 minutes and 10 μL 4× sample in HBSS/20 mM Hepes is added to the cells 5 seconds into the assay.

Compound activity is analyzed using CBIS data analysis suite (Chemlnnovation, CA). For agonist mode assays, percentage activity is calculated using the following formula:

$$\% Activity = 100 \% \times ( mean RFU of test sample - mean RFU of vehicle control ) / ( mean MAX RFU control ligand - mean RFU of vehicle control ) .$$

Dendritogenesis Assays. Compounds disclosed herein are evaluated for their ability to increase dendritic arbor complexity in cultures of cortical neurons using a phenotypic assay.

Following treatment, neurons are fixed and visualized using an antibody against MAP2—a cytoskeletal protein localized to the somatodendritic compartment of neurons. Sholl analysis is then performed, and the maximum number of crossings (Nₘₐₓ) is used as a quantitative metric of dendritic arbor complexity. For statistical comparisons between specific compounds, the raw Nₘₐₓ values are compared. Percent efficacies are determined by setting the Nₘₐₓ values for the vehicle (DMSO) and positive (ketamine) controls equal to 0% and 100%, respectively.

Animals. For the dendritogenesis experiments, timed pregnant Sprague Dawley rats are obtained. For the head-twitch response assay, male and female C57BL/6J mice are obtained.

Dendritogenesis—Sholl Analysis. Dendritogenesis experiments are performed following a previously published methods with slight modifications. Neurons are plated in 96-well format (200 μL of media per well) at a density of approximately 15,000 cells/well in Neurobasal (Life Technologies) containing 1% penicillin-streptomycin, 10% heat-inactivated fetal bovine serum, and 0.5 mM glutamine. After 24 h, the medium is replaced with Neurobasal containing 1× B27 supplement (Life Technologies), 1% penicillin-streptomycin, 0.5 mM glutamine, and 12.5 μM glutamate. After 3 days in vitro (DIV3), the cells are treated with compounds. All compounds tested in the dendritogenesis assays are treated at 10 μM. Stock solutions of the compounds in DMSO are first diluted 100-fold in Neurobasal before an additional 10-fold dilution into each well (total dilution=1:1000; 0.1% DMSO concentration). Treatments are randomized. After 1 h, the media is removed and replaced with new Neurobasal media containing 1× B27 supplement, 1% penicillin-streptomycin, 0.5 mM glutamine, and 12.5 mM glutamate. The cells are allowed to grow for an additional 71 h. At that time, neurons are fixed by removing 80% of the media and replacing it with a volume of 4% aqueous paraformaldehyde (Alfa Aesar) equal to 50% of the working volume of the well. Then, the cells are incubated at room temperature for 20 min before the fixative is aspirated and each well washed twice with DPBS. Cells are permeabilized using 0.2% Triton X-100 (ThermoFisher) in DPBS for 20 minutes at room temperature without shaking. Plates are blocked with antibody diluting buffer (ADB) containing 2% bovine serum albumin (BSA) in DPBS for 1 h at room temperature. Then, plates are incubated overnight at 4° C. with gentle shaking in ADB containing a chicken anti-MAP2 antibody (1:10,000; EnCor, CPCA-MAP2). The next day, plates are washed three times with DPBS and once with 2% ADB in DPBS. Plates are incubated for 1 h at room temperature in ADB containing an anti-chicken IgG secondary antibody conjugated to Alexa Fluor 488 (Life Technologies, 1:500) and washed five times with DPBS. After the final wash, 100 μL of DPBS is added per well and imaged on an ImageXpress Micro XL High-Content Screening System (Molecular Devices, Sunnyvale, CA) with a 20× objective. Images are analyzed using ImageJ Fiji (version 1.51 W). First, images corresponding to each treatment are sorted into individual folders that are then blinded for data analysis. Plate controls (both positive and negative) are used to ensure that the assay is working properly as well as to visually determine appropriate numerical values for brightness/contrast and thresholding to be applied universally to the remainder of the randomized images. Next, the brightness/contrast settings are applied, and approximately 1-2 individual pyramidal-like neurons per image (i.e., no bipolar neurons) are selected using the rectangular selection tool and saved as separate files. Neurons are selected that do not overlap extensively with other cells or extend far beyond the field of view.

Ketanserin Blocking Experiments. For the ketanserin blocking experiments, a slightly modified method is employed. On DIV 3, neurons are first treated with ketanserin (10 mM) for 1 h followed by a 1 h incubation with drug (1 mM) and ketanserin (10 μM) (final concentration of DMSO=0.2%). After 1 h, the media is removed and replaced with new Neurobasal media containing 1× B27 supplement, 1% penicillin-streptomycin, 0.5 mM glutamine, and 12.5 μM glutamate. The cells are allowed to grow for an additional 71 h before being fixed, stained, and imaged.

Neurite Outgrowth Assay. Rat cortical neurons (20,000 cells/well) are freshly isolated from embryonic day 18 rats and cultured in Neurobasal Medium (+B27). The cultured cells are plated in 96 well-plates (avoiding external wells). At DIV 4, the neurons are treated with compound or control (10 μM) for 1 hour followed by complete washout of the compound. At DIV 7, the neurons are analyzed. The experiments are performed in triplicate. Neurite outgrowth are measured analyzing the following parameters: Number of Cell Bodies, total neurite length (pixels), Root Count, Segments, Extremities Count and node points. Changes in the pattern of neurite outgrowth of the neurons are analyzed by immunocytochemistry against b-III-tubulin. Images are acquired by the Cellinsight CX7 from Thermo Fisher and analyzed using its software. Results are generated in the equipment for maximum neurite length, extremity count, root count, dendrite branch points, and total neurite length. The results are compared to DMSO control, representing the fold-change in neuronal outgrowth. Examples of the presently disclosed compounds increase neuronal outgrowth, supporting their use in increasing plasticity and in the treatment of brain disease.

Serotonin and Opioid Receptor Functional Assays. Functional assay screens at 5-HT and opioid receptors are performed in parallel using the same compound dilutions and 384-well format high-throughput assay platforms. Assays assess activity at all human isoforms of the receptors, except where noted for the mouse 5-HT_{2A} receptor. Receptor constructs in pcDNA vectors are generated from the Presto-Tango GPCR library with minor modifications. All compounds are serially diluted in drug buffer (HBSS, 20 mM HEPES, pH 7.4 supplemented with 0.1% bovine serum albumin and 0.01% ascorbic acid) and dispensed into 384-well assay plates using a FLIPR^{TETRA} (Molecular Devices). Every plate included a positive control such as 5-HT (for all 5-HT receptors), DADLE (DOR), salvinorin A (KOR), and DAMGO (MOR). For measurements of 5-HT2A, 5-HT2B, and 5-HT2C Gq-mediated calcium flux function, HEK Flp-In 293 T-Rex stable cell lines (Invitrogen) are loaded with Fluo-4 dye for one hour, stimulated with compounds and read for baseline (0-10 seconds) and peak fold-over-basal fluorescence (5 minutes) at 25° C. on the FLIPR^{TETRA}. For measurement of 5-HT6 and 5-HT7a functional assays, Gs-mediated cAMP accumulation is detected using the split-luciferase GloSensor assay in HEKT cells measuring luminescence on a Microbeta Trilux (Perkin Elmer) with a 15 min drug incubation at 25° C. For 5-HT1A, 5-HT1B, 5-HT1F, MOR, KOR, and DOR functional assays, Gi/o-mediated cAMP inhibition is measured using the split-luciferase GloSensor assay in HEKT cells, conducted similarly as above, but in combination with either 0.3 μM isoproterenol (5-HT1A, 5-HT1B, 5-HT1F) or 1 μM forskolin (MOR, KOR, and DOR) to stimulate endogenous cAMP accumulation. For measurement of 5-HT1D, 5-HT1E, 5-HT4, and 5-HT5A functional assays, P-arrestin2 recruitment is measured by the Tango assay utilizing HTLA cells expressing TEV fused-P-arrestin2, as described previously with minor modifications. Data for all assays are plotted and non-linear regression is performed using “log(agonist) vs. response” in Graphpad Prism to yield Emax and EC₅₀ parameter estimates.

5HT_{2A} Sensor Assays. HEK293T (ATCC) 5HT_{2A} sensor stable line (sLight1.3s) is generated via lentiviral transduction of HIV-EF1α-sLight1.3 and propagated from a single colony. Lentivirus is produced using 2^{nd} generation lentiviral plasmids pHIV-EF1α-sLight1.3, pHCMV-G, and pCMV-deltaR8.2.

For the screening of the compounds, sLight1.3s cells are plated in 96-well plates at a density of 40000 24-hours prior to imaging. On the day of imaging, compounds solubilized in DMSO are diluted from the 100 mM stock solution to working concentrations of 1 mM, 100 mM and 1 μM with a DMSO concentration of 1%. Immediately prior to imaging, cells growing in DMEM (Gibco) are washed 2× with HBSS (Gibco) and in agonist mode 180 μL of HBSS or in antagonist mode 160 μL of HBSS is added to each well after the final wash. For agonist mode, images are taken before and after the addition of the 20 μL compound working solution into the wells containing 180 μL HBSS. This produces final compound concentrations of 100 mM, 10 mM and 100 nM with a DMSO concentration of 0.1%. For antagonist mode, images are taken before and after addition of 20 μL of 900 nM 5-HT and again after 20 μL of the compound working solutions to produce final concentrations of 100 nM for 5HT and 100 mM, 10 mM and 100 nM for the compounds with a DMSO concentration of 0.1%. Each compound is tested in triplicate (3 wells) for each concentration (100 mM, 10 mM and 100 nM). Additionally, within each plate, 100 nM 5HT and 0.1% DMSO controls are also imaged.

Imaging is performed using the Leica DMi8 inverted microscope with a 40× objective using the FITC preset with an excitation of 460 nm and emission of 512-542 nm. For each well, the cellular membrane where the 5HT_{2A} sensor is targeted is autofocused using the adaptive focus controls and 5 images from different regions within the well are taken with each image processed from a 2×2 binning.

For data processing, the membranes from each image are segmented and analyzed using a custom algorithm written in MATFAB producing a single raw fluorescence intensity value. For each well the 5 raw fluorescence intensity values generated from the 5 images are averaged and the change in fluorescence intensity (dFF) is calculated as:

$$dFF = ( {F}_{sat} - {F}_{apo} ) / {F}_{apo}$$

For both agonist and antagonist modes, the fluorescence intensity values before compound addition in FIBSS only are used as the Fₐₚₒ values while the fluorescence intensity values after compound addition are used as the Fₛₐₜ values.

For agonist mode, data are as percent activation relative to 5HT, where 0 is the average of the DMSO wells and 100 is the average of the 100 μM 5HT wells. For antagonist mode, the inactivation score is calculated as:

$$Inactivation score = ( dFFF ( Compound + 5 HT ) - dFF ( 5 H T ) ) / dFF ( 5 H T )$$

### Example 7: Head-Twitch Response (HTR)

Head-Twitch Response (HTR). The head-twitch response assay is performed as is known to those of skill in the art using both male and female C57BL/6J mice (3 per treatment). The mice are obtained and are approximately 8 weeks old at the time of the experiments. Compounds are administered via intraperitoneal injection (5 mL/kg) using 0.9% saline as the vehicle. As a positive control, 5-MeO-DMT fumarate (2:1 amine/acid) was utilized. Behavior was videotaped, later scored by two blinded observers, and the results were averaged (Pearson correlation coefficient=0.93).

### Background: Hallucinogen-Induced Head Shakes and Twitches

Mice administered LSD were reported by Keller and Umbreit (1956) to respond with rapid and violent head shaking that does not occur in normal mice. This response was found to be remarkably consistent when scored by different observers across laboratories. The head-shake response is elicited by a wide variety of known hallucinogens such as LSD, psilocybin, psilocin, N,N-dimethyltryptamine (DMT), and mescaline as well as serotonin-releasing agents and direct 5-HT2 agonists (Canal and Morgan 2012). 2,5-dimethoxy-4-iodoamphetamine (DOI) has also been reported to elicit head-shakes in rats (Arnt and Hyttel 1989, Kennett et al., 1994) and head-twitches in mice Darmani et al., 1990), both of which were blocked by administration of the fairly selective 5-HT2A antagonist ketanserin. Later studies have confirmed 5-HT2A receptors are the primary, direct mediators of the response and that the headshake response in rats is essentially the same as head-twitches in mice, at least in regards to similarity in appearance and 5-HT2A receptor dependence (Canal and Morgan 2012). The head twitch and head shake response in mice and rats have therefore been widely used to explore the effect of treatments on 5-HT2A receptors in vivo.

### REFERENCES

Arnt J, Hyttel J. (1989). Facilitation of 8-OHDPAT-induced forepaw treading of rats by the 5-HT2 agonist DOI. Eur. J. Pharmacol., 161:45.
Canal C E., Morgan D. (2012). Head-twitch response in rodents induced by the hallucinogen 2,5-dimethoxy-4-iodoamphetamine: a comprehensive history, a re-evaluation of mechanisms and its utility as a model. Drug Test Anal. 4, 556-576.
Darmani N A, Martin B R, Pandey U, Glennon R A. (1990). Do functional relationships exist between 5-HT1A and 5-HT2 receptors?Pharmacol Biochem Behav., 36: 901-6.
Keller D L, Umbreit W W. (1956). Permanent alteration of behavior in mice by chemical and psychological means. Science, 124: 723.
Kennett G A, Wood M D, Glen A, Grewal S, Forbes I, Gadre A, Blackburn T P. (1994). In vivo properties of SB 200646A, a 5-HT2C/2B receptor antagonist. Br J Pharmacol. 111: 797-802.

Protocol 1: Effect of AAZ, five representative compounds of the application, and 5-MeO-DMT on head twitches in mice.

### Animals

24 Male C57BL/6J mice (approximately 25 g) were group housed in a stock room. Animals were maintained under a 12 h light/dark cycle, at 23° C. with humidity controlled according to Home Office regulations.

### Formulation

Compounds 1, 2, 4, 151, 93, AAZ literature compound and 5-MeO-DMT (all compounds supplied as free bases) were each formulated in DMSO:saline [10:90] at a concentration of 2 mg/mL to give a dose of 10 mg/kg when administered ip in a 5 mL/kg dosing volume.

### Procedure

At T=−60 min, C57BL/6J mice were individually housed into transparent observation cages with bedding removed and left to habituate.

At T=0 h, groups of 3 mice were dosed intraperitoneally with either Vehicle, Compound 1, Compound 2, Compound 4, Compound 151, Compound 93, AAZ literature compound, or 5-MeO-DMT each at 10 mg/kg. Following dosing, mice were replaced into the observation cages and head twitch behavior was monitored for 40 min after agonist dosing.

TABLE 5
Synopsis of mouse twitch test schedule

| | | | T = 0-40 min |
|---|---|---|---|
| | 60 min pre-test | T = 0 pre test | Assess no of head |
| Grp | Place in test cage | Treatment IP | shakes 40 min per |
| (n) | to habituate | (5 mL/kg DMSO: saline) | mouse |
| 3 | Yes | Vehicle | Yes |
| 3 | Yes | Compound 1 ((R)-1-(5-cyclopropoxy-1H- | Yes |
| | | indol-1-yl)-N,N-dimethylpropan-2-amine) | |
| | | 10 mg/kg | |
| 3 | Yes | Compound 2 ((2R)-N,N-dimethyl-1-[5- | Yes |
| | | (pentafluoro-λ⁶-sulfanyl)indol-1- | |
| | | yl]propan-2-amine) 10 mg/kg | |
| 3 | Yes | Compound 4 ((2R)-1-(5- | Yes |
| | | cyclopropylsulfonylindol-1-yl)-N,N- | |
| | | dimethyl-propan-2-amine) 10 mg/kg | |
| 3 | Yes | Compound 151 ((2R)-1,1,1,2,3,3- | Yes |
| | | hexadeuterio-N,N-dimethyl-3-[5- | |
| | | (trideuteriomethoxy)indol-1-yl]propan-2- | |
| | | amine) 10 mg/kg | |
| 3 | Yes | Compound 93 ((R/S)-1-(6- | Yes |
| | | fluoroimidazo[1,5-a]pyridin-1-yl)-N,N- | |
| | | dimethylpropan-2-amine)* | |
| 3 | Yes | AAZ reference compound ((R)-1-(5- | Yes |
| | | methoxy-1H-indol-1-yl)-N,N- | |
| | | dimethylpropan-2-amine) 10 mg/kg | |
| 3 | Yes | 5-MeO-DMT 10 mg/kg | Yes |

*Study of compound 93 was conducted on a separate day.

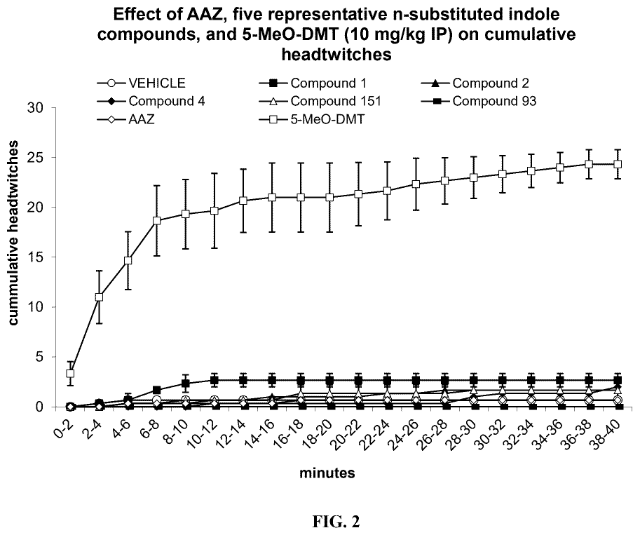

FIG. 2 provides a graph showing average cumulative head twitches induced by AAZ, five representative compounds of the application, and 5-MeO-DMT.

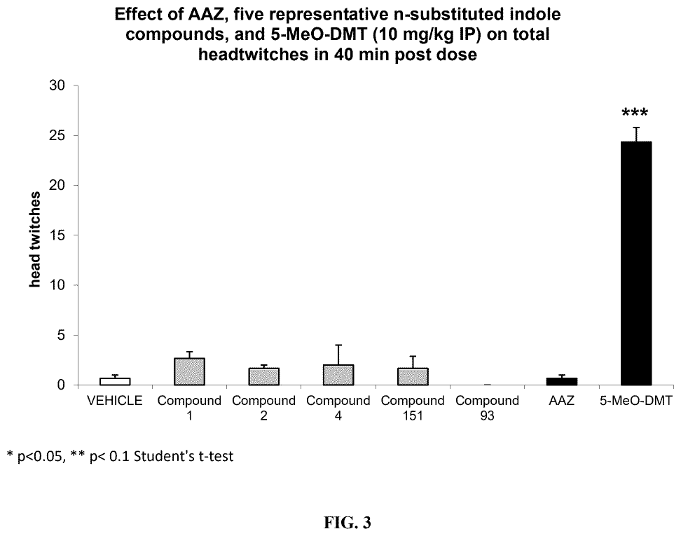

FIG. 3 provides a bar chart showing total average head twitches induced by AAZ, five representative compounds of the application, and 5-MeO-DMT in the 40 minutes post-dose.

As illustrated in FIGS. 2 and 3, the five representative compounds of the application did not produce a significant head-twitch response compared to placebo. Only 5-MeO-DMT produced significant increases in head-twitch. This experiment shows that these compounds are not expected to produce hallucinations in humans. Hallucinations are a treatment limiting side effect and the lack of hallucinatory activity shows that these compounds have advantages over psychedelics such as ibogaine that cause treatment limiting hallucinations.

Forced Swim Test (FST). Male C57/BL6J mice (9-10 weeks old at time of experiment) are obtained. After 1 week in the vivarium each mouse is handled for approximately 1 minute by the experimenter for 3 consecutive days leading up to the first FST. All experiments are carried out by the same experimenter who performs handling. During the FST, mice undergo a 6 min swim session in a clear Plexiglas cylinder 40 cm tall, 20 cm in diameter, and filled with 30 cm of 24±1° C. water. Fresh water is used for every mouse. After handling and habituation to the experimenter, drug-naive mice first undergo a pretest swim to more reliably induce a depressive phenotype in the subsequent FST sessions. Immobility scores for all mice are determined after the pre-test and mice are randomly assigned to treatment groups to generate groups with similar average immobility scores to be used for the following two FST sessions. The next day, the animals receive intraperitoneal injections of experimental compounds (20 mg/kg), a positive control (ketamine, 3 mg/kg), or vehicle (saline). The animals are subjected to the FST 30 mins after injection and then returned to their home cages. All FSTs are performed between the hours of 8 am and 1 μm. Experiments are video-recorded and manually scored offline. Immobility time—defined as passive floating or remaining motionless with no activity other than that needed to keep the mouse's head above water—is scored for the last 4 min of the 6 min trial.

Alcohol Use Disorder Model: To assess the anti-addictive potential of the present compounds, an alcohol drinking paradigm that models heavy alcohol use and binge drinking behavior in humans is employed. Using a 2-bottle choice setup (20% ethanol (v/v), EtOH vs. water, H₂O), mice are subjected to repeated cycles of binge drinking and withdrawal over the course of 7 weeks.

This schedule results in heavy EtOH consumption, binge drinking-like behavior, and generates blood alcohol content equivalent to that of human subjects suffering from alcohol use disorder (AUD). Next, compounds of the invention are administered via intraperitoneal injection 3 h prior to a drinking session, and EtOH and H₂O consumption is monitored. Effective compounds of the invention robustly reduce binge drinking during the first 4 h, decreasing EtOH consumption. With exemplary compounds, consumption of ethanol is lower for at least two days following administration with no effect on water intake. Efficacy in this assay suggests the present compounds are useful for the treatment of AUD.

Statistical analysis. Treatments are randomized, and data are analyzed by experimenters blinded to treatment conditions. Statistical analyses are performed using GraphPad Prism (version 8.1.2). The specific tests are, F-statistics and degrees of freedom. All comparisons are planned prior to performing each experiment. For dendritogenesis experiments a one way ANOVA with Dunnett's post hoc test is deemed most appropriate. Ketamine is included as a positive control to ensure that the assay is working properly.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1.-46. (canceled)

47. A compound of Formula IV: 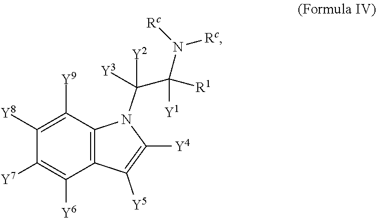
or a pharmaceutically acceptable salt or stereoisomer thereof, wherein:
R¹ is CH₃ or CD₃
Y¹ is H, D, CH₃, or CD₃;
Y² is H or D;
Y³ is H or D;
Y⁴ is H or D;
Y⁵ is H or D;
Y⁶ is H or D;
Y⁷ is H or D;
Y⁸ is H or D;
Y⁹ is H or D; and
each R^{c} is, independently, CH₃ or CD₃;
wherein at least one of Y¹, Y², and Y³ is D, and/or R¹ is CD³.

48. The compound of claim 47, or a pharmaceutically acceptable salt or stereoisomer thereof, wherein Y¹ is D.

49. The compound of claim 47, or a pharmaceutically acceptable salt or stereoisomer thereof, wherein Y² is D.

50. The compound of claim 47, or a pharmaceutically acceptable salt or stereoisomer thereof, wherein Y³ is D.

51. The compound of claim 47, or a pharmaceutically acceptable salt or stereoisomer thereof, wherein R¹ is CD₃.

52. The compound of claim 47, or a pharmaceutically acceptable salt or stereoisomer thereof, wherein;
Y¹ is D;
Y² is D;
Y³ is D; and
R¹ is CD₃.

53. The compound of claim 47, or a pharmaceutically acceptable salt or stereoisomer thereof, wherein:
Y⁴ is H;
Y⁵ is H;
Y⁶ is H;
Y⁷ is H;
Y⁸ is H; and
Y⁹ is H.

54. The compound of claim 52, or a pharmaceutically acceptable salt or stereoisomer thereof, wherein
Y⁴ is H;
Y⁵ is H;
Y⁶ is H;
Y⁷ is H;
Y⁸ is H; and
Y⁹ is H.

55. The compound of claim 47, or a pharmaceutically acceptable salt or stereoisomer thereof, wherein each R^{c} is CD₃.

56. The compound of claim 52, or a pharmaceutically acceptable salt or stereoisomer thereof, wherein each R^{c} is CD₃.

57. The compound of claim 54, or a pharmaceutically acceptable salt or stereoisomer thereof, wherein each R^{c} is CD₃.

58. The compound of claim 47, wherein the compound is selected from 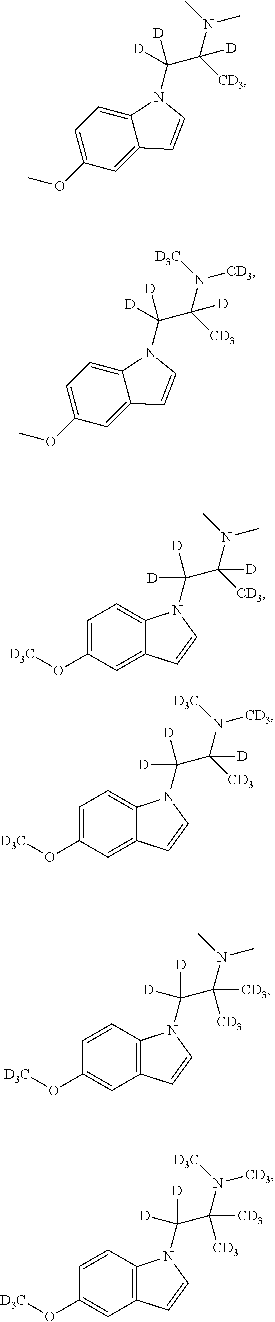 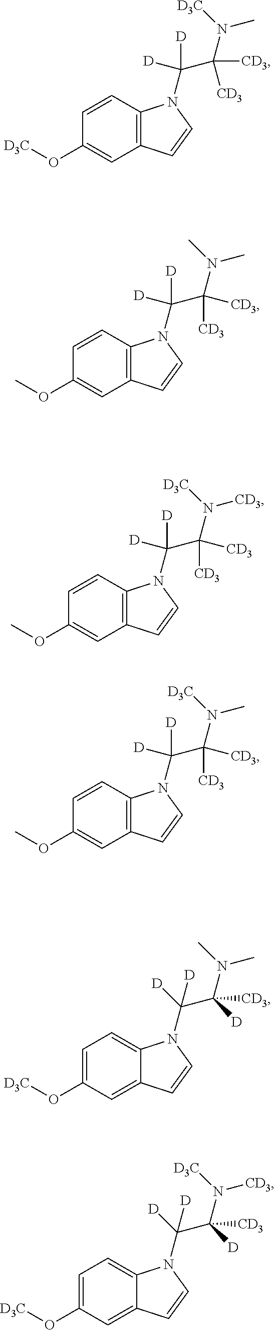 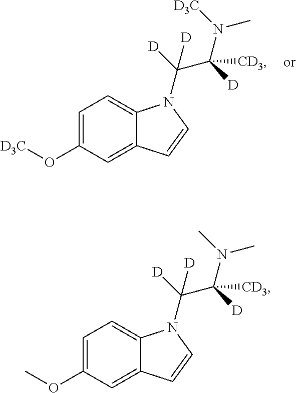
or a pharmaceutically acceptable salt thereof.

59. The compound of claim 47, wherein the compound is selected from 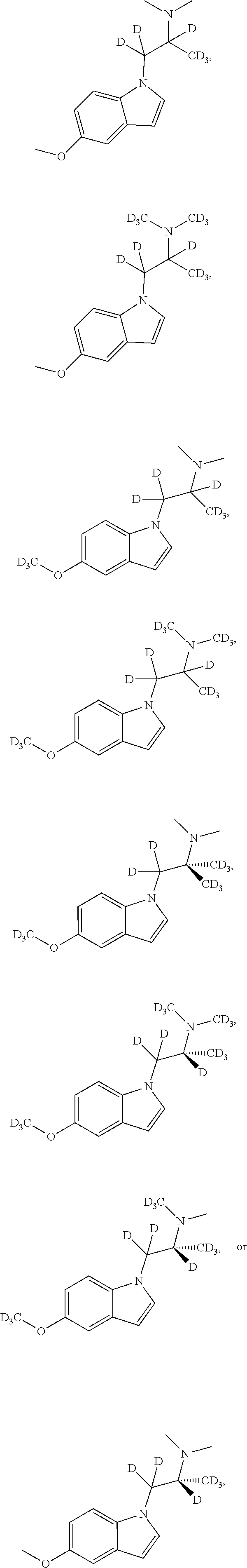
or a pharmaceutically acceptable salt thereof.

60. The compound of claim 47, wherein the compound is selected from 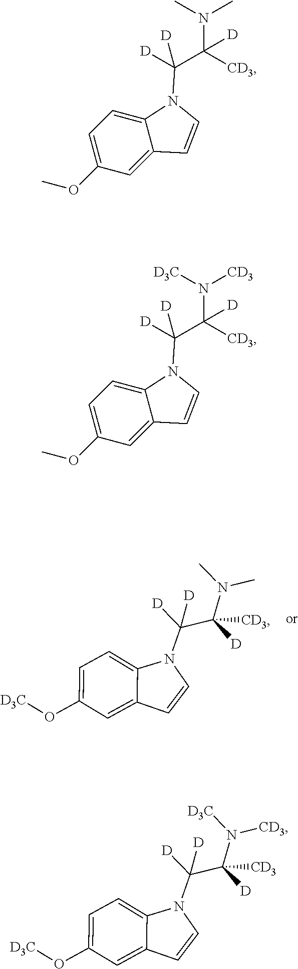 [SMILES: [2H]C([2H])([2H])C([2H])(N(C)C)C([2H])([2H])n1ccc2cc(OC)ccc21 ; [2H]C([2H])([2H])N(C([2H])([2H])[2H])C([2H])(C([2H])([2H])[2H])C([2H])([2H])n1ccc2cc(OC)ccc21 ; [2H]C([2H])([2H])Oc1ccc2c(ccn2C([2H])([2H])[C@]([2H])(N(C)C)C([2H])([2H])[2H])c1 ; "or" ; [2H]C([2H])([2H])Oc1ccc2c(ccn2C([2H])([2H])[C@]([2H])(N(C([2H])([2H])[2H])C([2H])([2H])[2H])C([2H])([2H])[2H])c1]
or a pharmaceutically acceptable salt thereof.

61. A compound selected from 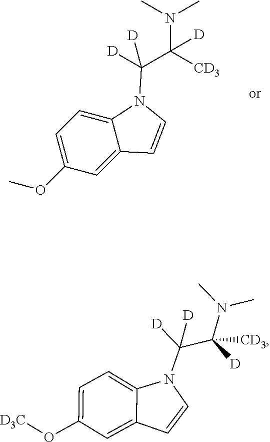 [SMILES: [2H]C([2H])([2H])C([2H])(N(C)C)C([2H])([2H])n1ccc2cc(OC)ccc21 ; [2H]C([2H])([2H])Oc1ccc2c(ccn2C([2H])([2H])[C@]([2H])(N(C)C)C([2H])([2H])[2H])c1]
or a pharmaceutically acceptable salt thereof.

62. A compound selected from 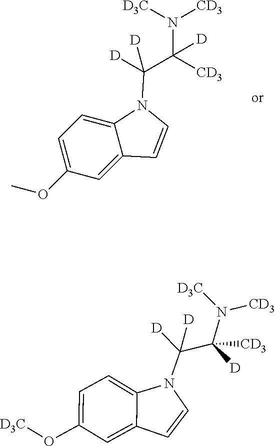 [SMILES: [2H]C([2H])([2H])N(C([2H])([2H])[2H])C([2H])(C([2H])([2H])[2H])C([2H])([2H])n1ccc2cc(OC)ccc21 ; [2H]C([2H])([2H])Oc1ccc2c(ccn2C([2H])([2H])[C@]([2H])(N(C([2H])([2H])[2H])C([2H])([2H])[2H])C([2H])([2H])[2H])c1]
or a pharmaceutically acceptable salt thereof.

63. A pharmaceutical composition comprising a compound, or pharmaceutically acceptable salt thereof, of claim 47.

64. A pharmaceutical composition comprising a compound, or pharmaceutically acceptable salt thereof, of claim 60.

65. A method for treating a neurological disorder or a psychiatric disorder, or both, comprising administering a compound of claim 47, or a pharmaceutically acceptable salt thereof, to a subject in need thereof.

66. The method of claim 65, wherein the neurological disorder or psychiatric disorder, or both, comprises treatment resistant depression, suicidal ideation, major depressive disorder, anxiety, bipolar disorder, schizophrenia, or substance use disorder.
